(19) United States
(12) Patent Application Publication
(10) Pub. No.: US 2025/0179083 A1
(43) Pub. Date: Jun. 5, 2025

(54) TRICYCLIC TRIAZOLO COMPOUNDS AS DGK INHIBITORS

(71) Applicant: Incyte Corporation, Wilmington, DE (US)

(72) Inventors: Joshua Hummel, Hockessin, DE (US); Liana Hie, Wilmington, DE (US); Jacob J. Lacharity, Chesterbrook, PA (US); Xiaolei Li, New Castle, DE (US); Ding-Quan Qian, Newark, DE (US); Xiaozhao Wang, Moorestown, NJ (US); Bo Wei, Claymont, DE (US)

(21) Appl. No.: 18/968,107

(22) Filed: Dec. 4, 2024

Related U.S. Application Data
(60) Provisional application No. 63/607,001, filed on Dec. 6, 2023.
(60) Provisional application No. 63/606,299, filed on Dec. 5, 2023.

(51) Int. Cl. C07D 487/14; A61K 31/496; A61K 31/519; C07D 471/14

(52) U.S. Cl. CPC C07D 487/14; A61K 31/496; A61K 31/519; C07D 471/14

(57) ABSTRACT

The present application provides tricyclic triazolo compounds that modulate the activity of diacylglycerol kinase (DGK), which are useful in the treatment of various diseases, including cancer.

### TECHNICAL FIELD

The present invention provides tricyclic triazolo compounds that modulate the activity of diacylglycerol kinase (DGK) and are useful in the treatment of diseases related to diacylglycerol kinase, including cancer.

### BACKGROUND

Diacylglycerol kinases (DGKS) are a family of enzymes that regulate many biological processes, including cellular proliferation, migration, immunity and pathogenesis of diseases such as cancer. In mammalian systems, there are ten DGK family members classified into five subtypes based on shared common domains (Sakane F. et al., Int. J. Mol. Sci., 2020. 21: p 6794-6829). The diverse and specific cellular function of individual DGK isoforms is regulated through their tissue restricted expression, localization within cells and interactions with regulatory proteins (Joshi, R. P. and Koretzky, G. A., Int. J. Mol. Sci., 2013. 14: p 6649-6673).

In T lymphocytes, DGKα and ζ are the dominant DGK isoforms expressed (Krishna, S. and Zhong, X.-P., Front Immunol., 2013. 4:178). Specifically, in response to T cell receptor (TCR) activation, phospholipase Cγ1 (PLC□1) hydrolyzes membrane phospholipid PIP2 to produce diacylglycerol (DAG) (Krishna, S. and Zhong, X.-P., Front Immunol., 2013. 4:178; Riese, M. J. et al., Front Cell Dev Biol., 2016. 4:108). In turn, DAG functions as a second messenger to recruit RasGRP1 and PKCθ to the cell membrane and thereby initiates multiple downstream signaling events resulting in T cell activation. To prevent hyperactivation of T cells, DGKα and ζ tightly regulate the levels of intracellular DAG by phosphorylating DAG to produce phosphatidic acid (PA). Both mouse and human cell line genetic studies support the important regulatory role of DGKα and ζ in T cell activation. Knockout or depletion of DGKα and ζ has been reported to enhance T cell activation, cytokine production and proliferation. Furthermore, knockout of both DGKα and ζ show even greater T-cell activation over individual knockouts, indicating a non-redundant role of these two isoforms (Riese, M. J. et al., Cancer Res., 2013. 73:p 3566-3577; Jung, I.-Y. et al., Cancer Res., 2018. 78: p 4692-4703). Thus, DGKα and ζ, by regulating cellular DAG levels link lipid metabolism and intracellular signaling cascades and function as key regulators of T cell activation.

Cytotoxic T lymphocytes (CTLs) are a major component of the adaptive immune system that recognize and kill cells with bacterial or viral infections, or cells displaying abnormal proteins, such as tumor antigens. However, cancer cells can evolve to utilize multiple mechanisms that mimic peripheral immune tolerance to avoid immune surveillance and killing by CTLs. Such mechanisms include downregulation of antigen presentation, suppression of T cell function through increased expression of inhibitory molecules, as well as increased production of immunosuppressive proteins in the tumor microenvironment (Speiser, D. E. et al., Nat. Rev. Immunol., 2016. 16: p. 599-611, Gonzalez H. et al., Genes & Dev., 2018. 32:p 1267-1284). Immune checkpoint therapy (ICT) by blocking inhibitory molecules such as PD(L)-1 and CTLA4, can restore T cell activity and have been clinically useful in treating many different types of cancers. However, only subsets of patients respond to ICT due to primary or acquired resistance (Sharma, P. et al., Cell. 2017. 168: p 707-723). Thus, despite the significant recent clinical successes of immunotherapies to treat cancer, resistance remains a challenge (Sharma, P., et al., Cancer Discov., 2021. 11: p 838-857).

Overexpression of DGKα and ζ has been observed in tumor infiltrating lymphocytes (TILs) from human tumors and proposed to suppress T cell function. Importantly, significant immune-mediated antitumor activity has been shown in DGKα and DGKζ deficient mouse models (Merida, I. et al., Adv. Biol. Regul., 2017. 63:p 22-31, Prinz, P. U. et al., J. Immunol., 2012. 188:p 5990-6000). Furthermore, DGKα and DGKζ deficient T cells are resistant to several immunosuppressive factors within the tumor microenvironment such as TGFβ, PGE2 and adenosine, and to other T cell inhibitory pathways such as PD(L)-1 mediated immune suppression (Riese, M. J. et al., Cancer Res., 2013. 73:p 3566-77; Jung, I.-Y. et al. (2018) Cancer Res., 2018. 78:p 4692-4703; Arranz-Nicolas, J. et al., Cancer Immunol. Immunother., 2018. 67:p 965-980; Riese, M. J. et al., Front. Cell Dev. Biol., 2016. 4:108). Thus DGKα and DGKζ are attractive targets as immunotherapies alone or in combination with current ICT therapies such as PD(L)-1 and CTLA4. By targeting T cell lipid metabolism, DGKα and DGKζ inhibition can potentially restore antitumor immunity in subsets of patient who have primary or acquired immune resistance and are consequently refractory to current ICTs. In addition to its function in T lymphocytes, DGKα and DGKζ, by regulating DAG level in cancer cells, have also been reported to directly contribute to cancer proliferation, migration, invasion and survival. Thus, DGK inhibition may have direct antitumor effect by interfering with tumor intrinsic oncogenic survival pathways (Cooke, M. and Kaznietz, M. G., Sci. Signal., 2022. 15:eabo0264).

Compounds in this application may have selective activities towards one or both DGKα and DGKζ. These DGK inhibitors alone or in combination with other therapeutic agent(s) can be used in treatment of cancer.

### SUMMARY

The present invention relates to, inter alia, compounds of Formula I:

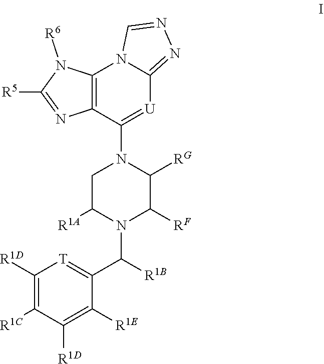

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides methods of inhibiting an activity of diacylglycerol kinase (DGK), comprising contacting the kinase with a compound of Formula I, or a pharmaceutically acceptable salt thereof.

The present invention further provides methods of treating a disease or a disorder associated with expression or activity of a diacylglycerol kinase (DGK) in a patient by administering to a patient a therapeutically effective amount of a compound of Formula I, or a pharmaceutically acceptable salt thereof.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

The present application provides a compound of Formula I.

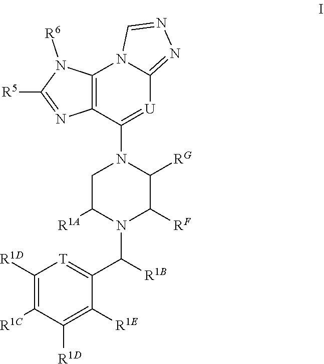

or a pharmaceutically acceptable salt thereof, wherein:
U is CH or N;
T is CH, C—R^{1E}, or N;
R^{1A} is H or C₁₋₃ alkyl;
R^{1B} is selected from H, C₁₋₆ alkyl, C₁₋₆ haloalkyl, phenyl, pyridinyl, and C₃₋₇ cycloalkyl, wherein the phenyl, pyridinyl, and C₃₋₇ cycloalkyl are each optionally substituted with 1, 2, or 3 substituents independently selected from halo and C₁₋₃ haloalkyl;
R^{1C} is selected from halo, C₁₋₃ haloalkyl, and C₁₋₃ alkoxy;
each R^{1D} is independently H, C₁₋₃ haloalkyl, or halo;
each R^{1E} is independently H or halo;
or, R^{1C} and R^{1D}, together with the atoms of the phenyl or piperazyl ring form a 10 membered bicyclic heteroaryl, which is optionally substituted with C₁₋₃ haloalkyl;
R^{1F} is H;
or, R^{1A} and R^{1F}, together with the atoms of the piperazyl ring, form an 8 membered bridged heterocycloalkyl; and
R^{1G} is H or C₁₋₃ alkyl;
R⁵ is H or C₁₋₃ alkyl; and
R⁶ is selected from C₃₋₇ cycloalkyl-C₁₋₃ alkyl- and (4-7 membered heterocycloalkyl)-C₁₋₃ alkyl-, wherein the C₃₋₇ cycloalkyl-C₁₋₃ alkyl- and (4-7 membered heterocycloalkyl)-C₁₋₃ alkyl- are each optionally substituted with 1 or 2 substituents independently selected from halo, C₁₋₃ alkyl, C₁₋₃ haloalkyl, CN, and OH.

In some embodiments, U is CH.

In some embodiments, U is N.

In some embodiments, T is CH.

In some embodiments, T is N.

In some embodiments, T is C—R^{1E}.

In some embodiments, U is CH and T is CH.

In some embodiments, U is N and T is CH.

In some embodiments, U is N and T is N.

In some embodiments, R^{1A} is C₁₋₃ alkyl.

In some embodiments, R^{1A} is methyl.

In some embodiments, R^{1A} is ethyl.

In some embodiments, R^{1B} is H.

In some embodiments, R^{1B} is C₁₋₆ alkyl, which is optionally substituted with 1, 2, or 3 substituents independently selected from halo and C₁₋₃ haloalkyl.

In some embodiments, R^{1B} is C₁₋₆ alkyl.

In some embodiments, R^{1B} is selected from methyl, isopropyl, and isobutyl.

In some embodiments, R^{1B} is C₁₋₆ haloalkyl.

In some embodiments, R^{1B} is triflouroisobutyl.

In some embodiments, R^{1B} is 3,3,3-triflouroisobutyl.

In some embodiments, R^{1B} is phenyl, wherein the phenyl is optionally substituted with 1, 2, or 3 substituents independently selected from halo and C₁₋₃ haloalkyl.

In some embodiments, R^{1B} is phenyl, wherein the phenyl is optionally substituted with 1, 2, or 3 substituents independently selected from halo.

In some embodiments, R^{1B} is phenyl, wherein the phenyl is optionally substituted with 1, 2, or 3 substituents independently selected from chloro and fluoro.

In some embodiments, R^{1B} is fluorophenyl or chlorophenyl.

In some embodiments, R^{1B} is fluorophenyl.

In some embodiments, R^{1B} is chlorophenyl.

In some embodiments, R^{1B} is 4-fluorophenyl or 4-chlorophenyl.

In some embodiments, R^{1B} is 4-fluorophenyl. In some embodiments, R^{1B} is 4 chlorophenyl.

In some embodiments, R^{1B} is pyridinyl, wherein the pyridinyl is optionally substituted with 1, 2, or 3 substituents independently selected from halo and C₁₋₃ haloalkyl.

In some embodiments, R^{1B} is pyridinyl, wherein the pyridinyl is optionally substituted with 1, 2, or 3 substituents independently selected from chloro and trifluoromethyl.

In some embodiments, R^{1B} is chloropyridinyl or trifluoromethylpyridinyl.

In some embodiments, R^{1B} is chloropyridinyl.

In some embodiments, R^{1B} is trifluoromethylpyridinyl.

In some embodiments, R^{1B} is 3-chloropyridinyl or 3-trifluoromethylpyridinyl.

In some embodiments, R^{1B} is 3-chloropyridinyl.

In some embodiments, R^{1B} is 3-trifluoromethylpyridinyl.

In some embodiments, R^{1B} is 3-chloropyridin-6-yl or 3-trifluoromethylpyridin-6-yl.

In some embodiments, R^{1B} is 3-chloropyridin-6-yl.

In some embodiments, R^{1B} is 3-trifluoromethylpyridin-6-yl.

In some embodiments, R^{1B} is C₃₋₇ cycloalkyl, wherein the C₃₋₇ cycloalkyl is optionally substituted with 1, 2, or 3 independently selected halo substituents.

In some embodiments, R^{1B} is cyclopropyl or cyclobutyl, wherein the cyclopropyl and cyclobutyl are each optionally substituted with 1, 2, or 3 independently selected halo substituents.

In some embodiments, R^{1B} is selected from cyclopropyl, difluorocyclopropyl, and difluorocyclobutyl.

In some embodiments, R^{1B} is cyclopropyl.

In some embodiments, R^{1B} is difluorocyclopropyl.

In some embodiments, R^{1B} is difluorocyclobutyl.

In some embodiments, R^{1B} is selected from cyclopropyl, 1,1-difluorocycloprop-2-yl, and 1,1-difluorocyclobut-3-yl.

In some embodiments, R^{1B} is 1,1-difluorocycloprop-2-yl.

In some embodiments, R^{1B} is selected from 1,1-difluorocyclobut-3-yl.

In some embodiments, R^{1B} is selected from H, methyl, isopropyl, isobutyl, triflouroisobutyl, fluorophenyl, chlorophenyl, chloropyridinyl, trifluoromethylpyridinyl, cyclopropyl, difluorocyclopropyl, and difluorocyclobutyl.

In some embodiments, R^{1B} is selected from H, methyl, isopropyl, isobutyl, 3,3,3-triflouroisobutyl, 4-fluorophenyl, 4-chlorophenyl, 3-chloropyridin-6-yl, 3-trifluoromethylpyridin-6-yl, cyclopropyl, 1,1-difluorocycloprop-2-yl, and 1,1-difluorocyclobut-3-yl.

In some embodiments, R^{1C} is halo.

In some embodiments, R^{1C} is fluoro, chloro, or bromo.

In some embodiments, R^{1C} is fluoro.

In some embodiments, R^{1C} is chloro.

In some embodiments, R^{1C} is bromo.

In some embodiments, R^{1C} is C₁₋₃ haloalkyl.

In some embodiments, R^{1C} is difluoromethyl or trifluoromethyl.

In some embodiments, R^{1C} is difluoromethyl.

In some embodiments, R^{1C} is trifluoromethyl.

In some embodiments, R^{1C} is C₁₋₃ alkoxy.

In some embodiments, R^{1C} is methoxy.

In some embodiments, each R^{1D} is H.

In some embodiments, each R^{1D} is halo.

In some embodiments, each R^{1D} is C₁₋₃ haloalkyl.

In some embodiments, each R^{1D} is independently H, fluoro, chloro, or trifluoromethyl.

In some embodiments, one R^{1D} is H and a second R^{1D} is selected from fluoro, chloro, and trifluoromethyl.

In some embodiments, R^{1C} and R^{1D}, together with the atoms of the phenyl or piperazyl ring form a 10 membered bicyclic heteroaryl, which is optionally substituted with C₁₋₃ haloalkyl.

In some embodiments, R^{1C} and R^{1D}, together with the atoms of the phenyl or piperazyl ring form a 10 membered bicyclic heteroaryl, which is substituted with C₁₋₃ haloalkyl.

In some embodiments, R^{1C} and R^{1D}, together with the atoms of the phenyl or piperazyl ring form a 10 membered bicyclic heteroaryl, which is substituted with trifluoromethyl.

In some embodiments, each R^{1E} is H.

In some embodiments, each R^{1E} is halo.

In some embodiments, each R^{1E} is independently H or fluoro.

In some embodiments, each R^{1E} is H or fluoro.

In some embodiments, each R^{1E} is fluoro.

In some embodiments, R^{1F} is H.

In some embodiments, R^{1A} and R^{1F}, together with the atoms of the piperazyl ring, form an 8 membered bridged heterocycloalkyl.

In some embodiments, R^{1G} is H or C₁₋₃ alkyl.

In some embodiments, R^{1G} is C₁₋₃ alkyl.

In some embodiments, R^{1G} is methyl.

In some embodiments, R^{1G} is H.

In some embodiments, R^{1A} and R^{1F}, together with the atoms of the piperazyl ring, form an 8 membered bridged heterocycloalkyl; and

R^{1G} is H.

In some embodiments, R⁵ is H.

In some embodiments, R⁵ is C₁₋₃ alkyl.

In some embodiments, R⁵ is H or methyl.

In some embodiments, R⁵ is methyl.

In some embodiments, R⁶ is C₃₋₇ cycloalkyl-C₁₋₃ alkyl-, wherein the C₃₋₇ cycloalkyl-C₁₋₃ alkyl- is optionally substituted with 1 or 2 OH substituents.

In some embodiments, R⁶ is cyclopentylmethyl, wherein the cyclopentylmethyl is optionally substituted with OH.

In some embodiments, R⁶ is cyclopentylmethyl, wherein the cyclopentylmethyl is optionally substituted with OH.

In some embodiments, R⁶ is (hydroxycyclopentyl)methyl.

In some embodiments, R⁶ is (1-hydroxycyclopent-2-yl)methyl.

In some embodiments, R⁶ is (4-7 membered heterocycloalkyl)-C₁₋₃ alkyl-, wherein the (4-7 membered heterocycloalkyl)-C₁₋₃ alkyl- is optionally substituted with 1 or 2 substituents independently selected from halo, C₁₋₃ alkyl, C₁₋₃ haloalkyl, CN, and OH.

In some embodiments, R⁶ is (4-7 membered heterocycloalkyl)-C₁₋₃ alkyl-.

In some embodiments, R⁶ is tetrahydrofuranylmethyl.

In some embodiments, R⁶ is (tetrahydrofuran-2-yl)methyl.

In some embodiments:
U is CH or N;
T is CH, C—R^{1E}, or N;
R^{1A} is C₁₋₃ alkyl;
R^{1B} is selected from H, C₁₋₆ alkyl, C₁₋₆ haloalkyl, phenyl, pyridinyl, and C₃₋₇ cycloalkyl, wherein the phenyl, pyridinyl, and C₃₋₇ cycloalkyl are each optionally substituted with 1, 2, or 3 substituents independently selected from halo and C₁₋₃ haloalkyl;
R^{1C} is selected from halo, C₁₋₃ haloalkyl, and C₁₋₃ alkoxy;
each R^{1D} is independently H, fluoro, chloro, or trifluoromethyl;
or R^{1C} and R^{1D}, together with the atoms of the phenyl or piperazyl ring form a 10 membered bicyclic heteroaryl, which is substituted with C₁₋₃ haloalkyl;
each R^{1E} independently is H or fluoro;
R^{1F} is H; or
R^{1A} and R^{1F}, together with the atoms of the piperazyl ring, form an 8 membered bridged heterocycloalkyl;
R^{1G} is H or methyl;
R⁵ is H or C₁₋₃ alkyl; and
R⁶ is C₃₋₇ cycloalkyl-C₁₋₃ alkyl-, wherein the C₃₋₇ cycloalkyl-C₁₋₃ alkyl- is optionally substituted with 1 or 2 OH substituents; or
R⁶ is (4-7 membered heterocycloalkyl)-C₁₋₃ alkyl-.

In some embodiments:
U is CH and T is CH or C—R^{1E}; or
U is N and T is CH or C—R^{1E}; or
U is N and T is N;
R^{1A} is methyl or ethyl;
R^{1B} is selected from H, C₁₋₆ alkyl, C₁₋₆ haloalkyl, phenyl, pyridinyl, and C₃₋₇ cycloalkyl, wherein the phenyl, pyridinyl, and C₃₋₇ cycloalkyl are each optionally substituted with 1, 2, or 3 substituents independently selected from halo and C₁₋₃ haloalkyl;
R^{1C} is selected from fluoro, chloro, bromo, difluoromethyl, trifluoromethyl, and methoxy;
one R^{1D} is H and a second R^{1D} is selected from fluoro, chloro, and trifluoromethyl;
or R^{1C} and R^{1D}, together with the atoms of the phenyl or piperazyl ring form a 10 membered bicyclic heteroaryl, which is substituted with trifluoromethyl;
each R^{1E} is independently H or fluoro;
R^{1F} is H;
or R^{1A} and R^{1F}, together with the atoms of the piperazyl ring, form an 8 membered bridged heterocycloalkyl;
R^{1G} is H or methyl;
R⁵ is H or methyl; and
R⁶ is (hydroxycyclopentyl)methyl or tetrahydrofuranylmethyl.

In some embodiments, the compound of Formula I is a compound of Formula Ia:

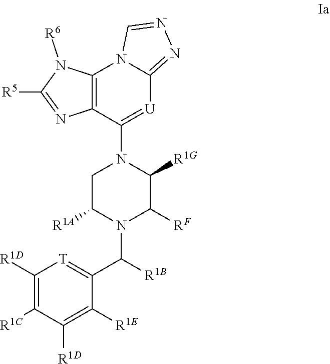

or a pharmaceutically acceptable salt thereof.

In some embodiments, the compound of Formula I is a compound of Formula Ib:

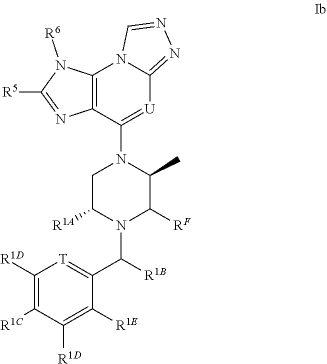

or a pharmaceutically acceptable salt thereof.

In some embodiments, the compound of Formula I is a compound of Formula II:

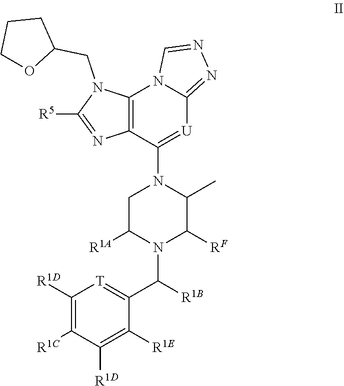

or a pharmaceutically acceptable salt thereof.

In some embodiments, the compound of Formula I is a compound of Formula IIa:

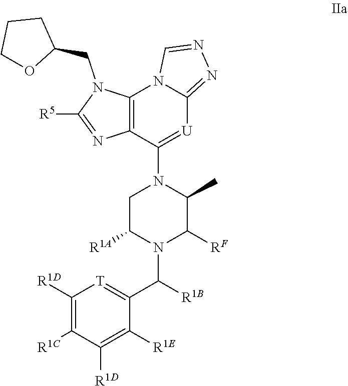

or a pharmaceutically acceptable salt thereof.

In some embodiments, the compound of Formula I is a compound of Formula III:

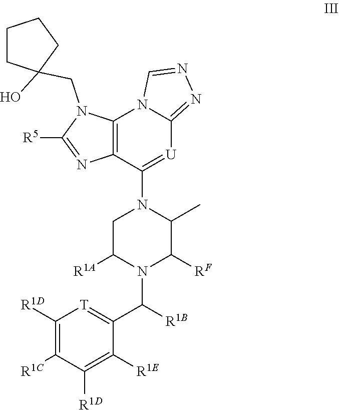

or a pharmaceutically acceptable salt thereof.

In some embodiments, the compound of Formula I is a compound of Formula IIIa:

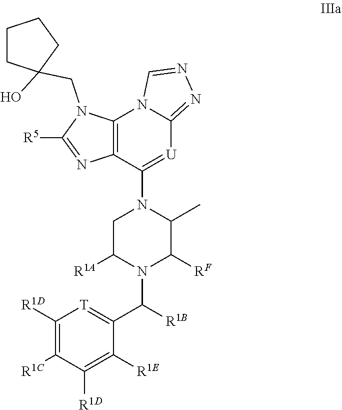

or a pharmaceutically acceptable salt thereof.

In some embodiments, the compound of Formula I is a compound of Formula IV:

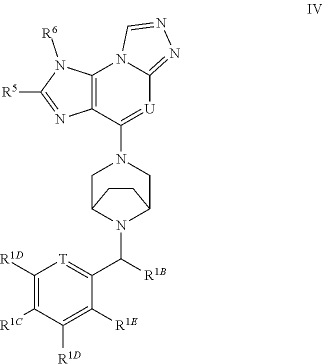

or a pharmaceutically acceptable salt thereof.

In some embodiments, the compound of Formula I is a compound of Formula

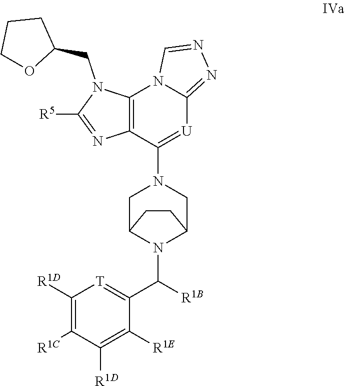

or a pharmaceutically acceptable salt thereof.

In some embodiments, the compound provided herein is selected from:
4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(difluoromethyl)-3-fluorophenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(difluoromethyl)-2-fluorophenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(2-fluoro-4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-2-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(cyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3R)-1-(4-chlorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3S)-1-(4-chlorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(((S)-2,2-difluorocyclopropyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-(3-methyl-1-(4-(trifluoromethyl)phenyl)butyl)piperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3R)-1-(4-chloro-3-fluorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3S)-1-(4-chloro-3-fluorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(5-(trifluoromethyl)pyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-(bis(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((S)-(4-chlorophenyl)(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-(4-chlorophenyl)(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(3-fluoro-4-methoxyphenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-(difluoromethyl)-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-1-(4-bromo-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-1-(4-bromo-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-1-(4-chloro-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-1-(4-chloro-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chloro-2-fluorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-bromophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-5-ethyl-2-methyl-4-(4-(trifluoromethyl)benzyl)piperazin-1-yl)-2-methyl-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-(4-(trifluoromethyl)benzyl)piperazin-1-yl)-2-methyl-1-(((S)-tetrahydrofuran-2-yl)methyl)-1H-[1,2,4]triazolo[3,4-b]purine;
4-((2S,5R)-2,5-dimethyl-4-(4-(trifluoromethyl)benzyl)piperazin-1-yl)-1-(((S)-tetrahydrofuran-2-yl)methyl)-1H-[1,2,4]triazolo[3,4-b]purine;
4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-4-(bis(4-fluorophenyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
1-((4-((2S,5R)-4-(bis(4-chlorophenyl)methyl)-2,5-dimethylpiperazin-1-yl)-1H-[1,2,4]triazolo[3,4-b]purin-1-yl)methyl)cyclopentan-1-ol;
1-((4-((2S,5R)-4-((4-chlorophenyl)(3,3-difluorocyclobutyl)methyl)-2,5-dimethylpiperazin-1-yl)-1H-[1,2,4]triazolo[3,4-b]purin-1-yl)methyl)cyclopentan-1-ol;
1-((4-((2S,5R)-4-((3,3-difluorocyclobutyl)(4-(trifluoromethyl)phenyl)methyl)-2,5-dimethylpiperazin-1-yl)-1H-[1,2,4]triazolo[3,4-b]purin-1-yl)methyl)cyclopentan-1-ol;
4-((2S,5R)-4-(4-chlorobenzyl)-2,5-dimethylpiperazin-1-yl)-1-(((S)-tetrahydrofuran-2-yl)methyl)-1H-[1,2,4]triazolo[3,4-b]purine;
4-((2S,5R)-4-(4-chloro-2-fluorobenzyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-(1-(4-chlorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)(cyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-(4-chlorophenyl)((R)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-(4-chlorophenyl)((R)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-((S)-1-(4-(trifluoromethyl)phenyl)propyl)piperazin-1-yl)-1-(((S)-tetrahydrofuran-2-yl)methyl)-1H-[1,2,4]triazolo[3,4-b]purine;
4-((2S,5R)-2,5-dimethyl-4-((R)-1-(4-(trifluoromethyl)phenyl)propyl)piperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-2,5-dimethyl-4-((S)-2-methyl-1-(6-(trifluoromethyl)quinolin-2-yl)propyl)piperazin-1-yl)-2-methyl-1-(((S)-tetrahydrofuran-2-yl)methyl)-1H-[1,2,4]triazolo[3,4-b]purine;
4-((2S,5R)-2,5-dimethyl-4-((R)-2-methyl-1-(6-(trifluoromethyl)quinolin-2-yl)propyl)piperazin-1-yl)-2-methyl-1-(((S)-tetrahydrofuran-2-yl)methyl)-1H-[1,2,4]triazolo[3,4-b]purine;
4-((2S,5R)-4-((S)-1-(4-chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((R)-1-(4-chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-2,5-dimethyl-4-(1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-2,5-dimethyl-4-(1-(4-(trifluoromethyl)phenyl)ethyl)piperazin-1-yl)-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-(4-chlorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-2-fluorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(3,4-dichlorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-2,6-difluorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-3-fluoro-5-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(3-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-3-fluorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-3-fluorobenzyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-2,5-difluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-2,3-difluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((1R,5S)-8-((3,3-difluorocyclobutyl)(4-(trifluoromethyl)phenyl)methyl)-3,8-diazabicyclo[3.2.1]octan-3-yl)-2-methyl-1-(((S)-tetrahydrofuran-2-yl)methyl)-1H-[1,2,4]triazolo[3,4-b]purine;
4-((1R,5S)-8-(bis(4-fluorophenyl)methyl)-3,8-diazabicyclo[3.2.1]octan-3-yl)-2-methyl-1-(((S)-tetrahydrofuran-2-yl)methyl)-1H-[1,2,4]triazolo[3,4-b]purine;
4-((2S,5R)-2,5-dimethyl-4-((S)-2-methyl-1-(6-(trifluoromethyl)quinolin-2-yl)propyl)piperazin-1-yl)-1-(((S)-tetrahydrofuran-2-yl)methyl)-1H-[1,2,4]triazolo[3,4-b]purine;
4-((2S,5R)-2,5-dimethyl-4-((R)-2-methyl-1-(6-(trifluoromethyl)quinolin-2-yl)propyl)piperazin-1-yl)-1-(((S)-tetrahydrofuran-2-yl)methyl)-1H-[1,2,4]triazolo[3,4-b]purine;
4-((2S,5R)-4-((S)-1-(4-chlorophenyl)propyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chloro-3-fluorophenyl)ethyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-((S)-1-(4-(trifluoromethyl)phenyl)propyl)piperazin-1-yl)-2-methyl-1-(((S)-tetrahydrofuran-2-yl)methyl)-1H-[1,2,4]triazolo[3,4-b]purine; and
4-((2S,5R)-2,5-dimethyl-4-((R)-1-(4-(trifluoromethyl)phenyl)propyl)piperazin-1-yl)-2-methyl-1-(((S)-tetrahydrofuran-2-yl)methyl)-1H-[1,2,4]triazolo[3,4-b]purine;
or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the terms “Cₙ₋ₘ” and “Cₘ₋ₙ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, from 2 to 6 carbon atoms, from 2 to 4 carbon atoms, from 2 to 3 carbon atoms, or 1 to 2 carbon atoms. The term “Cₙ₋ₘ alkyl” is understood to include deuterated analogs of alkyl groups as defined herein, including but not limited to, groups such as trideuteromethyl (CD₃), pentadeuteroethyl (CD₂CD₃), and the like.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. The term “Cₙ₋ₘ alkoxy” is understood to include deuterated analogs of the alkyl moiety of the alkoxy group as defined herein, including but not limited to, groups such as trideuteromethoxy (—OCD₃), pentadeuteroethoxy (—OCD₂CD₃), and the like.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, and the like. In some embodiments, aryl groups have from 5 to 10 carbon atoms. In some embodiments, the aryl group is phenyl or naphthyl. In some embodiments, the aryl is phenyl. The term “aryl” is understood to include deuterated analogs of the aryl groups as defined herein, including but not limited to, groups such as pentadeuterophenyl (i.e., perdeuterophenyl, phenyl-d₅), perdeuteronaphthyl, and the like.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like. The term “Cₙ₋ₘ haloalkyl” is understood to include deuterated analogs of the haloalkyl group as defined herein, including but not limited to, groups such as deuterodifluoromethyl (—CDF₂), dideuterofluoromethyl (—CD₂F), and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl. The term “cycloalkyl” is understood to include deuterated analogs of the cycloalkyl groups as defined herein, including but not limited to, groups such as perdeuterocyclopropyl, perdeuterocyclobutyl, perdeuterocyclopentyl, perdeuterocyclohexyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 15, 3 to 10, 4 to 10, 4 to 15, 5 to 10, 4 to 7, 5 to 7, or 5 to 6 membered heterocycloalkyl groups. Heterocycloalkyl groups can also include spirocycles and bridged rings (e.g., a 5 to 10, or 4 to 15, membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. The term “heterocycloalkyl” is understood to include deuterated analogs of the heterocycloalkyl groups as defined herein, including but not limited to, groups such as perdeuteroazetidinyl, perdeuteropyrrolidinyl, perdeuteropiperidinyl, and the like.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 15 ring-forming atoms, 4 to 10 ring-forming atoms, 4 to 8 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10, or 5-15, membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, tetrahydrothiopheneyl, tetrahydrothiopheneyl 1,1-dioxide, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, 2-azaspiro[3.3]heptanyl, diazaspiro[3.3]heptanyl, azaspiro[3.5]nonanyl, 7-azaspiro[3.5]nonanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl, diazaspiro[5.5]undecanyl, diazaspiro[5.6]dodecanyl, diazaspiro[6.6]tridecanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms. The term “Cₒ₋ₚ cycloalkyl-Cₙ₋ₘ alkyl-” is understood to include deuterated analogs of the cycloalkyl and/or alkyl moieties of the Cₒ₋ₚ cycloalkyl-Cₙ₋ₘ alkyl- groups as defined herein.

As used herein “heterocycloalkyl-Cₙ₋ₘ alkyl-” refers to a group of formula heterocycloalkyl-alkylene-, wherein alkylene linking group has n to m carbon atoms. The term “heterocycloalkyl-Cₙ₋ₘ alkyl-” is understood to include deuterated analogs of the heterocycloalkyl and/or alkyl moieties of the heterocycloalkyl-Cₙ₋ₘ alkyl- groups as defined herein.

As used herein, an “alkyl linking group” or “alkylene linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like. The terms “alkyl linking group” and “alkylene linking group” are understood to include deuterated analogs of the alkylene groups as defined herein.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula I (e.g., compounds of Formula A-1) can be synthesized using the process shown in Scheme 1. As depicted in Scheme 1, a number of methods (e.g., nucleophilic aromatic substitution or a suitable cross-coupling reaction) can be used to access compounds of the general Formula 1-2. For example, compounds of Formula 1-1 (i.e., each Hal can independently be F, Cl, Br, or I) can be reacted with an appropriate amine nucleophile in an appropriate solvent (e.g., 1-butanol) at an appropriate temperature (e.g., ranging from room temperature to 200° C.) for a suitable time (e.g., ranging from several minutes to several days) to generate compounds of Formula 1-2. Alternatively, transition metal (e.g., Pd, Cu, Ni) catalyzed reactions (including, but not limited to, Buchwald, Ullman, Suzuki, Stille, Negishi couplings) of compounds 1-1 and appropriate coupling partners (e.g., primary or secondary amines, nitrogen heterocycles, or heteroaryl boronic acids/esters, trialkyl tin, or zinc reagents) affords compounds of Formula 1-2. Compounds of Formula 1-1 are commercially available, or can be readily synthesized according to methods known by persons skilled in the art. C—N bond forming reactions (e.g., transition metal catalyzed or nucleophilic aromatic substitution) between compounds of Formula 1-2 and hydrazine under appropriate conditions (e.g., in the presence of a palladium catalyst, such as methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (“tBuBrettPhos Pd G3”), and a base, such as Cs₂CO₃ or NaOt-Bu, in an appropriate solvent, such as THF or 1,4-dioxane) generates compounds of Formula 1-3. Reaction of compounds of Formula 1-3 with compounds of Formula 1-4 (e.g., trimethyl orthoformate or triethyl orthoacetate) under appropriate conditions (e.g., in the presence of AcOH) provides compounds of Formula A-1.

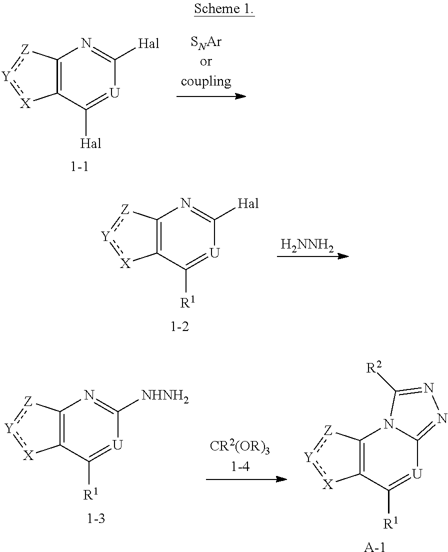

Compounds of Formula I (e.g., compounds of Formula A-2) can also be prepared using the process illustrated in Scheme 2. As depicted in Scheme 2, compounds of Formula 2-1 can be converted into compounds of Formula 2-2 by a number of methods. For example, halogenation of compounds of Formula 2-1 (e.g., via deprotonation with an appropriate base, such as 2,2,6,6-tetramethylpiperidinylmagnesium chloride lithium chloride (“TMPMgCl·LiCl”), followed by addition of an appropriate electrophile, such as 1-chloro-2-iodoethane) followed by a suitable cross-coupling affords compounds of Formula 2-2. Examples of suitable cross-coupling reactions include, but are not limited to, Suzuki (see e.g., Tetrahedron 2002, 58, 9633-9695), Negishi (see e.g., ACS Catalysis 2016, 6, 1540-1552), Stille (see e.g., ACS Catalysis 2015, 5, 3040-3053), Sonogashira (see e.g., Chem. Soc. Rev. 2011, 40, 5084-5121), Buchwald-Hartwig amination (see e.g., Chem. Sci. 2011, 2, 27-50), Cu-catalyzed amination (see e.g., Org. React. 2014, 85, 1-688), among others. Alternatively, compounds of Formula 2-2 can be accessed by conversion of compounds of Formula 2-1 to a carbonyl intermediate (e.g., by deprotonation with an appropriate base, such as TMPMgCl·LiCl, followed by addition of an appropriate electrophile, such as DMF) followed by reaction with a suitable fluorinating reagent (e.g., diethylaminosulfur trifluoride). C—N bond forming reactions (e.g., transition metal catalyzed or nucleophilic aromatic substitution) between compounds of Formula 2-2 and hydrazine under appropriate conditions (e.g., in the presence of a palladacycle precatalyst, such as tBuBrettPhos Pd G3, and a base, such as Cs₂CO₃) generates compounds of Formula 2-2. Reaction of compounds of Formula 2-2 with compounds of Formula 2-3 (e.g., triethyl orthoformate) under appropriate conditions (e.g., in the presence of AcOH) provides compounds of Formula A-2.

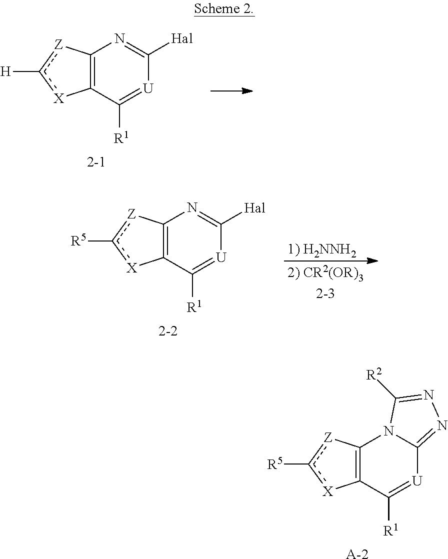

Compounds of Formula 3-8 can be synthesized, for example, according to the process shown in Scheme 3. As depicted in Scheme 3, protection of amino compounds of Formula 3-1 under appropriate conditions (e.g., including, but not limited to, reductive amination reactions with an appropriate aldehyde, such as benzaldehyde, in the presence of a reducing agent, such as sodium triacetoxyborohydride) generates compounds of Formula 3-2. Compounds of Formula 3-1 are commercially available, or can be readily synthesized according to methods known by persons skilled in the art. Amide coupling reactions of compounds of Formula 3-2 with compounds of Formula 3-3 under suitable conditions (e.g., in the presence of a coupling reagent, such as HATU, and a base, such as N-ethyl-N-isopropylpropan-2-amine, in an appropriate solvent, such as N,N-dimethylformamide) affords compounds of Formula 3-4. Deprotection of the tert-butyloxycarbonyl group in compounds of Formula 3-4 under appropriate conditions (e.g., using an acid, such as trifluoroacetic acid), followed by intramolecular cyclization under appropriate conditions (e.g., using a suitable solvent, such as MeOH) provides compounds of Formula 3-5. Reduction of compounds of Formula 3-5 under suitable conditions (e.g., using a reducing agent, such as borane, in a suitable solvent, such as THF) generates compounds of Formula 3-6. Protection of compounds of Formula 3-6 under appropriate conditions (e.g., via reaction with di-tert-butyl dicarbonate in the presence of a base, such as N-ethyl-N-isopropylpropan-2-amine) provides compounds of Formula 3-7. Selective deprotection of PG in compounds of Formula 3-7 (e.g., where PG is a protecting group such as benzyl) under appropriate conditions (e.g., using an appropriate catalyst, such as palladium on carbon, in the presence of hydrogen gas), affords compounds of Formula 3-8.

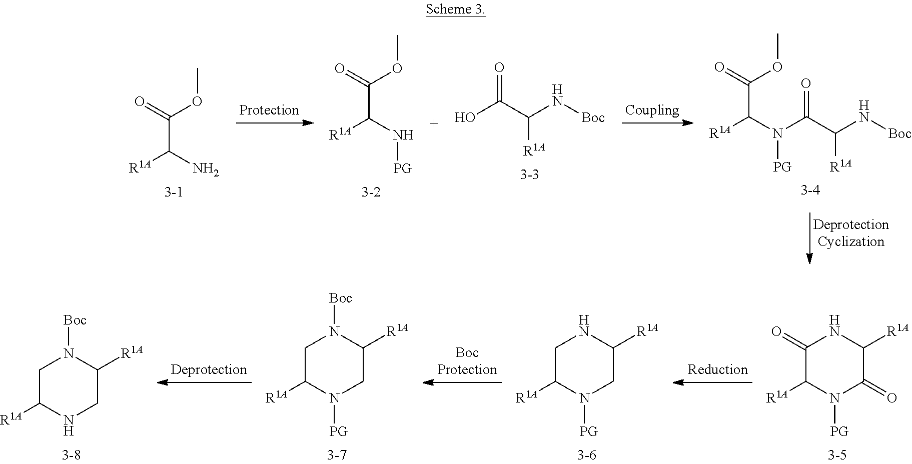

Compounds of Formula 4-4 can be prepared, for example, using the process illustrated in Scheme 4. In the process depicted in Scheme 4, nucleophilic substitution reactions between compounds of Formula 4-1 and compounds of Formula 4-2 under appropriate conditions (e.g., in the presence of a base, such as N-ethyl-N-isopropylpropan-2-amine, in an appropriate solvent, such as CH₃CN) generates compounds of Formula 4-3. Removal of an appropriate protecting group (e.g., wherein PG is a group such as tert-butoxycarbonyl) from compounds of Formula 4-3 under appropriate conditions (e.g., in the presence of an acid, such as HCl or trifluoroacetic acid, in a suitable solvent, such as tetrahydrofuran, 1-4-dioxane, or CH₂Cl₂) affords compounds of Formula 4-4.

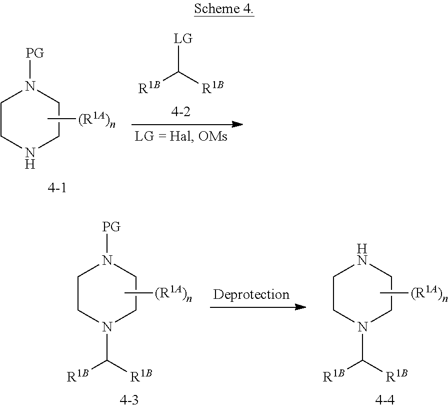

Alternatively, compounds of Formula 4-4 can be prepared, for example, using the process illustrated in Scheme 5. In the process depicted in Scheme 5, amide coupling reactions of compounds of Formula 5-1 with compounds of Formula 5-2 affords compounds of Formula 5-3. Subjection of compounds of Formula 5-3 to reductive alkylation conditions (e.g., through the use of an appropriate transition metal catalyst, such as IrCl(CO)(PPh₃)₂, in the presence of a silane, such as 1,1,3,3-tetramethyldisiloxane, followed by addition of a suitable organometallic reagent, such as a Grignard reagent) affords compounds of Formula 5-4. Removal of an appropriate protecting group (e.g., wherein PG is a group such as tert-butoxycarbonyl) from compounds of Formula 5-4 under appropriate conditions (e.g., in the presence of an acid, such as HCl or trifluoroacetic acid, in a suitable solvent, such as tetrahydrofuran, 1-4-dioxane, or CH₂Cl₂) affords compounds of Formula 4-4.

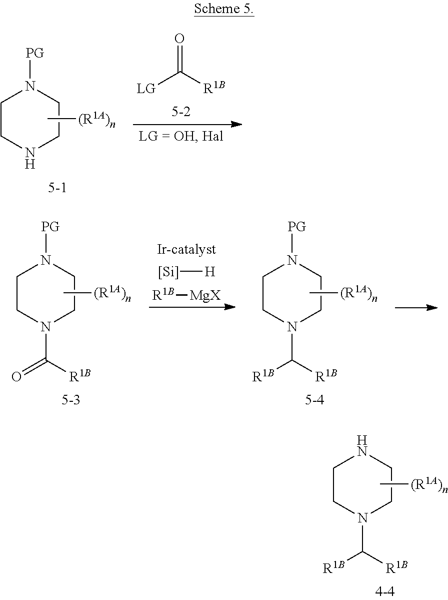

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature”, or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of DGK. Compounds that inhibit DGK are useful in providing a means of preventing the growth or inducing apoptosis of cancer cells. Such compounds are also useful in treating cancer cells exhibiting alterations in diacylglycerol-regulating enzymes and effectors. It is therefore anticipated that the compounds of the disclosure are useful in treating or preventing cancer, such as solid tumors.

In certain embodiments, the disclosure provides a method for treating a DGK-related disorder in a patient in need thereof, comprising the step of administering to said patient a compound of the disclosure, or a pharmaceutically acceptable composition thereof.

The compounds or salts described herein can be selective. By “selective,” it is meant that the compound binds to or inhibits DGKα or DGKζ with greater affinity or potency, respectively, compared to at least one other DGK isoforms, or kinase, etc. In some embodiments, selectivity can be at least about 2-fold, 5-fold, 10-fold, at least about 20-fold, at least about 50-fold, at least about 100-fold, at least about 200-fold, at least about 500-fold or at least about 1000-fold. The compounds of the present disclosure can also be dual antagonists (i.e., inhibitors), e.g. inhibit both DGKα and DGKζ kinases. In some embodiments, the compounds of the invention are selective inhibitors of DGKα (e.g., over one or more other DGK isoforms, or kinase, etc.). In some embodiments, the compounds of the invention are selective inhibitors of DGKζ (e.g., over one or more other DGK isoforms, or kinase, etc.). Selectivity can be measured by methods routine in the art. In some embodiments, selectivity can be tested at the Kₘ ATP concentration of each enzyme. In some embodiments, the selectivity of compounds of the invention can be determined by cellular assays associated with particular DGK kinase activity.

Based on compelling evidence that DGKα and DGK negatively regulate signaling pathways downstream of the T cell receptor, developing DGK inhibitors can boost T cell effector function and inhibit tumor progression. DGK inhibitors can be used to treat, alone or in combination with other therapies, cancers including solid tumors and hematological malignancies, including renal cell carcinoma, mesothelioma, glioblastoma multiforme, colorectal cancer, melanoma, pancreatic cancer (Chen, S. S. et al., Front. Cell Dev. Biol., 2016. 4:130; Gu, J. et al., Oncoimmunol., 2021. 10, e1941566; Jung I.-Y. et al., Cancer Res., 2018. 78:p 4692-4703; Sitaram, P., et al., Int. J Mol. Sci., 2019. 20:p 5821-5848; Wesley, E. M., et al., Immunohorizons, 2018. 2:p 107-118). Furthermore, pharmacological inhibition of DGK provides benefit to control viral infections, and can be used to treatment such viral infections including Coronavirus infection, HIV infection, hepatitis virus infection in preclinical model (Harabuchi, S. et al., Front. Immunol., 2022. 13:1032113).

In addition, DGKα has been shown to enhance esophageal squamous cell carcinoma (ESCC), and human hepatocellular carcinoma (HCC) progression (Chen, J. et al., Oncogene, 2019. 38: p 2533-2550; Takeishi, K. et al., J. Hepatol., 2012. 57:p 77-83), to support colon and breast cancer growth in three-dimensional (3D) culture (Torres-Ayuso, P. et al., Oncotarget, 2014. 5:p 9710-9726), to enhance mammary carcinoma invasiveness (Rainero, E. et al., PLOS ONE, 2014. 9(6): e97144) and promote metastasis of non-small cell lung cancer (NSCLC) (Fu, L. et al., Cancer letters, 2022. 532: 215585) whereas DGKζ has been implicated as a potential oncogene in osteosarcoma proliferation (Yu, W. et al., Front. Oncol., 2019. 8:655) and contributed to enhanced invasion of human metastatic colon cancer cells (Cai, K. et al., BMC Cancer, 2014. 14:208). It has also been reported DGK inhibition has the potential to reduce immunopathology in X-linked lymphoproliferative disease patient (Velnati, S. et al., Eur. J. Med. Chem., 2019. 164: p 378-390; Ruffo, E. et al., Sci. Transl. Med. 2016. 8 (321):321ra7).

In some embodiments, the DGK-related disorder is a solid tumor. Example solid tumors include, but are not limited to, breast cancer, colorectal cancer, gastric cancer, and glioblastoma (see e.g., Cooke & Kazanietz, Sci. Signal, 2022, 15, eabo0264:1-26).

Example cancers associated with alterations in DAG-regulating enzymes and effector include, but are not limited to, uveal melanoma, myelodysplastic syndrome (MDS), angiosarcoma, nodal peripheral T cell lymphoma, adult T-cell leukemia lymphoma (ATLL), cutaneous T-cell lymphoma (CTCL)/Sezary syndrome, chronic lymphocytic leukemia (CLL), breast cancer, gastric cancer, colorectal cancer, oral squamous cell carcinoma (SCC), esophageal SCC, chronic myeloid leukemia (CML), colon cancer, prostate cancer, hepatocellular carcinoma (HCC), blue nevi, NK/T cell lymphoma, glioma, ovarian cancer, liver cancer, melanoma, heptacarcinoma, ostersarcoma, chordiod glioma, pigmented epithelioid melanocytoma, papillary glioneuronal tumor, fibrous histiocytoma, pituitary tumor, thyroid cancer, head and neck SCC, lung cancer, pediatric T-cell acute lymphoblastic leukemia (T-ALL), endometrial cancer, angiolipoma, salivary gland cancer, acute myeloid leukemia (AML), Epstein-Barr virus-associated (EBV)-associated B cell lymphoma, diffuse large B cell lymphoma (DLBCL), and cervical cancer (see e.g., Cooke & Kazanietz, Sci. Signal, 2022, 15, eabo0264:1-26).

In some embodiments, the tumor comprises one or more genetic features selected from high microsatellite instability (MSI-H), mismatch repair deficient (MMRd), and high tumor mutational burden (TMB-H), or any combination thereof.

In some embodiments, the tumor comprises high microsatellite instability (MSI-H), mismatch repair deficient (MMRd), high tumor mutational burden (TMB-H), or mismatch repair deficient (MMRd) and high tumor mutational burden (TMB-H).

In some embodiments, the tumor is identified or has been identified as comprising one or more genetic features selected from high microsatellite instability (MSI-H), mismatch repair deficient (MMRd), and high tumor mutational burden (TMB-H), or any combination thereof.

In some embodiments, the tumor is identified or has been identified comprising high microsatellite instability (MSI-H), mismatch repair deficient (MMRd), high tumor mutational burden (TMB-H), or mismatch repair deficient (MMRd) and high tumor mutational burden (TMB-H).

In some embodiments, the tumor is a tumor with high microsatellite instability (MSI-H). In some embodiments, the tumor is mismatch repair deficient (MMRd). In some embodiments, the tumor is a tumor with high tumor mutational burden (TMB-H). In some embodiments, the tumor is mismatch repair deficient (MMRd) and comprises high tumor mutational burden (TMB-H).

In some embodiments, the cancer is selected from lung cancer, bladder cancer, urothelial cancer, esophageal cancer, stomach cancer, mesothelioma, liver cancer, diffuse large B cell lymphoma, kidney cancer, head and neck cancer, cholangiocarcinoma, cervical cancer, endocervical cancer, melanoma, merkel cell carcinoma (MCC), cutaneous squamous cell carcinoma (CSCC), melanoma, MSI high tumors, ICI sensitive tumors, and viral infection related cancers such as HPV-associated anal cancer, vaginal cancer, vulvar cancer, cervical cancer and oropharyngeal cancer.

In some embodiments, the cancer is selected from lung cancer, bladder cancer, urothelial cancer, esophageal cancer, stomach cancer, mesothelioma, liver cancer, diffuse large B cell lymphoma, kidney cancer, head and neck cancer, cholangiocarcinoma, cervical cancer, endocervical cancer, and melanoma.

In some embodiments, the cancer is selected from non-small cell lung cancer (lung squamous cell carcinoma (LUSC), lung adenocarcinoma (LUAD)), bladder urothelial carcinoma, esophageal carcinoma, stomach adenocarcinoma, mesothelioma, liver hepatocellular carcinoma, diffuse large B cell lymphoma (DLBCL), kidney renal clear cell carcinoma, head and neck squamous cell carcinoma, cholangiocarcinoma, cervical squamous cell carcinoma, endocervical adenocarcinoma, and metastatic melanoma.

In some embodiments, the cancer is a myelodysplastic syndrome. As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × | Dysplasia in ≥10% of cells |
| multilineage dysplasia | 10⁹/L monocytes | in ≥2 hematopoietic lineages, ±15% |
| | | ring sideroblasts, <5% |
| | | blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ±Auer rods, 10% to |
| | monocytes | 19% blasts |
| Myelodysplastic | Cytopenias | Unilineage or no dysplasia but |
| syndrome, unclassified | | characteristic MDS |
| (MDS-U) | | cytogenetics, <5% blasts |
| MDS associated with | Anemia, platelets | Unilineage erythroid. Isolated |
| isolated del(5q) | normal or increased | del(5q), <5% blasts |

In some embodiments, the myelodysplastic syndrome is refractory cytopenia with unilineage dysplasia (RCUID).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a DGK with a compound described herein includes the administration of a compound described herein to an individual or patient, such as a human, having a DGK, as well as, for example, introducing a compound described herein into a sample containing a cellular or purified preparation containing the DGK.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

### I. Immune-Checkpoint Therapies

In some embodiments, DGKα and DGKζ inhibitors provided herein can be used in combination with one or more immune checkpoint inhibitors for the treatment of cancer as described herein.

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, IHPK1, CD137 (also known as 4-1BB), ICOS, A2AR, B7-H3, B7-H4, BTLA, CTLA-4, LAG3, TIM3, TLR (TLR7/8), TIGIT, CD 112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1 BB1).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal or bispecific antibody. In some embodiments, the anti-PD-1 or anti-PD-L1 antibody is nivolumab, pembrolizumab, atezolizumab, durvalumab, avelumab, cemiplimab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550. In some embodiments, the inhibitor of PD-L1 is INCB099280.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012 (INCMGA0012; retifanlimab). In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8119.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, RO7009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGF.beta. receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### II. Cancer Therapies

Cancer cell growth and survival can be impacted by multiple signaling pathways. Thus, it is useful to combine different enzyme/protein/receptor inhibitors, exhibiting different preferences in the targets which they modulate the activities of, to treat such conditions. Examples of agents that may be combined with compounds of the present disclosure, or solid forms or salts thereof, include inhibitors of the PI3K-AKT-mTOR pathway, inhibitors of the Raf-MAPK pathway, inhibitors of JAK-STAT pathway, inhibitors of beta catenin pathway, inhibitors of notch pathway, inhibitors of hedgehog pathway, inhibitors of Pim kinases, and inhibitors of protein chaperones and cell cycle progression. Targeting more than one signaling pathway (or more than one biological molecule involved in a given signaling pathway) may reduce the likelihood of drug-resistance arising in a cell population, and/or reduce the toxicity of treatment.

The compounds of the present disclosure, or solid forms or salts thereof, can be used in combination with one or more other enzyme/protein/receptor inhibitors for the treatment of diseases, such as cancer. Examples of cancers include solid tumors and liquid tumors, such as blood cancers. For example, the compounds of the present disclosure, or solid forms or salts thereof, can be combined with one or more inhibitors of the following kinases for the treatment of cancer: Akt1, Akt2, Akt3, TGF-DR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure, or solid forms or salts thereof, can be combined with one or more of the following inhibitors for the treatment of cancer. Non-limiting examples of inhibitors that can be combined with the compounds of the present disclosure, or solid forms or salts thereof, for treatment of cancers include an FGFR inhibitor (FGFR1, FGFR2, FGFR3 or FGFR4, e.g., AZD4547, BAY1187982, ARQ087, BGJ398, BIBF1120, TKI258, lucitanib, dovitinib, TAS-120, JNJ-42756493, Debiol347, INCB54828, INCB62079 and INCB63904), a JAK inhibitor (JAK1 and/or JAK2, e.g., ruxolitinib, baricitinib or INCB39110), an IDO inhibitor (e.g., epacadostat and NLG919), an LSD1 inhibitor (e.g., GSK2979552, INCB59872 and INCB60003), a TDO inhibitor, a PI3K-delta inhibitor (e.g., INCB50797 and INCB50465), a PI3K-gamma inhibitor such as a PI3K-gamma selective inhibitor, a CSF1R inhibitor (e.g., PLX3397 and LY3022855), a TAM receptor tyrosine kinases (Tyro-3, Axl, and Mer), an angiogenesis inhibitor, an interleukin receptor inhibitor, bromo and extra terminal family members inhibitors (for example, bromodomain inhibitors or BET inhibitors such as OTX015, CPI-0610, INCB54329 and INCB57643) and an adenosine receptor antagonist or combinations thereof. Inhibitors of HDAC such as panobinostat and vorinostat. Inhibitors of c-Met such as onartumzumab, tivantnib, and INC-280. Inhibitors of BTK such as ibrutinib. Inhibitors of mTOR such as rapamycin, sirolimus, temsirolimus, and everolimus. Inhibitors of Raf, such as vemurafenib and dabrafenib. Inhibitors of MEK such as trametinib, selumetinib and GDC-0973. Inhibitors of Hsp90 (e.g., tanespimycin), cyclin dependent kinases (e.g., palbociclib), PARP (e.g., olaparib) and Pim kinases (LGH447, INCB053914 and SGI-1776) can also be combined with compounds of the present disclosure.

Compounds of the present disclosure, or solid forms or salts thereof, can be used in combination with one or more agents for the treatment of diseases such as cancer. In some embodiments, the agent is an alkylating agent, a proteasome inhibitor, a corticosteroid, or an immunomodulatory agent. Examples of an alkylating agent include bendamustine, nitrogen mustards, ethylenimine derivatives, alkyl sulfonates, nitrosoureas and triazenes, uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

The compounds of the present disclosure, or solid forms or salts thereof, can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, CAR (Chimeric antigen receptor) T cell treatment as a booster for T cell activation, oncolytic virotherapy and immunomodulating small molecules, including thalidomide or JAK1/2 inhibitor and the like. The compounds can be administered in combination with one or more anti-cancer drugs, such as a chemotherapeutics. Example chemotherapeutics include any of: abarelix, abiraterone, afatinib, aflibercept, aldesleukin, alemtuzumab, alitretinoin, allopurinol, altretamine, amsacrine, anastrozole, aphidicolon, arsenic trioxide, asparaginase, axitinib, azacitidine, bevacizumab, bexarotene, baricitinib, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, nofetumomab, oserelin, oxaliplatin, paclitaxel, pamidronate, panitumumab, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, ruxolitinib, sorafenib, streptozocin, sunitinib, sunitinib maleate, tamoxifen, tegafur, temozolomide, teniposide, testolactone, thalidomide, thioguanine, thiotepa, topotecan, toremifene, tositumomab, trastuzumab, tretinoin, triptorelin, uracil mustard, valrubicin, vandetanib, vinblastine, vincristine, vindesine, vinorelbine, vorinostat and zoledronate.

Other anti-cancer agent(s) include antibody therapeutics such as trastuzumab (Herceptin), antibodies to costimulatory molecules such as CTLA-4 (e.g., ipilimumab or tremelimumab), 4-1BB, antibodies to PD-1 and PD-L1, or antibodies to cytokines (IL-10, TGF-β, etc.). Examples of antibodies to PD-1 and/or PD-L1 that can be combined with compounds of the present disclosure for the treatment of cancer or infections such as viral, bacteria, fungus and parasite infections include, but are not limited to, nivolumab, pembrolizumab, MPDL3280A, MEDI-4736 and SHR-1210.

Other anti-cancer agents include inhibitors of kinases associated cell proliferative disorder. These kinases include but not limited to Aurora-A, CDK1, CDK2, CDK3, CDK5, CDK7, CDK8, CDK9, ephrin receptor kinases, CHK1, CHK2, SRC, Yes, Fyn, Lck, Fer, Fes, Syk, Itk, Bmx, GSK3, INK, PAK1, PAK2, PAK3, PAK4, PDK1, PKA, PKC, Rsk, and SGK.

Other anti-cancer agents also include those that block immune cell migration such as antagonists to chemokine receptors, including CCR2 and CCR4.

The compounds of the present disclosure, or solid forms or salts thereof, can further be used in combination with one or more anti-inflammatory agents, steroids, immunosuppressants or therapeutic antibodies. The steroids include but are not limited to 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

The compounds of the present disclosure, or solid forms or salts thereof, can also be used in combination with lonafarnib (SCH6636), tipifarnib (R115777), L778123, BMS 214662, tezacitabine (MDL 101731), Sml1, triapine, didox, trimidox and amidox.

The compounds of the disclosure, or salts or solid forms thereof, can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure, or solid forms or salts thereof, can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure, or solid forms or salts thereof, can be used in combination with tumor specific antigen such as heat shock proteins isolated from tumor tissue itself. In some embodiments, the compounds of the present disclosure, or solid forms or salts thereof, can be combined with dendritic cells immunization to activate potent anti-tumor responses.

The compounds of the present disclosure, or solid forms or salts thereof, can be used in combination with bispecific macrocyclic peptides that target Fe alpha or Fe gamma receptor-expressing effectors cells to tumor cells. The compounds of the present disclosure, or solid forms or salts thereof, can also be combined with macrocyclic peptides that activate host immune responsiveness.

The compounds of the present disclosure, or solid forms or salts thereof, can be used in combination with bone marrow transplant for the treatment of a variety of tumors of hematopoietic origin.

Suitable antiviral agents contemplated for use in combination with the compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

When more than one pharmaceutical agent is administered to a patient, they can be administered simultaneously, separately, sequentially, or in combination (e.g., for more than two agents).

In some embodiments, the compounds of the present disclosure, or solid forms or salts thereof, can be used in combination with INCB086550.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating DGK in tissue samples, including human, and for identifying DGK inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes DGK assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms to allow the compound to be deuterated (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, the compound provided herein (e.g., a compound of any of Formulas I-IVa), or a pharmaceutically acceptable salt thereof, comprises at least one deuterium atom.

In some embodiments, the compound provided herein (e.g., a compound of any of Formulas I-IVa), or a pharmaceutically acceptable salt thereof, comprises two or more deuterium atoms.

In some embodiments, the compound provided herein (e.g., a compound of any of Formulas I-IVa), or a pharmaceutically acceptable salt thereof, comprises three or more deuterium atoms.

In some embodiments, for a compound provided herein (e.g., a compound of any of Formulas I-IVa), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro DGK labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind DGK by monitoring its concentration variation when contacting with DGK, through tracking of the labeling. For example, a test compound (labeled) can be evaluated for its ability to reduce binding of another compound which is known to bind to DGK (i.e., standard compound). Accordingly, the ability of a test compound to compete with the standard compound for binding to DGK directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of DGK-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 19×100 mm, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). For purifications using a 30×100 mm column, the flow rate was 60 mL/minute.

pH=10 purifications: Waters XBridge™ Cl₈ 5 μm, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). For purifications using a 30×100 mm column, the flow rate was 60 mL/minute.

### Intermediate 1. (S)-2,6-Dichloro-9-((tetrahydrofuran-2-yl)methyl)-9H-purine

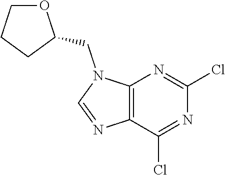 [SMILES: Clc1nc(Cl)c2ncn(C[C@@H]3CCCO3)c2n1]

To a mixture of 2,6-dichloro-9H-purine (27 g, 143 mmol), (S)-(tetrahydrofuran-2-yl)methanol (36.5 g, 357 mmol, BLD Pharmatech BD48351), and triphenylphosphine (94 g, 357 mmol) in THF (714 mL) was added diisopropyl azodicarboxylate (70.3 mL, 357 mmol, Aldrich 225541) and the reaction mixture was stirred at rt for 2 h. Additional (S)-(tetrahydrofuran-2-yl)methanol (1.46 g, 14.3 mmol), triphenylphosphine (3.75 g, 14.3 mmol), and diisopropyl azodicarboxylate (2.82 mL, 14.3 mmol) was added and the reaction mixture was stirred at rt for 1 h. Calcium bromide (140 g, 703 mmol) was added and the reaction mixture was stirred at rt overnight. The mixture was filtered to remove undissolved solids and the filter cake was washed with EtOAc. The filtrate was concentrated in vacuo and the crude residue was purified by flash column chromatography (EtOAc/hexanes). Fractions containing the desired product were combined and concentrated, and the material obtained was triturated with cold Et₂O to afford the desired product (12.5 g, 32% yield) as a white solid. LC-MS calculated for C₁₀H₁₁Cl₂N₄O (M+H)⁺. m/z=273.0. found 273.0.

### Intermediate 2. tert-Butyl (2S,5R)-4-(3,3-difluorocyclobutane-1-carbonyl)-2,5-dimethylpiperazine-1-carboxylate

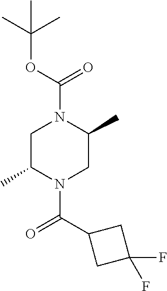 [SMILES: C[C@@H]1CN(C(=O)OC(C)(C)C)[C@@H](C)CN1C(=O)C1CC(F)(F)C1]

A mixture of tert-butyl (2S,5R)-2,5-dimethylpiperazine-1-carboxylate (6.00 g, 28.0 mmol, Combi-Blocks OR-8588) and 3,3-difluorocyclobutane-1-carboxylic acid (4.19 g, 30.8 mmol, Astatech 84107) in MeCN (25 mL) was treated with N,N-diisopropylethylamine (14.7 mL, 84.0 mmol) and HATU (11.2 g, 29.4 mmol, Combi-Blocks OR-0618) and stirred at rt for 30 min. The solvent was removed in vacuo and the residue was diluted with EtOAc and water. The layers were separated and the aqueous layer was extracted with EtOAc. The combined organic layers were washed with brine, dried over MgSO₄, concentrated in vacuo, and purified by flash column chromatography (120 g SiO₂, EtOAc/hexanes) to give the title compound (8.90 g, 96% yield) as a white solid. LC-MS calculated for C₁₂H₁₉F₂N₂O₃(M-C₄H₈+H)⁺: m/z=277.1. found 277.1.

### Intermediate 3. (2R,5S)-1-((3,3-Difluorocyclobutyl)(4-(trifluoromethyl)phenyl)methyl)-2,5-dimethylpiperazine Hydrochloride

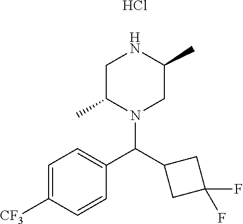 [SMILES: Cl ; C[C@@H]1CN[C@@H](C)CN1C(c1ccc(C(F)(F)F)cc1)C1CC(F)(F)C1]

### Step 1: (4-(Trifluoromethyl)phenyl)magnesium Chloride Lithium Chloride (1.1 M in THF)

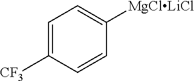

A 1.3 M solution of isopropylmagnesium chloride lithium chloride complex in THF (5.78 mL, 7.52 mmol, Aldrich 656984) was cooled to −78° C. before 1-bromo-4-(trifluoromethyl)benzene (1.14 mL, 8.27 mmol, Aldrich 152692) was added dropwise and the reaction mixture was stirred at −78° C. for 5 min. The reaction mixture was warmed to rt and stirred for an additional 4 h. The mixture obtained was used directly in the next step.

### Step 2: tert-Butyl (2S,5R)-4-((3,3-difluorocyclobutyl)(4-(trifluoromethyl)phenyl)methyl)-2,5-dimethylpiperazine-1-carboxylate

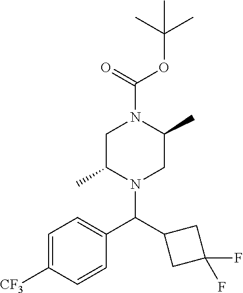 [SMILES: C[C@@H]1CN(C(=O)OC(C)(C)C)[C@@H](C)CN1C(c1ccc(C(F)(F)F)cc1)C1CC(F)(F)C1]

A mixture of tert-butyl (2S,5R)-4-(3,3-difluorocyclobutane-1-carbonyl)-2,5-dimethylpiperazine-1-carboxylate (Intermediate 2, 2.00 g, 6.02 mmol) and chlorocarbonylbis(triphenylphosphine)iridium(I) (0.469 g, 0.602 mmol, Strem 77-0300) in CH₂Cl₂ (10 mL) was treated with 1,1,3,3-tetramethyldisiloxane (2.13 mL, 12.0 mmol, Aldrich 235733) and stirred at rt for 15 min. Immediate gas evolution was observed, and the yellow color of the catalyst became bleached over the course of 15 min. The reaction was cooled to −78° C. and stirred for 5 min before (4-(trifluoromethyl)phenyl)magnesium chloride lithium chloride (Step 1, 6.92 mL, 1.1 M in THF, 7.5 mmol) was added dropwise and the reaction mixture was stirred for an additional 5 min. The reaction mixture was warmed to 0° C. and stirred for 30 min. The mixture was quenched with saturated aqueous NH₄Cl. After warming to rt, the organic layer was removed and the aqueous layer was extracted with CH₂Cl₂. The combined organic layers were dried over MgSO₄ and the filtrate was concentrated in vacuo to afford the desired product as a mixture of diastereomers. The crude material obtained was used directly without further purification. LC-MS calculated for C₂₃H₃₂F₅N₂O₂(M+H)⁺. m/z=463.2. found 463.2.

### Step 3: (2R,5S)-1-((3,3-Difluorocyclobutyl)(4-(trifluoromethyl)phenyl)methyl)-2,5-dimethylpiperazine Hydrochloride

A mixture of tert-butyl (2S,5R)-4-((3,3-difluorocyclobutyl)(4-(trifluoromethyl)phenyl)methyl)-2,5-dimethylpiperazine-1-carboxylate (Step 2) in THF (10 mL) was treated with HCl (4 M in 1,4-dioxane, 10 mL, 40 mmol, Oakwood 094030) and stirred at 60° C. for 1 h. After cooling to rt, the mixture was diluted with diethyl ether and the resulting precipitate was collected by filtration, washed with diethyl ether, and dried under vacuum to afford the desired product (1.50 g, 69% yield over two steps) as a mixture of diastereomers in the form of a white solid. LC-MS calculated for C₁₈H₂₄F₅N₂ (M+H)⁺: m/z=363.2. found 363.2.

### Intermediate 4. (2R,5S)-1-((3,3-Difluorocyclobutyl)(4-(difluoromethyl)-3-fluorophenyl)methyl)-2,5-dimethylpiperazine Hydrochloride

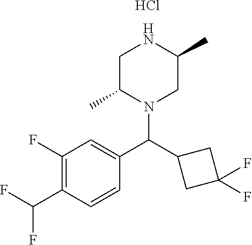 [SMILES: Cl ; C[C@@H]1CN[C@@H](C)CN1C(c1ccc(C(F)F)c(F)c1)C1CC(F)(F)C1]

The title compound was prepared according to the procedures described in Intermediate 3, with 4-bromo-1-(difluoromethyl)-2-fluorobenzene replacing 1-bromo-4-(trifluoromethyl)benzene in Step 1. LC-MS calculated for C₁₈H₂₄F₅N₂ (M+H)⁺: m/z=363.2. found 363.2.

### Intermediate 5. (S)-2,6-Dichloro-8-methyl-9-((tetrahydrofuran-2-yl)methyl)-9H-purine

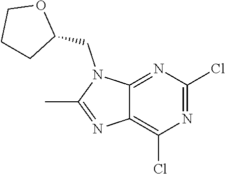 [SMILES: Cc1nc2c(Cl)nc(Cl)nc2n1C[C@@H]1CCCO1]

To a mixture of 2,6-dichloro-8-methylpurine (10.0 g, 49.3 mmol, PharmaBlock PB02898), (S)-(tetrahydrofuran-2-yl)methanol (5.53 g, 54.2 mmol, BLD Pharmatech BD48351), and triphenylphosphine, polymer-bound (100-200 mesh, extent of labeling: ˜1.6 mmol/g loading, Aldrich 93094, 62 g, 99 mmol) in THF (500 mL) was added diisopropyl azodicarboxylate (19.2 mL, 98.7 mmol, Aldrich 225541) and the reaction mixture was stirred at rt for 2 h. The mixture was filtered over Celite and the filtrate was concentrated in vacuo. The crude residue was purified by flash column chromatography (330 g SiO₂, CH₂Cl₂/EtOAc) to afford the desired product (6.8 g, 48% yield) as a white solid. LC-MS calculated for C₁₁H₁₃Cl₂N₄O (M+H)⁺: m/z=287.0. found 287.0.

### Intermediate 6. (2R,5S)-1-((3,3-Difluorocyclobutyl)(4-(difluoromethyl)-2-fluorophenyl)methyl)-2,5-dimethylpiperazine Hydrochloride

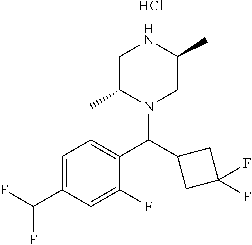 [SMILES: Cl ; C[C@@H]1CN[C@@H](C)CN1C(c1ccc(C(F)F)cc1F)C1CC(F)(F)C1]

The title compound was prepared according to the procedures described in Intermediate 3, with 1-bromo-4-(difluoromethyl)-2-fluorobenzene replacing 1-bromo-4-(trifluoromethyl)benzene in Step 1. LC-MS calculated for C₁₈H₂₄F₅N₂ (M+H)⁺: m/z=363.2. found 363.2.

### Intermediate 7. (2R,5S)-1-((3,3-Difluorocyclobutyl)(2-fluoro-4-(trifluoromethyl)phenyl)methyl)-2,5-dimethylpiperazine Hydrochloride

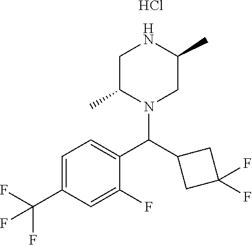 [SMILES: Cl ; C[C@@H]1CN[C@@H](C)CN1C(c1ccc(C(F)(F)F)cc1F)C1CC(F)(F)C1]

The title compound was prepared according to the procedures described in Intermediate 3, with 1-bromo-2-fluoro-4-(trifluoromethyl)benzene replacing 1-bromo-4-(trifluoromethyl)benzene in Step 1. LC-MS calculated for C₁₈H₂₃F₆N₂ (M+H)⁺: m/z=381.2. found 381.4.

### Intermediate 8. (2R,5S)-1-((4-Chloro-2-fluorophenyl)(3,3-difluorocyclobutyl)methyl)-2,5-dimethylpiperazine Hydrochloride

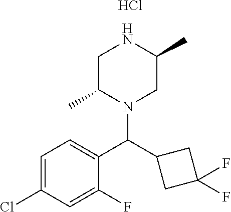 [SMILES: Cl ; C[C@@H]1CN[C@@H](C)CN1C(c1ccc(Cl)cc1F)C1CC(F)(F)C1]

The title compound was prepared according to the procedures described in Intermediate 3, with 4-chloro-2-fluoro-1-iodobenzene replacing 1-bromo-4-(trifluoromethyl)benzene in Step 1. LC-MS calculated for C₁₇H₂₃ClF₃N₂(M+H)⁺: m/z=347.2. found 347.3.

### Intermediate 9. tert-Butyl (2S,5R)-2,5-dimethyl-4-(3-methylbutanoyl)piperazine-1-carboxylate

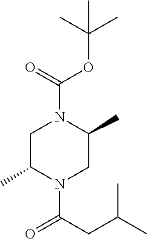 [SMILES: CC(C)CC(=O)N1C[C@H](C)N(C(=O)OC(C)(C)C)C[C@H]1C]

A mixture of tert-butyl (2S,5R)-2,5-dimethylpiperazine-1-carboxylate (2.14 g, 10.0 mmol, Combi-Blocks OR-8588) and N,N-diisopropylethylamine (3.49 mL, 20.00 mmol) in CH₂Cl₂ (33.3 mL) was cooled to 0° C. and isovaleryl chloride (1.463 mL, 12.00 mmol, Aldrich 157422) was added dropwise. The mixture was warmed to room temperature and stirred 30 minutes. Saturated aqueous NaHCO₃ (50 mL) was added and the mixture was stirred vigorously for 15 minutes. The layers were separated and the organic layer was washed with 1 M HCl (50 mL) and brine (50 mL), dried over MgSO₄, and concentrated in vacuo. The title compound (2.92 g, 98% yield) was obtained as a light yellow solid. LC-MS calculated for C₁₆H₃₁N₂O₃ (M+H)⁺: m/z=299.2. found 299.3.

### Intermediate 10. (2R,5S)-1-(1-(4-Chlorophenyl)-3-methylbutyl)-2,5-dimethylpiperazine Hydrochloride

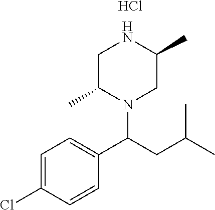 [SMILES: Cl ; CC(C)CC(c1ccc(Cl)cc1)N1C[C@H](C)NC[C@H]1C]

### Step 1: tert-Butyl (2S,5R)-4-(1-(4-chlorophenyl)-3-methylbutyl)-2,5-dimethylpiperazine-1-carboxylate

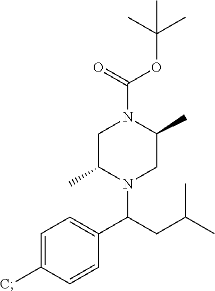

A mixture of tert-butyl (2S,5R)-2,5-dimethyl-4-(3-methylbutanoyl)piperazine-1-carboxylate (Intermediate 9, 1.50 g, 5.03 mmol) and Ir(CO)Cl(PPh₃)₂ (0.118 g, 0.151 mmol, Strem 77-0300) in CH₂Cl₂ (50 mL) was treated with 1,1,3,3-tetramethyldisiloxane (1.78 mL, 10.1 mmol, Aldrich 235733) and stirred 15 minutes at room temperature. After 15 minutes additional Ir(CO)Cl(PPh₃)₂ (0.118 g, 0.151 mmol) and 1,1,3,3-tetramethyldisiloxane (0.89 mL, 5.05 mmol) were added and stirring was continued at room temperature for 15 minutes. The reaction was then cooled to −78° C. and stirred 5 minutes before (4-chlorophenyl)magnesium bromide (1.0 M in diethyl ether, 6.28 mL, 6.28 mmol, Aldrich 262188) was added dropwise. The reaction was stirred an additional 5 minutes, warmed to 0° C. and stirred 30 min. The reaction was quenched with sat. aqueous NH₄Cl. The layers were separated and the aqueous layer was extracted with CH₂Cl₂. The combined organic layers were dried over MgSO₄ and concentrated in vacuo. The material was purified by flash column chromatography (120 g SiO₂, EtOAc/hexanes) to give the title compound (1.74 g, 88% yield) as a single stereoisomer. LC-MS calculated for C₂₂H₃₆ClN₂O₂(M+H)⁺: m/z=395.3. found 395.2.

### Step 2: (2R,5S)-1-(1-(4-chlorophenyl)-3-methylbutyl)-2,5-dimethylpiperazine Hydrochloride

A mixture of tert-butyl (2S,5R)-4-(1-(4-chlorophenyl)-3-methylbutyl)-2,5-dimethylpiperazine-1-carboxylate (1.74 g, 4.41 mmol) in THF (11.0 mL) was treated with HCl (4 M in dioxane, 11.1 mL, 44.2 mmol, Oakwood 094030). The mixture was stirred at stirred at 60° C. for 1 h. The mixture was cooled to room temperature, concentrated in vacuo to approximately half volume, diluted with diethyl ether (10 mL) and hexanes (5 mL), and the precipitate was collected by filtration (washing with 2:1 diethyl ether-hexanes) to give the title compound (1.29 g, 88% yield) as a white solid. LC-MS calculated for C₁₇H₂₈ClN₂ (M+H)⁺: m/z=295.2. found 295.2.

### Intermediate 11. (2R,5S)-2,5-Dimethyl-1-(3-methyl-1-(4-(trifluoromethyl)phenyl)butyl)piperazine Hydrochloride

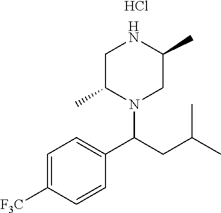 [SMILES: Cl ; CC(C)CC(c1ccc(C(F)(F)F)cc1)N1C[C@H](C)NC[C@H]1C]

The title compound was prepared according to the procedures described for Intermediate 10, with (4-trifluoromethyl)phenyl)magnesium chloride lithium chloride (Intermediate 3, Step 1) replacing (4-chlorophenyl)magnesium bromide. The title compound was isolated as a single stereoisomer. LC-MS calculated for C₁₈H₂₈F₃N₂ (M+H)⁺: m/z=329.2. found 329.2.

### Intermediate 12. (2R,5S)-1-(1-(4-Chloro-3-fluorophenyl)-3-methylbutyl)-2,5-dimethylpiperazine Hydrochloride

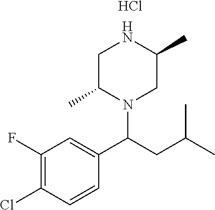 [SMILES: Cl ; CC(C)CC(c1ccc(Cl)c(F)c1)N1C[C@H](C)NC[C@H]1C]

### Step 1: (4-Chloro-3-fluorophenyl)magnesium Chloride Lithium Chloride (0.5M in THF)

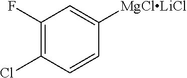

Isopropylmagnesium chloride lithium chloride complex (1.3 M in THF, 4.04 mL, 5.25 mmol, Aldrich 656984) was added dropwise to a solution of 1-chloro-2-fluoro-4-iodobenzene (1.28 g, 5.00 mmol, Oakwood 018374) in THF (10 mL total volume) at −20° C. and the reaction was stirred at this temperature for 30 min. The turbid mixture obtained was used directly in the next step.

### Step 2: tert-Butyl (2S,5R)-4-(1-(4-chloro-3-fluorophenyl)-3-methylbutyl)-2,5-dimethylpiperazine-1-carboxylate

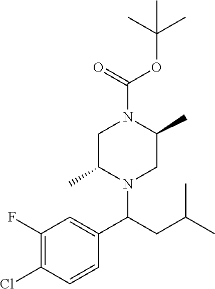 [SMILES: CC(C)CC(c1ccc(Cl)c(F)c1)N1C[C@H](C)N(C(=O)OC(C)(C)C)C[C@H]1C]

The title compound was prepared according to the procedure described in Step 1 for Intermediate 10, with (4-chloro-3-fluorophenyl)magnesium chloride lithium chloride (0.5 M in THF, Step 1) replacing (4-chlorophenyl)magnesium bromide. The title compound was isolated as a single stereoisomer. LC-MS calculated for C₂₂H₃₅ClFN₂O₂ (M+H)⁺. m/z=413.2. found 413.3.

### Step 3: (2R,5S)-1-(1-(4-Chloro-3-fluorophenyl)-3-methylbutyl)-2,5-dimethylpiperazine Hydrochloride

The title compound was prepared according to the procedure described in Step 2 for Intermediate 10, with tert-butyl (2S,5R)-4-(1-(4-chloro-3-fluorophenyl)-3-methylbutyl)-2,5-dimethylpiperazine-1-carboxylate (Step 2) replacing tert-butyl (2S,5R)-4-(1-(4-chlorophenyl)-3-methylbutyl)-2,5-dimethylpiperazine-1-carboxylate. LC-MS calculated for C₁₇H₂₇ClFN₂ (M+H)⁺: m/z=313.2. found 313.2.

### Intermediate 13. tert-Butyl (2S,5R)-4-(cyclopropanecarbonyl)-2,5-dimethylpiperazine-1-carboxylate

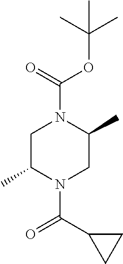 [SMILES: C[C@@H]1CN(C(=O)OC(C)(C)C)[C@@H](C)CN1C(=O)C1CC1]

The title compound was prepared according to the procedure described for Intermediate 9, with cyclopropanecarbonyl chloride (Aldrich C116807) replacing isovaleryl chloride. LC-MS calculated for C₁₁H₁₉N₂O₃ (M-C₄H₈+H)⁺: m/z=227.1. found 227.2.

### Intermediate 14. tert-Butyl (2S,5R)-4-((4-chlorophenyl)(cyclopropyl)methyl)-2,5-dimethylpiperazine-1-carboxylate

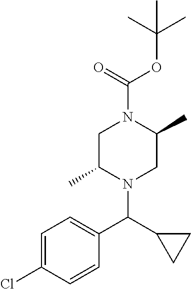 [SMILES: C[C@@H]1CN(C(=O)OC(C)(C)C)[C@@H](C)CN1C(c1ccc(Cl)cc1)C1CC1]

The title compound was prepared according to the procedure described in Step 1 for Intermediate 10, with tert-butyl (2S,5R)-4-(cyclopropanecarbonyl)-2,5-dimethylpiperazine-1-carboxylate (Intermediate 13) replacing tert-butyl (2S,5R)-2,5-dimethyl-4-(3-methylbutanoyl)piperazine-1-carboxylate. The title compound was isolated as a mixture of diastereomers. LC-MS calculated for C₂₁H₃₂ClN₂O₂(M+H)⁺: m/z=379.2. found 379.2.

### Intermediate 15. tert-Butyl (2S,5R)-4-((S)-2,2-difluorocyclopropane-1-carbonyl)-2,5-dimethylpiperazine-1-carboxylate

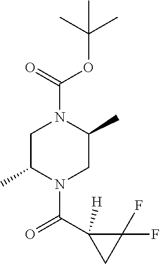 [SMILES: C[C@@H]1CN(C(=O)OC(C)(C)C)[C@@H](C)CN1C(=O)[C@@H]1CC1(F)F]

A mixture of tert-butyl (2S,5R)-2,5-dimethylpiperazine-1-carboxylate (2.14 g, 10.0 mmol Combi-Blocks OR-8588) and (S)-2,2-difluorocyclopropane-1-carboxylic acid (1.22 g, 10.00 mmol, AstaTech P15788) in MeCN (33 mL) was treated with N,N-diisopropylethylamine (3.49 mL, 20.0 mmol) and HATU (3.99 g, 10.5 mmol, Combi-Blocks OR-0618) and stirred at rt overnight. The solvent was removed in vacuo and the crude residue was diluted with EtOAc and water. The layers were separated and the aqueous layer was extracted with EtOAc. The combined organic layers were washed with brine, dried over MgSO₄, concentrated in vacuo, and purified by flash column chromatography (40 g SiO₂, EtOAc/hexanes) to give the title compound (2.82 g, 84% yield) as a white solid. LC-MS calculated for C₁₁H₁₇F₂N₂O₃(M-C₄H₈+H)⁺: m/z=263.1. found 263.2.

### Intermediate 16. (2R,5S)-1-((4-Chlorophenyl)((S)-2,2-difluorocyclopropyl)methyl)-2,5-dimethylpiperazine Hydrochloride

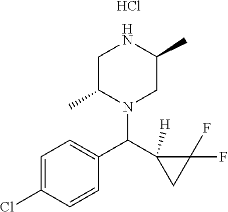 [SMILES: Cl ; C[C@@H]1CN[C@@H](C)CN1C(c1ccc(Cl)cc1)[C@@H]1CC1(F)F]

The title compound was prepared according to the procedures described for Intermediate 10, with tert-butyl (2S,5R)-4-((S)-2,2-difluorocyclopropane-1-carbonyl)-2,5-dimethylpiperazine-1-carboxylate (Intermediate 15) replacing tert-butyl (2S,5R)-2,5-dimethyl-4-(3-methylbutanoyl)piperazine-1-carboxylate. The title compound was isolated as a single stereoisomer. LC-MS calculated for C₁₆H₂₂ClF₂N₂(M+H)⁺: m/z=315.1. found 315.2.

### Intermediate 17. tert-Butyl (2S,5R)-4-(((S)-2,2-difluorocyclopropyl)(4-(trifluoromethyl)phenyl)methyl)-2,5-dimethylpiperazine-1-carboxylate

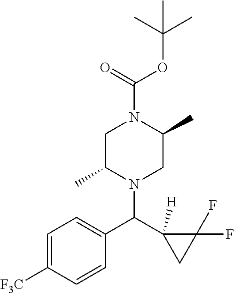 [SMILES: C[C@@H]1CN(C(=O)OC(C)(C)C)[C@@H](C)CN1C(c1ccc(C(F)(F)F)cc1)[C@@H]1CC1(F)F]

The title compound was prepared according to the procedure described in Step 1 for Intermediate 10, with tert-butyl (2S,5R)-4-((S)-2,2-difluorocyclopropane-1-carbonyl)-2,5-dimethylpiperazine-1-carboxylate (Intermediate 15) replacing tert-butyl (2S,5R)-2,5-dimethyl-4-(3-methylbutanoyl)piperazine-1-carboxylate and (4-trifluoromethyl)phenyl)magnesium chloride lithium chloride (Intermediate 3, Step 1) replacing (4-chlorophenyl)magnesium bromide. The title compound was isolated as a single stereoisomer. LC-MS calculated for C₂₂H₃₀F₅N₂O₂(M+H)⁺: m/z=449.2. found 449.3.

### Intermediate 18. tert-Butyl (2S,5R)-4-((R)-2,2-difluorocyclopropane-1-carbonyl)-2,5-dimethylpiperazine-1-carboxylate

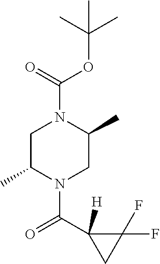 [SMILES: C[C@@H]1CN(C(=O)OC(C)(C)C)[C@@H](C)CN1C(=O)[C@H]1CC1(F)F]

The title compound was prepared according to the procedure described for Intermediate 15, with (R)-2,2-difluorocyclopropane-1-carboxylic acid (AstaTech P17160) replacing (S)-2,2-difluorocyclopropane-1-carboxylic acid. LC-MS calculated for C₁₁H₁₇F₂N₂O₃(M-C₄H₈+H)⁺: m/z=263.1. found 263.2.

### Intermediate 19. tert-Butyl (2S,5R)-4-((4-chlorophenyl)((R)-2,2-difluorocyclopropyl)methyl)-2,5-dimethylpiperazine-1-carboxylate

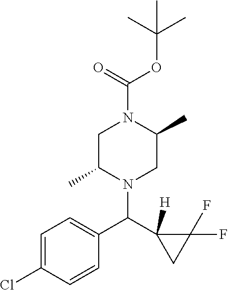 [SMILES: C[C@@H]1CN(C(=O)OC(C)(C)C)[C@@H](C)CN1C(c1ccc(Cl)cc1)[C@H]1CC1(F)F]

The title compound was prepared according to the procedure described in Step 1 for Intermediate 10, with tert-butyl (2S,5R)-4-((R)-2,2-difluorocyclopropane-1-carbonyl)-2,5-dimethylpiperazine-1-carboxylate (Intermediate 18) replacing tert-butyl (2S,5R)-2,5-dimethyl-4-(3-methylbutanoyl)piperazine-1-carboxylate. The title compound was isolated as a single stereoisomer. LC-MS calculated for C₂₁H₃₀ClF₂N₂O₂ (M+H)⁺: m/z=415.2. found 415.2.

### Intermediate 20. tert-Butyl (2S,5R)-2,5-dimethyl-4-(4,4,4-trifluoro-3-methylbutanoyl)piperazine-1-carboxylate

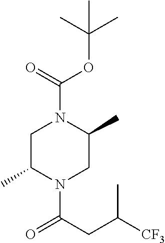 [SMILES: CC(CC(=O)N1C[C@H](C)N(C(=O)OC(C)(C)C)C[C@H]1C)C(F)(F)F]

The title compound was prepared according to the procedure described for Intermediate 15, with 3-(trifluoromethyl)butyric acid (Alfa Aesar L12160) replacing (S)-2,2-difluorocyclopropane-1-carboxylic acid. The title compound was isolated as a mixture of diastereomers. LC-MS calculated for C₁₂H₂₀F₃N₂O₃(M-C₄H₈+H)⁺: m/z=297.1. found 297.2.

### Intermediate 21. tert-Butyl (2S,5R)-4-(1-(4-chlorophenyl)-4,4,4-trifluoro-3-methylbutyl)-2,5-dimethylpiperazine-1-carboxylate

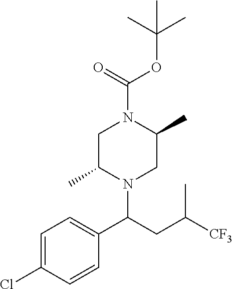 [SMILES: CC(CC(c1ccc(Cl)cc1)N1C[C@H](C)N(C(=O)OC(C)(C)C)C[C@H]1C)C(F)(F)F]

The title compound was prepared according to the procedure described in Step 1 for Intermediate 10, with tert-butyl (2S,5R)-2,5-dimethyl-4-(4,4,4-trifluoro-3-methylbutanoyl)piperazine-1-carboxylate (Intermediate 20) replacing tert-butyl (2S,5R)-2,5-dimethyl-4-(3-methylbutanoyl)piperazine-1-carboxylate. The title compound was isolated as a mixture of diastereomers. LC-MS calculated for C₂₂H₃₃ClF₃N₂O₂ (M+H)⁺: m/z=449.2. found 449.2.

### Intermediate 22. (2R,5S)-1-(1-(4-Chloro-3-fluorophenyl)-4,4,4-trifluoro-3-methylbutyl)-2,5-dimethylpiperazine Hydrochloride

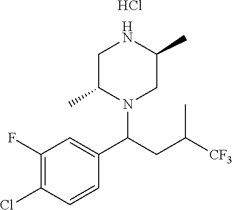 [SMILES: Cl ; CC(CC(c1ccc(Cl)c(F)c1)N1C[C@H](C)NC[C@H]1C)C(F)(F)F]

The title compound was prepared according to the procedures described for Intermediate 10, with tert-butyl (2S,5R)-2,5-dimethyl-4-(4,4,4-trifluoro-3-methylbutanoyl)piperazine-1-carboxylate (Intermediate 20) replacing tert-butyl (2S,5R)-2,5-dimethyl-4-(3-methylbutanoyl)piperazine-1-carboxylate and (4-chloro-3-fluorophenyl)magnesium chloride lithium chloride (Intermediate 12, Step 1) replacing (4-chlorophenyl)magnesium bromide. The title compound was isolated as a mixture of diastereomers. LC-MS calculated for C₁₇H₂₄ClF₄N₂(M+H)⁺. m/z=367.2. found 367.1.

### Intermediate 23. Bis(5-(trifluoromethyl)pyridin-2-yl)methyl Methanesulfonate

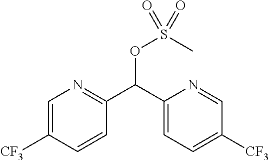 [SMILES: CS(=O)(=O)OC(c1ccc(C(F)(F)F)cn1)c1ccc(C(F)(F)F)cn1]

### Step 1: Bis(5-(trifluoromethyl)pyridin-2-yl)methanol

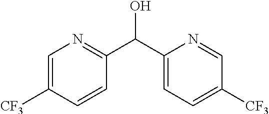 [SMILES: OC(c1ccc(C(F)(F)F)cn1)c1ccc(C(F)(F)F)cn1]

To a mixture of 2-bromo-5-(trifluoromethyl)pyridine (2.64 g, 11.7 mmol, Aldrich 661120) in Et₂O (47 mL) at 0° C. was added isopropylmagnesium chloride lithium chloride complex (1.3 M in THF, 9.43 mL, 12.3 mmol, Aldrich 656984) dropwise over 5 min. The light orange solution became dark red over time. After stirring at 0° C. for 2 h, a solution of 5-(trifluoromethyl)picolinaldehyde (2.04 g, 11.7 mmol, Combi-Blocks PY-1433) in Et₂O (10 mL) was added. A precipitate formed immediately. The reaction mixture was stirred at 0° C. for 5 min, then quenched with sat. aq. NH₄Cl. After warming to rt, the layers were separated. The organic layer was removed and the aqueous layer was extracted with EtOAc. The combined organic layers were dried over MgSO₄ and concentrated in vacuo. The crude residue was purified by flash column chromatography (40 g SiO₂, EtOAc/hexanes) to give the title compound (1.98 g, 60% yield) as an orange solid. LC-MS calculated for C₁₃H₉F₆N₂O (M+H)⁺: m/z=323.1. found 323.1.

### Step 2: Bis(5-(trifluoromethyl)pyridin-2-yl)methyl Methanesulfonate

A mixture of bis(5-(trifluoromethyl)pyridin-2-yl)methanol (1.98 g, 6.14 mmol) and N,N-diisopropylethylamine (3.22 mL, 18.42 mmol) in CH₂Cl₂ (12.3 mL) was cooled to 0° C. Methanesulfonyl chloride (0.718 mL, 9.21 mmol) was added dropwise and the reaction mixture was stirred at 0° C. for 1 h. The mixture was diluted with water and after warming to rt the layers were separated. The organic layer was removed and the aqueous layer was extracted with CH₂Cl₂. The combined organic layers were dried over MgSO₄ and concentrated in vacuo. The crude residue was purified by flash column chromatography (40 g SiO₂, EtOAc/hexanes) to give the title compound (2.33 g, 95% yield) as an orange solid. LC-MS calculated for C₁₄H₁₁F₆N₂O₃S (M+H)⁺: m/z=401.0. found 401.1.

### Intermediate 24. (2R,5S)-1-(Bis(5-(trifluoromethyl)pyridin-2-yl)methyl)-2,5-dimethylpiperazine Dihydrochloride

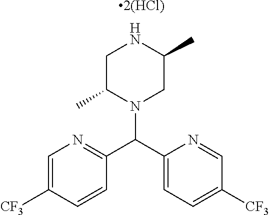

### Step 1: tert-Butyl (2S,5R)-4-(bis(5-(trifluoromethyl)pyridin-2-yl)methyl)-2,5-dimethylpiperazine-1-carboxylate

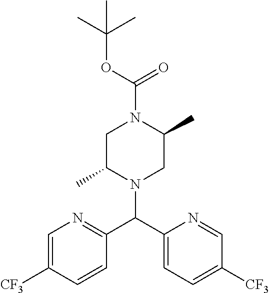 [SMILES: C[C@@H]1CN(C(=O)OC(C)(C)C)[C@@H](C)CN1C(c1ccc(C(F)(F)F)cn1)c1ccc(C(F)(F)F)cn1]

A mixture of tert-butyl (2S,5R)-2,5-dimethylpiperazine-1-carboxylate (1.32 g, 6.14 mmol, Combi-Blocks OR-8588), bis(5-(trifluoromethyl)pyridin-2-yl)methyl methanesulfonate (Intermediate 23, 2.33 g, 5.81 mmol) and N,N-diisopropylethylamine (3.22 mL, 18.2 mmol) in MeCN (30 mL) was stirred at 85° C. overnight. After cooling to rt, the reaction mixture was concentrated in vacuo. The crude residue was taken up in EtOAc and sat. aq. NaHCO₃ was added. The layers were separated and the aqueous layer was extracted with EtOAc. The combined organic layers were washed with brine, dried over Na₂SO₄, and concentrated in vacuo. The crude residue was purified by flash column chromatography (40 g SiO₂, EtOAc/hexanes) to give the title compound as an orange oil. LC-MS calculated for C₂₄H₂₉F₆N₄O₂(M+H)⁺: m/z=519.2. found 519.2.

### Step 2: (2R,5S)-1-(Bis(5-(trifluoromethyl)pyridin-2-yl)methyl)-2,5-dimethylpiperazine Dihydrochloride

To a mixture of tert-butyl (2S,5R)-4-(bis(5-(trifluoromethyl)pyridin-2-yl)methyl)-2,5-dimethylpiperazine-1-carboxylate (Step 1) in THF (15 mL) was added HCl (4 M in 1,4-dioxane, 15.4 mL, 61.4 mmol) and the reaction mixture was stirred at 60° C. for 1 h. After cooling to rt, the mixture was diluted with diethyl ether (100 mL). The resulting precipitate was collected by filtration, washed with diethyl ether, and dried under vacuum to give the title compound (1.24 g, 43% yield over two steps) as a green solid. LC-MS calculated for C₁₉H₂₁F₆N₄ (M+H)⁺: m/z=419.2. found 419.3.

### Intermediate 25. Bis(5-chloropyridin-2-yl)methyl Methanesulfonate

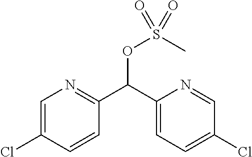 [SMILES: CS(=O)(=O)OC(c1ccc(Cl)cn1)c1ccc(Cl)cn1]

The title compound was prepared according to the procedures described for Intermediate 23, with 2-bromo-5-chloropyridine (Combi-Blocks PY-7032) replacing 2-bromo-5-(trifluoromethyl)pyridine and 5-chloropiconlinaldehyde (Enamine EN300-383915) replacing 5-(trifluoromethyl)picolinaldehyde. LC-MS calculated for C₁₂H₁₁Cl₂N₂O₃S (M+H)⁺: m/z=333.0. found 333.1.

### Intermediate 26. (2R,5S)-1-(Bis(5-chloropyridin-2-yl)methyl)-2,5-dimethylpiperazine Dihydrochloride

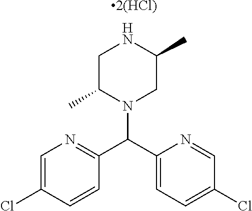

The title compound was prepared according to the procedures described for Intermediate 24, with bis(5-chloropyridin-2-yl)methyl methanesulfonate (Intermediate 25) replacing bis(5-(trifluoromethyl)pyridin-2-yl)methyl methanesulfonate. LC-MS calculated for C₁₇H₂₁Cl₂N₄(M+H)⁺: m/z=351.1. found 351.1.

### Intermediate 27. tert-Butyl (2S,5R)-4-(5-chloropicolinoyl)-2,5-dimethylpiperazine-1-carboxylate

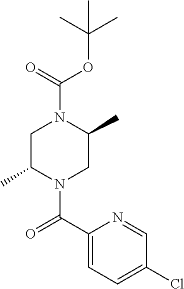 [SMILES: C[C@@H]1CN(C(=O)OC(C)(C)C)[C@@H](C)CN1C(=O)c1ccc(Cl)cn1]

The title compound was prepared according to the procedure described for Intermediate 15, with 5-chloropicolinic acid (Enamine EN300-80716) replacing (S)-2,2-difluorocyclopropane-1-carboxylic acid. LC-MS calculated for C₁₃H₁₇ClN₃O₃ (M-C₄H₈+H)⁺: m/z=298.1. found 298.1.

### Intermediate 28. (2R,5S)-1-((4-Chlorophenyl)(5-chloropyridin-2-yl)methyl)-2,5-dimethylpiperazine Hydrochloride

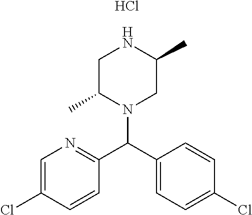 [SMILES: Cl ; C[C@@H]1CN[C@@H](C)CN1C(c1ccc(Cl)cc1)c1ccc(Cl)cn1]

The title compound was prepared according to the procedures described for Intermediate 10, with tert-butyl (2S,5R)-4-(5-chloropicolinoyl)-2,5-dimethylpiperazine-1-carboxylate (Intermediate 27) replacing tert-butyl (2S,5R)-2,5-dimethyl-4-(3-methylbutanoyl)piperazine-1-carboxylate. The title compound was isolated as a mixture of diastereomers. LC-MS calculated for C₁₈H₂₂Cl₂N₃(M+H)⁺: m/z=350.1. found 350.2.

### Intermediate 29. tert-Butyl (2S,5R)-4-isobutyryl-2,5-dimethylpiperazine-1-carboxylate

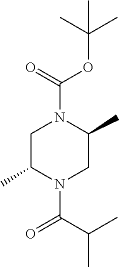 [SMILES: CC(C)C(=O)N1C[C@H](C)N(C(=O)OC(C)(C)C)C[C@H]1C]

A mixture of isobutyryl chloride (21 ml, 200 mmol) in CH₂Cl₂ (400 mL) was charged with tert-butyl (2S,5R)-2,5-dimethylpiperazine-1-carboxylate (42.9 g, 200 mmol) at 0° C. Following that, triethylamine (84 mL, 600 mmol) was added slowly at the same temperature. The resulting heterogeneous mixture was allowed to gradually warm to rt and stirred for 1 h. The mixture was washed with 1 M HCl and saturated sodium bicarbonate solution. After drying over sodium sulfate, the mixture was concentrated under reduced pressure to give the desired product (54.5 g, 96% yield) as a white solid. The material obtained was used directly without further purification. LC-MS calculated for C₁₁H₂₁N₂O₃ (M-C₄H₈+H)⁺. m/z=229.2. found 229.2.

### Intermediate 30. (2R,5S)-1-(1-(4-Chloro-3-fluorophenyl)-2-methylpropyl)-2,5-dimethylpiperazine Dihydrochloride

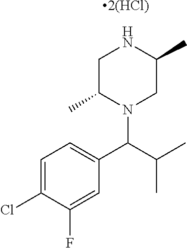

### Step 1: (4-Chloro-3-fluorophenyl)magnesium Chloride Lithium Chloride (0.75 M in THF)

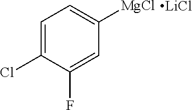

A 1.3 M solution of isopropylmagnesium chloride lithium chloride complex in THF (7.59 mL, 9.87 mmol, Aldrich 656984) was cooled to −78° C. before a mixture of 1-chloro-2-fluoro-4-iodobenzene (2.3 g, 8.97 mmol) in dry THF (4.48 mL) was added dropwise and the reaction mixture was stirred at −78° C. for 5 min. The reaction mixture was warmed to rt and stirred for an additional 4 h. The mixture obtained was used directly in the next step.

### Step 2: tert-Butyl (2S,5R)-4-(1-(4-chloro-3-fluorophenyl)-2-methylpropyl)-2,5-dimethylpiperazine-1-carboxylate

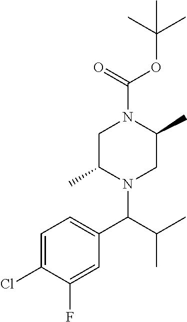 [SMILES: CC(C)C(c1ccc(Cl)c(F)c1)N1C[C@H](C)N(C(=O)OC(C)(C)C)C[C@H]1C]

A mixture of tert-butyl (2S,5R)-4-isobutyryl-2,5-dimethylpiperazine-1-carboxylate (Intermediate 29, 2.00 g, 7.03 mmol) and chlorocarbonylbis(triphenylphosphine)iridium(I) (549 mg, 0.703 mmol, Strem 77-0300) in CH₂Cl₂ (23.4 mL) was treated with 1,1,3,3-tetramethyldisiloxane (2.49 mL, 14.1 mmol, Aldrich 235733) and stirred at rt for 20 min. The reaction mixture was cooled to −78° C. and stirred for 5 min before (4-chloro-3-fluorophenyl)magnesium chloride lithium chloride (11.7 mL, 0.75 M in THF, 3.52 mmol, Step 1) was added dropwise and the reaction mixture was stirred for an additional 5 min before warming to rt and stirring for 1 h. The mixture was quenched with saturated aqueous NH₄Cl and the layers were separated. The organic layer was removed and the aqueous layer was extracted with CH₂Cl₂. The combined organic layers were dried over MgSO₄ and the filtrate was concentrated in vacuo to afford the desired product as a mixture of diastereomers. The crude material obtained was used directly without further purification. LC-MS calculated for C₂₁H₃₃ClFN₂O₂(M+H)⁺: m/z=399.2. found 399.2.

### Step 3: (2R,5S)-1-(1-(4-Chloro-3-fluorophenyl)-2-methylpropyl)-2,5-dimethylpiperazine dihydrochloride

A mixture of tert-butyl (2S,5R)-4-(1-(4-chloro-3-fluorophenyl)-2-methylpropyl)-2,5-dimethylpiperazine-1-carboxylate (Step 2) in a 4 M solution of HCl in 1,4-dioxane (21.1 mL, 84.0 mmol) was stirred at 50° C. for 1 h. After cooling to rt, the mixture was diluted with Et₂O/hexanes (2:1) and slurried at rt for 30 min. The resulting precipitate was collected by filtration, washed with diethyl ether, and dried under vacuum to afford the desired product (2.3 g, 88% yield over two steps) as a mixture of diastereomers in the form of a white solid. LC-MS calculated for C₁₆H₂₅ClFN₂ (M+H)⁺: m/z=299.2. found 299.2.

### Intermediate 31. (2R,5S)-1-(1-(3-Fluoro-4-methoxyphenyl)-2-methylpropyl)-2,5-dimethylpiperazine Dihydrochloride

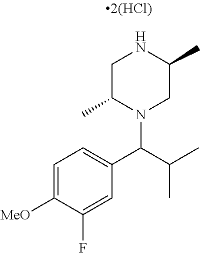 [SMILES: COc1ccc(C(C(C)C)N2C[C@H](C)NC[C@H]2C)cc1F]

The title compound was prepared according to the procedures described for Intermediate 30, with 4-bromo-2-fluoro-1-methoxybenzene replacing 1-chloro-2-fluoro-4-iodobenzene in Step 1. LC-MS calculated for C₁₇H₂₈FN₂O (M+H)⁺: m/z=295.2. found 295.3.

### Intermediate 32. tert-Butyl (2S,5R)-4-(4-(difluoromethyl)-2-fluorobenzoyl)-2,5-dimethylpiperazine-1-carboxylate

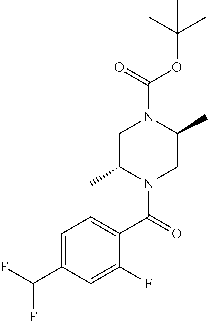 [SMILES: C[C@@H]1CN(C(=O)OC(C)(C)C)[C@@H](C)CN1C(=O)c1ccc(C(F)F)cc1F]

To a mixture of tert-butyl (2S,5R)-2,5-dimethylpiperazine-1-carboxylate (1.24 g, 5.79 mmol) and 4-(difluoromethyl)-2-fluorobenzoic acid (1.0 g, 5.26 mmol) in CH₃CN (7.0 mL) was added HATU (2.20 g, 5.79 mmol) followed by N-ethyl-N-isopropylpropan-2-amine (1.8 mL, 10.5 mmol) and the reaction mixture was stirred at rt overnight. The mixture was diluted with EtOAc and washed with water. After phase separation the organic layer was removed and the aqueous layer was extracted with EtOAc. The combined organic layers were dried over MgSO₄, concentrated, and the crude residue was purified by flash column chromatography (SiO₂, EtOAc/hexanes) to afford the desired product (1.85 g, 91% yield). LC-MS calculated for C₁₅H₁₈F₃N₂O₃(M-C₄H₈+H)⁺. m/z=331.1. found 331.1.

### Intermediate 33. (2R,5S)-1-(1-(4-(Difluoromethyl)-2-fluorophenyl)-2-methylpropyl)-2,5-dimethylpiperazine Dihydrochloride

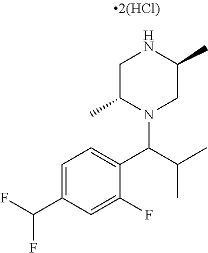 [SMILES: CC(C)C(c1ccc(C(F)F)cc1F)N1C[C@H](C)NC[C@H]1C]

### Step 1. tert-Butyl (2S,5R)-4-(1-(4-(difluoromethyl)-2-fluorophenyl)-2-methylpropyl)-2,5-dimethylpiperazine-1-carboxylate

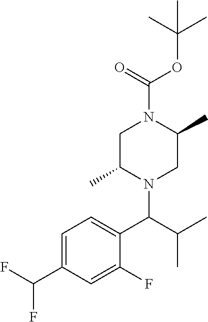 [SMILES: CC(C)C(c1ccc(C(F)F)cc1F)N1C[C@H](C)N(C(=O)OC(C)(C)C)C[C@H]1C]

To a mixture of tert-butyl (2S,5R)-4-(4-(difluoromethyl)-2-fluorobenzoyl)-2,5-dimethylpiperazine-1-carboxylate (Intermediate 32, 1.0 g, 2.59 mmol) and chlorocarbonylbis(triphenylphosphine)iridium(I) (202 mg, 0.259 mmol, Strem 77-0300) in CH₂Cl₂ (8.6 mL) under a nitrogen atmosphere was added 1,1,3,3-tetramethyldisiloxane (0.82 mL, 5.18 mmol, Aldrich 235733). The reaction mixture was stirred for 20 min at rt. The reaction mixture was cooled to −78° C. and stirred for 5 min before a 2.0 M solution of isopropylmagnesium chloride in THF (2.50 mL, 3.23 mmol, Aldrich 230111) was added dropwise and the mixture was stirred at −78° C. for an additional 5 min. The reaction mixture was warmed to 0° C. and stirred for 1 h before being quenched with saturated aqueous NH₄Cl. The layers were separated and the aqueous layer was extracted with CH₂Cl₂. The combined organic layers were dried over Na₂SO₄ and concentrated under reduced pressure to afford the desired product as a mixture of diastereomers. The crude material obtained was used directly without further purification. LC-MS calculated for C₂₂H₃₄F₃N₂O₂(M+H)⁺: m/z=415.3. found 415.3.

### Step 2. (2R,5S)-1-(1-(4-(Difluoromethyl)-2-fluorophenyl)-2-methylpropyl)-2,5-dimethylpiperazine Dihydrochloride

To a mixture of tert-butyl (2S,5R)-4-(1-(4-(difluoromethyl)-2-fluorophenyl)-2-methylpropyl)-2,5-dimethylpiperazine-1-carboxylate (Step 1) in THF (8.6 mL) was added a 4 molar solution of HCl in 1,4-dioxane (7.77 mL, 31.1 mmol) and the reaction mixture was stirred at 50° C. for 1 h. After cooling to rt, the reaction mixture was diluted with Et₂O/hexanes (2:1) and slurried for 30 min. The solid precipitate was collected via filtration, washed with Et₂O and hexanes, and dried under vacuum to afford the desired product (0.875 g) as a mixture of diastereomers in the form of a white solid. LC-MS calculated for C₁₇H₂₆F₃N₂ (M+H)⁺: m/z=315.2. found 315.2.

### Intermediate 34. tert-Butyl (2S,5R)-4-(4-bromo-2-fluorobenzoyl)-2,5-dimethylpiperazine-1-carboxylate

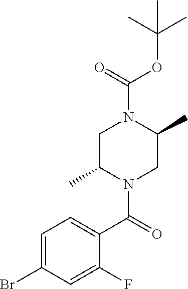 [SMILES: C[C@@H]1CN(C(=O)OC(C)(C)C)[C@@H](C)CN1C(=O)c1ccc(Br)cc1F]

A mixture of tert-butyl (2S,5R)-2,5-dimethylpiperazine-1-carboxylate (1.81 g, 8.42 mmol, Combi-Blocks OR-8588) in CH₂Cl₂ (8.4 mL) was cooled to 0° C. before 4-bromo-2-fluorobenzoyl chloride (2.0 g, 8.42 mmol) was added followed by triethylamine (3.52 mL, 25.3 mmol) and the reaction mixture was allowed to warm to rt and stirred overnight. The mixture was transferred to a separatory funnel and the organic phase was washed with 1 M HCl (aq) and brine. The organic phase was dried over MgSO₄ and concentrated under reduced pressure to afford the desired product (3.35 g, 96% yield) as a white solid. The material obtained was used directly without further purification. LC-MS calculated for Cl₄H₁₇BrFN₂O₃(M-C₄H₈+H)⁺: m/z=359.0. found 359.1.

### Intermediate 35. (2R,5S)-1-(1-(4-Bromo-2-fluorophenyl)-2-methylpropyl)-2,5-dimethylpiperazine Dihydrochloride

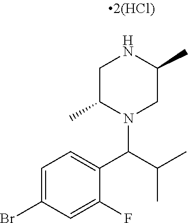 [SMILES: CC(C)C(c1ccc(Br)cc1F)N1C[C@H](C)NC[C@H]1C]

The title compound was prepared according to the procedures described in Intermediate 33, with tert-butyl (2S,5R)-4-(4-bromo-2-fluorobenzoyl)-2,5-dimethylpiperazine-1-carboxylate (Intermediate 34) replacing of tert-butyl (2S,5R)-4-(4-(difluoromethyl)-2-fluorobenzoyl)-2,5-dimethylpiperazine-1-carboxylate in Step 1. LC-MS calculated for C₁₆H₂₅BrFN₂ (M+H)⁺: m/z=343.1. found 343.1.

### Intermediate 36. tert-Butyl (2S,5R)-4-(4-chloro-2-fluorobenzoyl)-2,5-dimethylpiperazine-1-carboxylate

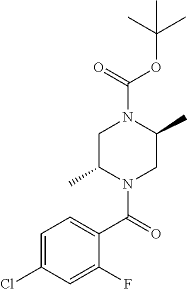 [SMILES: C[C@@H]1CN(C(=O)OC(C)(C)C)[C@@H](C)CN1C(=O)c1ccc(Cl)cc1F]

The title compound was prepared according to the procedures described in Intermediate 34, with 4-chloro-2-fluorobenzoyl chloride replacing 4-bromo-2-fluorobenzoyl chloride in Step 1. LC-MS calculated for C₁₄H₁₇ClFN₂O₃(M-C₄H₈+H)⁺: m/z=315.1. found 315.1.

### Intermediate 37. (2R,5S)-1-(1-(4-Chloro-2-fluorophenyl)-2-methylpropyl)-2,5-dimethylpiperazine Dihydrochloride

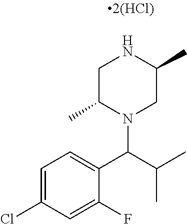 [SMILES: CC(C)C(c1ccc(Cl)cc1F)N1C[C@H](C)NC[C@H]1C]

The title compound was prepared according to the procedures described in Intermediate 33, with tert-butyl (2S,5R)-4-(4-chloro-2-fluorobenzoyl)-2,5-dimethylpiperazine-1-carboxylate (Intermediate 36) replacing of tert-butyl (2S,5R)-4-(4-(difluoromethyl)-2-fluorobenzoyl)-2,5-dimethylpiperazine-1-carboxylate in Step 1. LC-MS calculated for C₁₆H₂₅ClFN₂ (M+H)⁺: m/z=299.2. found 299.1.

### Intermediate 38. (2R,5S)-1-((4-Chloro-3-fluorophenyl)(3,3-difluorocyclobutyl)methyl)-2,5-dimethylpiperazine Hydrochloride

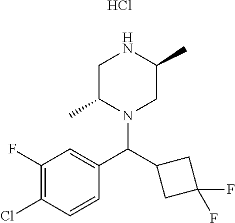 [SMILES: Cl ; C[C@@H]1CN[C@@H](C)CN1C(c1ccc(Cl)c(F)c1)C1CC(F)(F)C1]

The title compound was prepared according to the procedures described in Intermediate 3, with 1-chloro-2-fluoro-4-iodobenzene replacing 1-bromo-4-(trifluoromethyl)benzene in Step 1. LC-MS calculated for C₁₇H₂₃ClF₃N₂(M+H)⁺: m/z=347.2. found 347.1.

### Intermediate 39. (2R,5S)-1-((4-Chlorophenyl)(3,3-difluorocyclobutyl)methyl)-2,5-dimethylpiperazine Hydrochloride

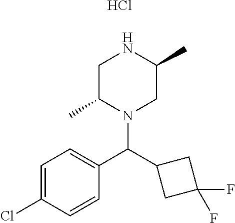 [SMILES: Cl ; C[C@@H]1CN[C@@H](C)CN1C(c1ccc(Cl)cc1)C1CC(F)(F)C1]

The title compound was prepared according to the procedures described in Intermediate 3, with 1-bromo-4-chlorobenzene replacing 1-bromo-4-(trifluoromethyl)benzene in Step 1. LC-MS calculated for C₁₇H₂₄ClF₂N₂(M+H)⁺: m/z=329.2. found 329.1.

### Intermediate 40. (2R,5S)-1-((4-Bromophenyl)(3,3-difluorocyclobutyl)methyl)-2,5-dimethylpiperazine Hydrochloride

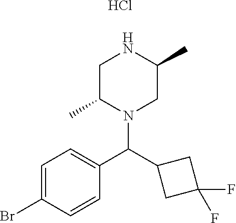 [SMILES: Cl ; C[C@@H]1CN[C@@H](C)CN1C(c1ccc(Br)cc1)C1CC(F)(F)C1]

The title compound was prepared according to the procedures described in Intermediate 3, with 1-bromo-4-iodobenzene replacing 1-bromo-4-(trifluoromethyl)benzene in Step 1. LC-MS calculated for Cl₇H₂₄BrF₂N₂(M+H)⁺. m/z=373.1. found 373.1.

### Intermediate 41. tert-Butyl (2S,5R)-5-ethyl-2-methylpiperazine-1-carboxylate

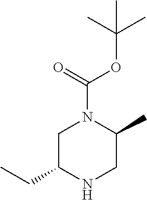 [SMILES: CC[C@@H]1CN(C(=O)OC(C)(C)C)[C@@H](C)CN1]

### Step 1: Methyl (R)-2-(benzylamino)butanoate

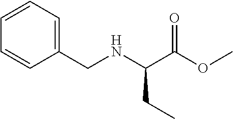

To a stirred solution of methyl (R)-2-aminobutanoate hydrochloride (30.0 g, 195 mmol, Combi-Blocks QA-7768) in CH₂Cl₂ (500 mL) was added benzaldehyde (20.7 g, 195 mmol) and the reaction mixture was stirred at rt for 6 h. The reaction mixture was cooled to 0° C. in an ice-bath before sodium triacetoxyborohydride (20.7 g, 98 mmol) was added portionwise over 20 min. The ice-bath was removed and the reaction mixture was stirred at ambient temperature overnight. The mixture was transferred to a separatory funnel and extracted with 1 M aqueous HCl (3×300 mL). The combined aqueous layers were made basic with solid KOH (pH>12) and extracted with EtOAc (3×300 mL). The combined organic layers were washed with saturated aqueous NaCl, dried over MgSO₄, and the filtrate was concentrated to afford the desired product (28.3 g, 70% yield) as a colorless oil. The crude material obtained was used directly without further purification. LC-MS calculated for C₁₂H₁₈NO₂ (M+H)⁺: m/z=208.1. found 208.2.

### Step 2: Methyl (R)-2-((S)—N-benzyl-2-((tert-butoxycarbonyl)amino)propanamido)butanoate

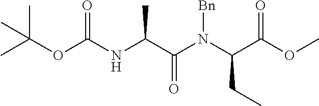 [SMILES: CC[C@H](C(=O)OC)N(Cc1ccccc1)C(=O)[C@H](C)NC(=O)OC(C)(C)C]

To a mixture of methyl (R)-2-(benzylamino)butanoate (18.4 g, 89 mmol) and (tert-butoxycarbonyl)-L-alanine (21.8 g, 115 mmol, Combi-Blocks QA-6543) in N,N-dimethylformamide (100 mL) was added HATU (50.6 g, 133 mmol, Oakwood 023926) followed by N-ethyl-N-isopropylpropan-2-amine (41.9 mL, 240 mmol) and the reaction mixture was stirred at rt overnight. The mixture was diluted with Et₂O (600 mL) and washed with water (200 mL). After phase separation the organic layer was removed and the aqueous layer was extracted with Et₂O (2×200 mL). The combined organic layers were dried over MgSO₄, concentrated, and the crude residue was purified by flash column chromatography (SiO₂, EtOAc/hexanes) to afford the desired product (30 g, 89% yield). LC-MS calculated for C₂₀H₃₁N₂O₅ (M+H)⁺: m/z=379.2. found 379.3.

### Step 3: (3S,6R)-1-Benzyl-6-ethyl-3-methylpiperazine-2,5-dione

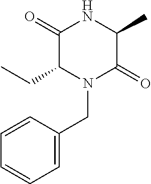 [SMILES: CC[C@@H]1C(=O)N[C@@H](C)C(=O)N1Cc1ccccc1]

To a mixture of methyl (R)-2-((S)—N-benzyl-2-((tert-butoxycarbonyl)amino)propanamido)butanoate (30 g, 79 mmol) in CH₂Cl₂ (200 mL) was added trifluoroacetic acid (50 mL, 649 mmol) and the reaction mixture was stirred at rt overnight. The reaction mixture was concentrated in vacuo. To the crude residue was added MeOH (200 mL) and the reaction mixture was sealed and stirred at 70° C. overnight. After cooling to rt, the reaction mixture was concentrated in vacuo to afford the desired product (27 g). The crude material obtained was used directly without further purification. LC-MS calculated for C₁₄H₁₉N₂O₂ (M+H)⁺: m/z=247.1. found 247.2.

### Step 4: (2R,5S)-1-Benzyl-2-ethyl-5-methylpiperazine

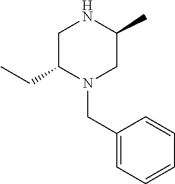 [SMILES: CC[C@@H]1CN[C@@H](C)CN1Cc1ccccc1]

A mixture of (3S,6R)-1-benzyl-6-ethyl-3-methylpiperazine-2,5-dione (Step 3) in THF (200 mL) was cooled to 0° C. in an ice-bath before borane tetrahydrofuran complex (1 M in THF, 375 mL, 375 mmol, Aldrich 176192) was added slowly. The ice-bath was removed and the reaction mixture was stirred at 70° C. for 20 h. After cooling to rt, the reaction mixture was quenched via the slow addition of MeOH (100 mL) followed by 1 M aqueous HCl (112 mL, 112 mmol). The mixture was stirred at 70° C. for an additional 2 h. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄, and concentrated. The crude material obtained was used directly without further purification. LC-MS calculated for C₁₄H₂₃N₂(M+H)⁺: m/z=219.2. found 219.1.

### Step 5: tert-Butyl (2S,5R)-4-benzyl-5-ethyl-2-methylpiperazine-1-carboxylate

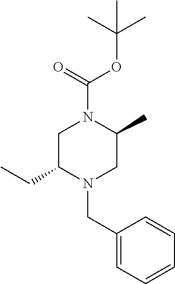 [SMILES: CC[C@@H]1CN(C(=O)OC(C)(C)C)[C@@H](C)CN1Cc1ccccc1]

To a mixture of (2R,5S)-1-benzyl-2-ethyl-5-methylpiperazine (Step 4) in CH₂C12 (150 mL) was added triethylamine (31.3 mL, 225 mmol) and di-tert-butyl dicarbonate (26.1 mL, 112 mmol) and the reaction mixture was stirred at rt overnight. The mixture was diluted with CH₂Cl₂ and washed with water (150 mL) and saturated aqueous NaCl. The organic layer was dried over MgSO₄, concentrated, and the crude residue was purified by flash column chromatography (SiO₂, EtOAc/hexanes) to afford the desired product (22.2 g) as an off-white solid. LC-MS calculated for C₁₉H₃₁N₂O₂ (M+H)⁺: m/z=319.2. found 319.3.

### Step 6: tert-Butyl (2S,5R)-5-ethyl-2-methylpiperazine-1-carboxylate

To a mixture of tert-butyl (2S,5R)-4-benzyl-5-ethyl-2-methylpiperazine-1-carboxylate (22.2 g, 69.7 mmol) in MeOH (170 mL) was added palladium on carbon (10 wt %, 3.2 g, 3 mmol) and the reaction mixture was shaken in a Parr shaker under 50 psi of H₂ (g) for 20 h. The mixture was filtered over a pad of Celite®, and the filter cake was washed with MeOH (170 mL). The filtrate was concentrated and dried under vacuum to afford the desired product (12.5 g, 78% yield). The material obtained was used directly without further purification. LC-MS calculated for C₁₂H₂₅N₂O₂ (M+H)⁺: m/z=229.2. found 229.3.

### Intermediate 42. tert-Butyl (2S,5R)-4-(3,3-difluorocyclobutane-1-carbonyl)-5-ethyl-2-methylpiperazine-1-carboxylate

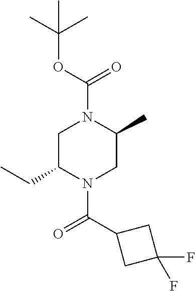 [SMILES: CC[C@@H]1CN(C(=O)OC(C)(C)C)[C@@H](C)CN1C(=O)C1CC(F)(F)C1]

The title compound was prepared according to the procedures described in Intermediate 2, with tert-butyl (2S,5R)-5-ethyl-2-methylpiperazine-1-carboxylate (Intermediate 41) replacing tert-butyl (2S,5R)-2,5-dimethylpiperazine-1-carboxylate. LC-MS calculated for C₁₃H₂₁F₂N₂O₃(M-C₄H₈+H)⁺: m/z=291.2. found 291.1.

### Intermediate 43. (2R,5S)-1-((4-Chlorophenyl)(3,3-difluorocyclobutyl)methyl)-2-ethyl-5-methylpiperazine Hydrochloride

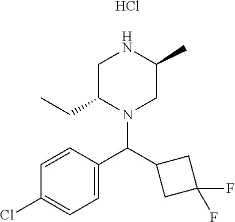 [SMILES: Cl ; CC[C@@H]1CN[C@@H](C)CN1C(c1ccc(Cl)cc1)C1CC(F)(F)C1]

### Step 1: (4-Chlorophenyl)magnesium Chloride Lithium Chloride (1.1 M in THF)

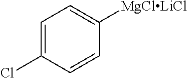

A 1.3 M solution of isopropylmagnesium chloride lithium chloride complex in THF (5.78 mL, 7.52 mmol, Aldrich 656984) was cooled to −78° C. before 1-bromo-4-chlorobenzene (0.96 mL, 8.3 mmol) was added dropwise and the reaction mixture was stirred at −78° C. for 5 min. The reaction mixture was warmed to rt and stirred for an additional 4 h. The mixture obtained was used directly in the next step.

### Step 2: tert-Butyl (2S,5R)-4-((4-chlorophenyl)(3,3-difluorocyclobutyl)methyl)-5-ethyl-2-methylpiperazine-1-carboxylate

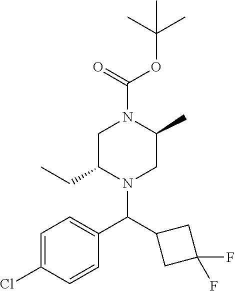 [SMILES: CC[C@@H]1CN(C(=O)OC(C)(C)C)[C@@H](C)CN1C(c1ccc(Cl)cc1)C1CC(F)(F)C1]

A mixture of tert-butyl (2S,5R)-4-(3,3-difluorocyclobutane-1-carbonyl)-5-ethyl-2-methylpiperazine-1-carboxylate (Intermediate 42, 0.800 g, 2.31 mmol) and chlorocarbonylbis(triphenylphosphine)iridium(I) (180 mg, 0.231 mmol, Strem 77-0300) in CH₂Cl₂ (5 mL) was treated with 1,1,3,3-tetramethyldisiloxane (816 μL, 4.62 mmol, Aldrich 235733) and stirred at rt for 25 min. The reaction was cooled to −78° C. and stirred for 5 min before (4-chlorophenyl)magnesium chloride lithium chloride (Step 1, 2.89 mL, 1.1 M in THF, 3.2 mmol) was added dropwise and the reaction mixture was stirred for an additional 5 min. The reaction mixture was warmed to 0° C. and stirred for 3 h. The mixture was quenched with saturated aqueous NH₄Cl. After warming to rt, the organic layer was removed and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₂₃H₃₄ClF₂N₂O₂ (M+H)⁺: m/z=443.2. found 443.3.

### Step 3: (2R,5S)-1-((4-Chlorophenyl)(3,3-difluorocyclobutyl)methyl)-2-ethyl-5-methylpiperazine Hydrochloride

A mixture of tert-butyl (2S,5R)-4-((4-chlorophenyl)(3,3-difluorocyclobutyl)methyl)-5-ethyl-2-methylpiperazine-1-carboxylate (Step 2) in THF (15 mL) was treated with HCl (4 M in 1,4-dioxane, 5 mL, 20 mmol, Oakwood 094030) and stirred at 60° C. for 30 min. The mixture was then diluted with diethyl ether and the precipitate was collected by filtration and washed with diethyl ether to afford the desired product as mixture of diastereomers in the form of a white solid. LC-MS calculated for C₁₈H₂₆ClF₂N₂(M+H)⁺: m/z=343.2. found 343.2.

### Intermediate 44. (2R,5S)-1-((3,3-Difluorocyclobutyl)(4-(trifluoromethyl)phenyl)methyl)-2-ethyl-5-methylpiperazine Hydrochloride

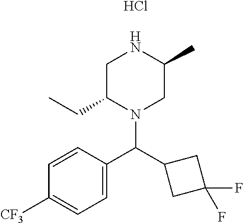 [SMILES: Cl ; CC[C@@H]1CN[C@@H](C)CN1C(c1ccc(C(F)(F)F)cc1)C1CC(F)(F)C1]

The title compound was prepared according to the procedures described in Intermediate 43, with 1-bromo-4-(trifluoromethyl)benzene replacing 1-bromo-4-chlorobenzene in Step 1. LC-MS calculated for C₁₉H₂₆F₅N₂ (M+H)⁺. m/z=377.2. found 377.2.

### Intermediate 45. 4-((2S,5R)-2,5-Dimethylpiperazin-1-yl)-2-methyl-1-(((S)-tetrahydrofuran-2-yl)methyl)-1H-[1,2,4]triazolo[3,4-b]purine Hydrochloride

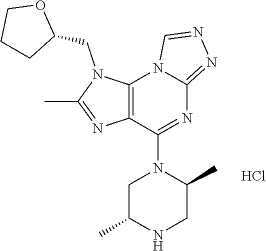 [SMILES: Cc1nc2c(N3C[C@@H](C)NC[C@@H]3C)nc3nncn3c2n1C[C@@H]1CCCO1 ; Cl]

### Step 1. tert-Butyl (2R,5S)-4-(2-chloro-8-methyl-9-(((S)-tetrahydrofuran-2-yl)methyl)-9H-purin-6-yl)-2,5-dimethylpiperazine-1-carboxylate

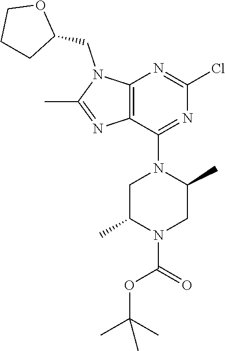 [SMILES: Cc1nc2c(N3C[C@@H](C)N(C(=O)OC(C)(C)C)C[C@@H]3C)nc(Cl)nc2n1C[C@@H]1CCCO1]

To a mixture of (S)-2,6-dichloro-8-methyl-9-((tetrahydrofuran-2-yl)methyl)-9H-purine (Intermediate 5, 1.02 g, 5.0 mmol) and tert-butyl (2R,5S)-2,5-dimethylpiperazine-1-carboxylate (1.07 g, 5.0 mmol) in MeCN (10.0 mL) was added potassium carbonate (1.38 g, 10.0 mmol) and the reaction mixture was stirred at 90° C. overnight. After cooling to rt, the reaction mixture was filtered over Celite and the filtrate was concentrated in vacuo. The crude residue was purified directly by flash column chromatography (24 g SiO₂, EtOAc/hexanes) to afford the desired product as a light yellow solid. LC-MS calculated for C₂₂H₃₄ClN₆O₃(M+H)⁺: m/z=465.2. found 465.3.

### Step 2. tert-Butyl (2R,5S)-4-(2-hydrazineyl-8-methyl-9-(((S)-tetrahydrofuran-2-yl)methyl)-9H-purin-6-yl)-2,5-dimethylpiperazine-1-carboxylate

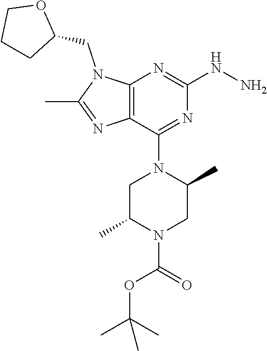 [SMILES: Cc1nc2c(N3C[C@@H](C)N(C(=O)OC(C)(C)C)C[C@@H]3C)nc(NN)nc2n1C[C@@H]1CCCO1]

To a mixture of tert-butyl (2R,5S)-4-(2-chloro-8-methyl-9-(((S)-tetrahydrofuran-2-yl)methyl)-9H-purin-6-yl)-2,5-dimethylpiperazine-1-carboxylate (465 mg, 1.0 mmol), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (85 mg, 0.10 mmol), and cesium carbonate (1.63 g, 5.0 mmol) was added a 1 molar solution of hydrazine in THF (5.0 mL, 5.0 mmol) and the mixture was stirred at 90° C. for 30 min. After cooling to rt, the reaction mixture was filtered through a pad of MgSO₄ in a SiliaPrep SPE thiol cartridge (SiliCycle SPE-R51030B-06P). The filtrate was concentrated, and the crude material obtained was used directly without further purification. LC-MS calculated for C₂₂H₃₇N₈O₃ (M+H)⁺: m/z=461.3. found 461.3.

### Step 3. tert-Butyl (2R,5S)-2,5-dimethyl-4-(2-methyl-1-(((S)-tetrahydrofuran-2-yl)methyl)-1H-[1,2,4]triazolo[3,4-b]purin-4-yl)piperazine-1-carboxylate

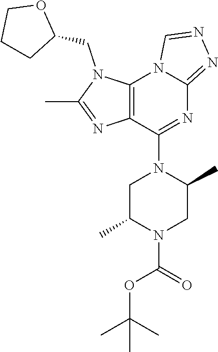 [SMILES: Cc1nc2c(N3C[C@@H](C)N(C(=O)OC(C)(C)C)C[C@@H]3C)nc3nncn3c2n1C[C@@H]1CCCO1]

A mixture of tert-butyl (2R,5S)-4-(2-hydrazineyl-8-methyl-9-(((S)-tetrahydrofuran-2-yl)methyl)-9H-purin-6-yl)-2,5-dimethylpiperazine-1-carboxylate (Step 2), triethyl orthoformate (2.0 mL, 12.0 mmol), and AcOH (0.4 mL, 7.0 mmol) was stirred at 90° C. overnight. After cooling to rt, the reaction mixture was diluted with EtOAc and water. The layers were separated and the aqueous layer was extracted with EtOAc. The combined organic layers were washed with brine, dried over MgSO₄, concentrated in vacuo, and purified by flash column chromatography (12 g SiO₂, MeOH/CH₂Cl₂) to give the desired product as a yellow solid. LC-MS calculated for C₂₃H₃₅N₈O₃ (M+H)⁺: m/z=471.3. found 471.3.

### Step 4. 4-((2S,5R)-2,5-Dimethylpiperazin-1-yl)-2-methyl-1-(((S)-tetrahydrofuran-2-yl)methyl)-1H-[1,2,4]triazolo[3,4-b]purine Hydrochloride

To a mixture of tert-butyl (2R,5S)-2,5-dimethyl-4-(2-methyl-1-(((S)-tetrahydrofuran-2-yl)methyl)-1H-[1,2,4]triazolo[3,4-b]purin-4-yl)piperazine-1-carboxylate (Step 3) in CH₂Cl₂ (1.0 mL) was added a 4 molar solution of HCl in 1,4-dioxane (0.50 mL, 2.0 mmol), and the reaction mixture was allowed to stir at rt for 4 h. The reaction mixture was concentrated in vacuo to give desired product as a light yellow solid. LC-MS calculated for C₁₈H₂₇N₈O (M+H)⁺: m/z=371.2. found 371.3.

### Intermediate 46. 4-((2S,5R)-2,5-Dimethylpiperazin-1-yl)-1-(((S)-tetrahydrofuran-2-yl)methyl)-1H-[1,2,4]triazolo[3,4-b]purine Hydrochloride

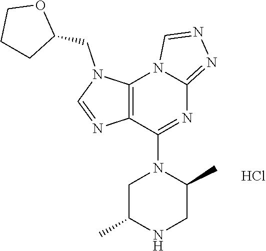 [SMILES: C[C@@H]1CN(c2nc3nncn3c3c2ncn3C[C@@H]2CCCO2)[C@@H](C)CN1 ; Cl]

The title compound was prepared according to the procedures described in Intermediate 45, with (S)-2,6-dichloro-9-((tetrahydrofuran-2-yl)methyl)-9H-purine (Intermediate 1) replacing (S)-2,6-dichloro-8-methyl-9-((tetrahydrofuran-2-yl)methyl)-9H-purine in Step 1. LC-MS calculated for C₁₇H₂₅N₈O (M+H)⁺. m/z=357.2. found 357.2.

### Intermediate 47. 4-((2S,5R)-2,5-Dimethylpiperazin-1-yl)-2-methyl-1-(((S)-tetrahydrofuran-2-yl)methyl)-1H-imidazo[4,5-e][1,2,4]triazolo[4,3-a]pyridine hydrochloride

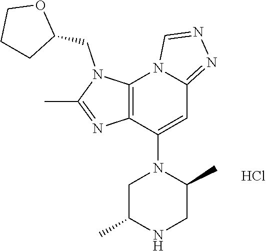 [SMILES: Cc1nc2c(N3C[C@@H](C)NC[C@@H]3C)cc3nncn3c2n1C[C@@H]1CCCO1 ; Cl]

### Step 1. tert-Butyl (2R,5S)-4-(2-amino-6-chloro-3-nitropyridin-4-yl)-2,5-dimethylpiperazine-1-carboxylate

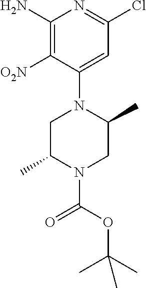 [SMILES: C[C@@H]1CN(c2cc(Cl)nc(N)c2[N+](=O)[O-])[C@@H](C)CN1C(=O)OC(C)(C)C]

A mixture of tert-butyl (2R,5S)-2,5-dimethylpiperazine-1-carboxylate (1.07 g, 5.0 mmol) (214 mg, 1.0 mmol) and 4,6-dichloro-3-nitropyridin-2-amine (208 mg, 1.0 mmol, ChemScene CS-0094679) in MeCN (5.0 mL) was cooled to 0° C. in an ice-bath before N,N-diisopropylethylamine (0.35 mL, 2.0 mmol) was added and the reaction mixture was stirred at 90° C. overnight. The mixture was diluted with saturated aqueous NaHCO₃ and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₁₆H₂₅ClN₅O₄(M+H)⁺: m/z=386.2. found 386.2.

### Step 2. tert-Butyl (2R,5S)-4-(2,3-diamino-6-chloropyridin-4-yl)-2,5-dimethylpiperazine-1-carboxylate

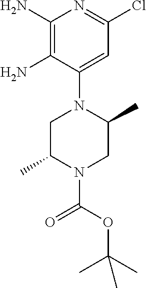 [SMILES: C[C@@H]1CN(c2cc(Cl)nc(N)c2N)[C@@H](C)CN1C(=O)OC(C)(C)C]

A mixture of tert-butyl (2R,5S)-4-(2-amino-6-chloro-3-nitropyridin-4-yl)-2,5-dimethylpiperazine-1-carboxylate (Step 1) in DMF (2.0 mL) was cooled to 0° C. in an ice-bath before hypodiboric acid (0.269 g, 3.0 mmol) was added, followed by dropwise addition of a solution of 4,4′-dipyridyl (1.6 mg, 10.0 μmol) in DMF (0.5 mL). The mixture was stirred at 0° C. for 5 min, at which point the mixture was diluted with water and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₁₆H₂₇ClN₅O₂(M+H)⁺: m/z=356.2. found 356.2.

### Step 3. tert-Butyl (2R,5S)-4-(5-chloro-2-methyl-3H-imidazo[4,5-b]pyridin-7-yl)-2,5-dimethylpiperazine-1-carboxylate

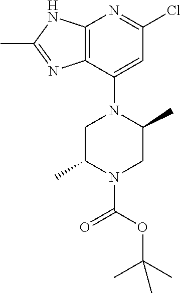

A mixture of tert-butyl (2R,5S)-4-(2,3-diamino-6-chloropyridin-4-yl)-2,5-dimethylpiperazine-1-carboxylate (Step 2) and acetic acid (0.11 mL, 1.92 mmol) in triethyl orthoacetate (1.0 mL) was stirred at 120° C. for 4 h. The mixture was diluted with saturated aqueous NaHCO₃ and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₁₈H₂₇ClN₅O₂(M+H)⁺: m/z=380.2. found 380.2.

### Step 4. tert-Butyl (2R,5S)-4-(5-chloro-2-methyl-3-(((S)-tetrahydrofuran-2-yl)methyl)-3H-imidazo[4,5-b]pyridin-7-yl)-2,5-dimethylpiperazine-1-carboxylate

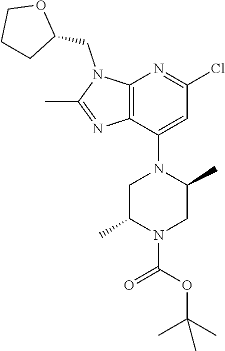 [SMILES: Cc1nc2c(N3C[C@@H](C)N(C(=O)OC(C)(C)C)C[C@@H]3C)cc(Cl)nc2n1C[C@@H]1CCCO1]

A mixture of tert-butyl (2R,5S)-4-(5-chloro-2-methyl-3H-imidazo[4,5-b]pyridin-7-yl)-2,5-dimethylpiperazine-1-carboxylate (Step 3) in MeCN (1.0 mL) was added cesium carbonate (489 mg, 1.5 mmol) and (S)-(tetrahydrofuran-2-yl)methyl methanesulfonate (Intermediate 50, 108 mg, 0.6 mmol) and the reaction mixture was stirred at 90° C. overnight. After cooling to rt, the reaction mixture was filtered through a pad of Celite and concentrated in vacuo. The crude residue was purified by flash column chromatography (12 g SiO₂, EtOAc/hexanes) to afford the desired product as a white solid. LC-MS calculated for C₂₃H₃₅ClN₅O₃(M+H)⁺. m/z=464.2. found 464.2.

### Step 5. tert-Butyl (2R,5S)-4-(5-hydrazineyl-2-methyl-3-(((S)-tetrahydrofuran-2-yl)methyl)-3H-imidazo[4,5-b]pyridin-7-yl)-2,5-dimethylpiperazine-1-carboxylate

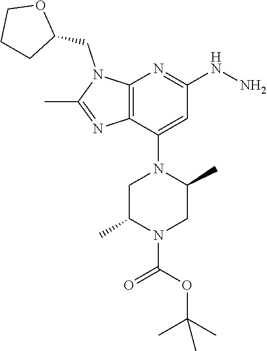 [SMILES: Cc1nc2c(N3C[C@@H](C)N(C(=O)OC(C)(C)C)C[C@@H]3C)cc(NN)nc2n1C[C@@H]1CCCO1]

To a mixture of tert-butyl (2R,5S)-4-(5-chloro-2-methyl-3-(((S)-tetrahydrofuran-2-yl)methyl)-3H-imidazo[4,5-b]pyridin-7-yl)-2,5-dimethylpiperazine-1-carboxylate (232 mg, 0.5 mmol), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (43 mg, 0.05 mmol), and cesium carbonate (815 mg, 2.5 mmol) was added a 1 molar solution of hydrazine in THF (0.5 mL, 0.5 mmol) and the mixture was stirred at 90° C. for 30 min. After cooling to rt, the reaction mixture was filtered through a pad of MgSO₄ in a SiliaPrep SPE thiol cartridge (SiliCycle SPE-R51030B-06P). The filtrate was concentrated, and the crude material obtained was used directly without further purification. LC-MS calculated for C₂₃H₃₈N₇O₃ (M+H)⁺: m/z=460.3. found 460.4.

### Step 6. tert-Butyl (2R,5S)-2,5-dimethyl-4-(2-methyl-1-(((S)-tetrahydrofuran-2-yl)methyl)-1H-imidazo[4,5-e][1,2,4]triazolo[4,3-a]pyridin-4-yl)piperazine-1-carboxylate

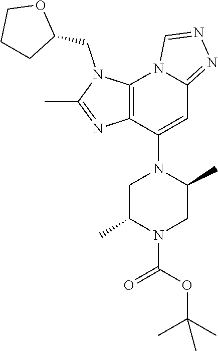 [SMILES: Cc1nc2c(N3C[C@@H](C)N(C(=O)OC(C)(C)C)C[C@@H]3C)cc3nncn3c2n1C[C@@H]1CCCO1]

A mixture of tert-butyl (2R,5S)-4-(5-hydrazineyl-2-methyl-3-(((S)-tetrahydrofuran-2-yl)methyl)-3H-imidazo[4,5-b]pyridin-7-yl)-2, 5-dimethylpiperazine-1-carboxylate (Step 5), triethyl orthoformate (1.0 mL, 6.0 mmol), and AcOH (0.2 mL, 3.5 mmol) was stirred at 90° C. overnight. After cooling to rt, the reaction mixture was diluted with EtOAc and water. The layers were separated and the aqueous layer was extracted with EtOAc. The combined organic layers were washed with brine, dried over MgSO₄, concentrated in vacuo, and purified by flash column chromatography (12 g SiO₂, MeOH/CH₂Cl₂) to give the desired product as a yellow solid. LC-MS calculated for C₂₄H₃₆N₇O₃ (M+H)⁺: m/z=470.3. found 470.3.

### Step 7. 4-((2S,5R)-2,5-Dimethylpiperazin-1-yl)-2-methyl-1-(((S)-tetrahydrofuran-2-yl)methyl)-1H-imidazo[4,5-e][1,2,4]triazolo[4,3-a]pyridine Hydrochloride

To a mixture of tert-butyl (2R,5S)-2,5-dimethyl-4-(2-methyl-1-(((S)-tetrahydrofuran-2-yl)methyl)-1H-imidazo[4,5-e][1,2,4]triazolo[4,3-a]pyridin-4-yl)piperazine-1-carboxylate (Step 6) in CH₂Cl₂ (1.0 mL) was added a 4 molar solution of HCl in 1,4-dioxane (0.5 mL, 2 mmol), and the reaction mixture was allowed to stir at rt for 4 h. The reaction mixture was concentrated in vacuo to give desired product as a light yellow solid. LC-MS calculated for C₁₉H₂₈N₇O (M+H)⁺: m/z=370.2. found 370.3.

### Intermediate 48. 4-((2S,5R)-2,5-Dimethylpiperazin-1-yl)-1-(((S)-tetrahydrofuran-2-yl)methyl)-1H-imidazo[4,5-e][1,2,4]triazolo[4,3-a]pyridine Hydrochloride

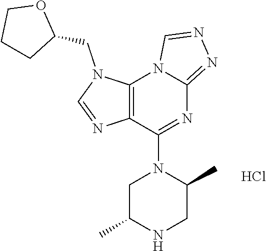 [SMILES: C[C@@H]1CN(c2nc3nncn3c3c2ncn3C[C@@H]2CCCO2)[C@@H](C)CN1 ; Cl]

The title compound was prepared according to the procedures described in Intermediate 47, with triethyl orthoformate replacing triethyl orthoacetate in Step 3. LC-MS calculated for C₁₈H₂₆N₇O (M+H)⁺: m/z=356.2. found 356.2.

### Intermediate 49. (2R,5S)-1-(Bis(4-fluorophenyl)methyl)-2,5-dimethylpiperazine Hydrochloride

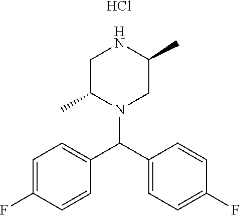 [SMILES: Cl ; C[C@@H]1CN[C@@H](C)CN1C(c1ccc(F)cc1)c1ccc(F)cc1]

### Step 1. tert-Butyl (2S,5R)-4-(bis(4-fluorophenyl)methyl)-2,5-dimethylpiperazine-1-carboxylate

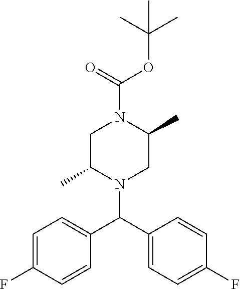 [SMILES: C[C@@H]1CN(C(=O)OC(C)(C)C)[C@@H](C)CN1C(c1ccc(F)cc1)c1ccc(F)cc1]

A mixture of tert-butyl (2S,5R)-2,5-dimethylpiperazine-1-carboxylate (15.0 g, 70.0 mmol, Combi-Blocks OR-8588), 4,4′-(chloromethylene)bis(fluorobenzene) (19.2 g, 80.0 mmol, Combi-Blocks QA-4728) and N-ethyl-N-isopropylpropan-2-amine (37 mL, 210 mmol) in CH₃CN (175 mL) was stirred at 85° C. overnight. After cooling to rt, the reaction mixture was concentrated in vacuo and the residue was dissolved in EtOAc and washed with water and brine. The organic phase was dried over MgSO₄, filtered, and concentrated and the crude residue was purified using flash column chromatography (330 g SiO₂, EtOAc/hexanes) to afford tert-butyl (2S,5R)-4-(bis(4-fluorophenyl)methyl)-2,5-dimethylpiperazine-1-carboxylate (26.0 g, 89% yield) as a light yellow waxy solid. LC-MS calculated for C₂₄H₃₁F₂N₂O₂(M+H)⁺: m/z=417.2. found 417.1.

### Step 2. (2R,5S)-1-(Bis(4-fluorophenyl)methyl)-2,5-dimethylpiperazine Hydrochloride

To a mixture of tert-butyl (2S,5R)-4-(bis(4-fluorophenyl)methyl)-2,5-dimethylpiperazine-1-carboxylate (1.86 g, 4.50 mmol) in THF (25 mL) was added a 4 molar solution of HCl in 1,4-dioxane (6.25 mL, 25.0 mmol) and the reaction mixture was purged with N₂ and stirred at 80° C. for 4 h. After cooling to rt, the reaction mixture was diluted with Et₂O (25 mL) and hexanes (50 mL) and slurried for 30 mins. The solid precipitate was collected via filtration, washed with Et₂O and hexanes, and dried under vacuum to afford the desired product (1.34 g, 85% yield) as a white solid. LC-MS calculated for C₁₉H₂₃F₂N₂ (M+H)⁺: m/z=317.2. found 317.2.

### Intermediate 50. (S)-(Tetrahydrofuran-2-yl)methyl Methanesulfonate

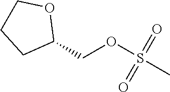

A mixture of (S)-(tetrahydrofuran-2-yl)methanol (2.00 g, 19.6 mmol, BLD Pharmatech BD48351) and N-ethyl-N-isopropylpropan-2-amine (5.12 mL, 29.4 mmol) in CH₂Cl₂ (15 mL) was purged with N₂ and cooled to 0° C. before methanesulfonyl chloride (1.97 mL, 25.5 mmol) was added dropwise. The reaction mixture was allowed to warm to rt and stirred for 30 mins. The mixture was quenched with saturated aqueous NaHCO₃, the organic layer was removed, and the aqueous layer was extracted with CH₂Cl₂. The combined organic layers were dried over MgSO₄, filtered, and concentrated under reduced pressure to afford the desired product (3.39 g, 96% yield) as a light orange oil that was used directly without further purification. ¹H NMR (400 MHz, CDCl₃) δ 4.28-4.20 (m, 1H), 4.20-4.13 (m, 2H), 3.88 (dt, J=8.4, 6.6 Hz, 1H), 3.80 (dt, J=8.2, 6.6 Hz, 1H), 3.05 (s, 3H), 2.08-1.97 (m, 1H), 1.97-1.87 (m, 2H), 1.73-1.63 (m, 1H).

### Intermediate 51. (2R,5S)-1-(Bis(4-chlorophenyl)methyl)-2,5-dimethylpiperazine Hydrochloride

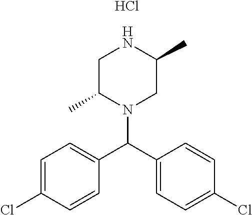 [SMILES: Cl ; C[C@@H]1CN[C@@H](C)CN1C(c1ccc(Cl)cc1)c1ccc(Cl)cc1]

### Step 1: tert-Butyl (2S,5R)-4-(bis(4-chlorophenyl)methyl)-2,5-dimethylpiperazine-1-carboxylate

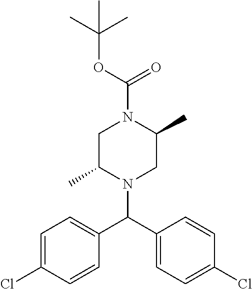 [SMILES: C[C@@H]1CN(C(=O)OC(C)(C)C)[C@@H](C)CN1C(c1ccc(Cl)cc1)c1ccc(Cl)cc1]

In a 20 mL microwave vial with a stir bar, a mixture of tert-butyl (2S,5R)-2,5-dimethylpiperazine-1-carboxylate (1.11 g, 5.19 mmol, Combi-Blocks OR-8588), 4,4′-(chloromethylene)bis(chlorobenzene) (1.41 g, 5.19 mmol, A2B Chem AC49945), and N,N-diisopropylethylamine (1.81 mL, 10.4 mmol) in MeCN (13 mL) was irradiated at 115° C. in a microwave reactor for 4 h. A second reaction was set up in parallel in a separate vessel, and after cooling to rt the two reaction mixtures were combined and concentrated in vacuo. The crude residue was diluted with EtOAc and water and the layers were separated. The organic layer was removed, and the aqueous layer was extracted with EtOAc. The combined organic layers were washed with brine, dried over MgSO₄, and concentrated in vacuo. The crude residue was purified by flash column chromatography (40 g SiO₂, EtOAc/hexanes) to give the title compound as a white solid. LC-MS calculated for C₂₄H₃₁Cl₂N₂O₂ (M+H)⁺: m/z=449.2. found 449.2.

### Step 2: (2R,5S)-1-(Bis(4-chlorophenyl)methyl)-2,5-dimethylpiperazine Hydrochloride

A mixture of tert-butyl (2S,5R)-4-(bis(4-chlorophenyl)methyl)-2,5-dimethylpiperazine-1-carboxylate (Step 1) in THF (26 mL) was treated with HCl (4 M in 1,4-dioxane, 26 mL, 104 mmol) and the reaction mixture was stirred at 60° C. for 1 h. After cooling to rt, the mixture was diluted with diethyl ether (100 mL). The solid precipitate that formed was collected by filtration, washed with diethyl ether, and dried under vacuum to give the title compound (2.44 g, 61% yield over two steps) as a white solid. LC-MS calculated for C₁₉H₂₃Cl₂N₂(M+H)⁺: m/z=349.1. found 349.2.

### Intermediate 52. 5-Chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine

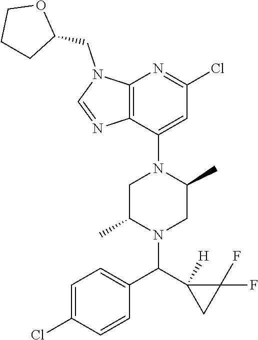 [SMILES: C[C@@H]1CN(c2cc(Cl)nc3c2ncn3C[C@@H]2CCCO2)[C@@H](C)CN1C(c1ccc(Cl)cc1)[C@@H]1CC1(F)F]

### Step 1. 6-Chloro-4-((2S,5R)-4-((4-chlorophenyl)((S)-2,2-difluorocyclopropyl)methyl)-2,5-dimethylpiperazin-1-yl)-3-nitropyridin-2-amine

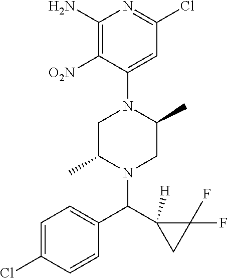 [SMILES: C[C@@H]1CN(c2cc(Cl)nc(N)c2[N+](=O)[O-])[C@@H](C)CN1C(c1ccc(Cl)cc1)[C@@H]1CC1(F)F]

A mixture of (2R,5S)-1-((4-chlorophenyl)((S)-2,2-difluorocyclopropyl)methyl)-2,5-dimethylpiperazine hydrochloride (Intermediate 16, 0.105 g, 0.300 mmol), 4,6-dichloro-3-nitropyridin-2-amine (62.0 mg, 0.300 mmol PharmaBlock PBT0266), and N,N-diisopropylethylamine (0.157 mL, 0.900 mmol) in MeCN (1 mL) was stirred at 90° C. overnight. After cooling to room temperature, the mixture was concentrated in vacuo and used without further purification. LC-MS calculated for C₂₁H₂₄Cl₂F₂N₅O₂(M+H)⁺: m/z=486.1. found 486.1.

### Step 2. 6-Chloro-4-((2S,5R)-4-((4-chlorophenyl)((S)-2,2-difluorocyclopropyl)methyl)-2,5-dimethylpiperazin-1-yl)pyridine-2,3-diamine

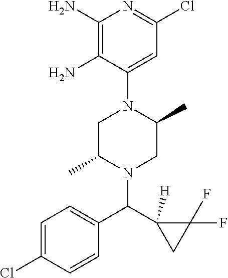 [SMILES: C[C@@H]1CN(c2cc(Cl)nc(N)c2N)[C@@H](C)CN1C(c1ccc(Cl)cc1)[C@@H]1CC1(F)F]

A mixture of 6-chloro-4-((2S,5R)-4-((4-chlorophenyl)((S)-2,2-difluorocyclopropyl)methyl)-2,5-dimethylpiperazin-1-yl)-3-nitropyridin-2-amine (Step 1), 4,4′-dipyridyl (4.7 mg, 0.030 mmol, Aldrich 289426), and tetrahydroxydiboron (81 mg, 0.90 mmol, BLD Pharmatech BD288251) in DMF (0.6 mL) was stirred at room temperature for 10 minutes. The mixture was diluted with CH₂Cl₂ and water and filtered through a pad of Celite®. The layers were separated, and the aqueous layer was extracted with CH₂Cl₂. The combined organic layers were dried over MgSO₄ and concentrated in vacuo. The crude product was used without further purification. LC-MS calculated for C₂₁H₂₆Cl₂F₂N₅ (M+H)⁺. m/z=456.2. found 456.1.

### Step 3. 5-Chloro-7-((2S,5R)-4-((4-chlorophenyl)((S)-2,2-difluorocyclopropyl)methyl)-2,5-dimethylpiperazin-1-yl)-3H-imidazo[4,5-b]pyridine

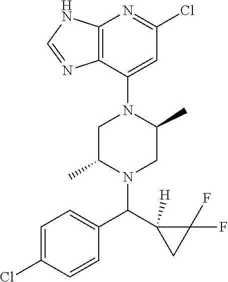 [SMILES: C[C@@H]1CN(c2cc(Cl)nc3[nH]cnc23)[C@@H](C)CN1C(c1ccc(Cl)cc1)[C@@H]1CC1(F)F]

A mixture of 6-chloro-4-((2S,5R)-4-((4-chlorophenyl)((S)-2,2-difluorocyclopropyl)methyl)-2,5-dimethylpiperazin-1-yl)pyridine-2,3-diamine (Step 2), triethyl orthoformate (0.125 mL, 0.750 mmol), and acetic acid (0.429 mL, 7.50 mmol) was stirred at 95° C. for 30 minutes. The mixture was cooled to room temperature, diluted with CH₂Cl₂, and quenched with saturated aq. NaHCO₃. The layers were separated and the aqueous layer was extracted with CH₂Cl₂. The combined organic layers were dried over MgSO₄ and concentrated in vacuo. The crude product was used without further purification. LC-MS calculated for C₂₂H₂₄Cl₂F₂N₅ (M+H)⁺: m/z=466.1. found 466.2.

### Step 4: 5-Chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine

A mixture of 5-chloro-7-((2S,5R)-4-((4-chlorophenyl)((S)-2,2-difluorocyclopropyl)methyl)-2,5-dimethylpiperazin-1-yl)-3H-imidazo[4,5-b]pyridine (Step 3), (S)-(tetrahydrofuran-2-yl)methyl methanesulfonate (Intermediate 50, 0.135 g, 0.750 mmol), and cesium carbonate (0.489, 1.50 mmol) in MeCN (1.0 mL) was stirred at 95° C. for 4 h. The mixture was cooled to room temperature, filtered, and concentrated in vacuo. The residue was purified by flash column chromatography (24 g SiO₂, EtOAc/hexanes) to give the title compound (44.9 mg, 27% yield) as a white solid. LC-MS calculated for C₂₇H₃₂Cl₂F₂N₅O (M+H)⁺: m/z=550.2. found 550.2.

### Intermediate 53. 5-Chloro-7-((2S,5R)-4-(1-(4-chlorophenyl)-3-methylbutyl)-2,5-dimethylpiperazin-1-yl)-3-(((S)-tetrahydrofuran-2-yl)methyl)-3H-imidazo[4,5-b]pyridine

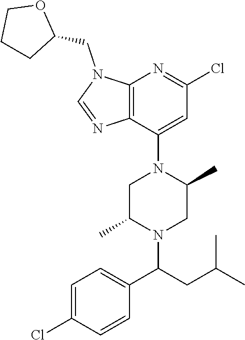 [SMILES: CC(C)CC(c1ccc(Cl)cc1)N1C[C@H](C)N(c2cc(Cl)nc3c2ncn3C[C@@H]2CCCO2)C[C@H]1C]

The title compound was prepared according to the procedures outlined in Intermediate 52, with (2R,5S)-1-(1-(4-chlorophenyl)-3-methylbutyl)-2,5-dimethylpiperazine hydrochloride (Intermediate 10) replacing (2R,5S)-1-((4-chlorophenyl)((S)-2,2-difluorocyclopropyl)methyl)-2,5-dimethylpiperazine hydrochloride in Step 1. The title compound was isolated as a single stereoisomer. LC-MS calculated for C₂₈H₃₈Cl₂N₅O (M+H)⁺: m/z=530.2. found 530.2.

### Intermediate 54. tert-Butyl (2S,5R)-4-(4-chlorobenzoyl)-2,5-dimethylpiperazine-1-carboxylate

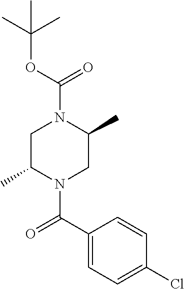 [SMILES: C[C@@H]1CN(C(=O)OC(C)(C)C)[C@@H](C)CN1C(=O)c1ccc(Cl)cc1]

The title compound was prepared according to the procedure outlined for Intermediate 9, with 4-chlorobenzoyl chloride (Aldrich 111902) replacing isovaleryl chloride. LC-MS calculated for C₁₄H₁₈ClN₂O₃(M-C₄H₈+H)⁺: m/z=297.1. found 297.1.

### Intermediate 55. (2R,5S)-1-(1-(4-Chlorophenyl)propyl)-2,5-dimethylpiperazine Hydrochloride

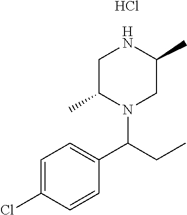 [SMILES: Cl ; CCC(c1ccc(Cl)cc1)N1C[C@H](C)NC[C@H]1C]

The title compound was prepared according to the procedures outlined for Intermediate 10, with tert-butyl (2S,5R)-4-(4-chlorobenzoyl)-2,5-dimethylpiperazine-1-carboxylate (Intermediate 54) replacing tert-butyl (2S,5R)-2,5-dimethyl-4-(3-methylbutanoyl)piperazine-1-carboxylate and ethylmagnesium bromide (Aldrich 752126) replacing (4-chlorophenyl)magnesium bromide. The title compound was isolated as a mixture of diastereomers. LC-MS calculated for C₁₅H₂₄ClN₂ (M+H)⁺: m/z=267.2. found 267.2.

### Intermediate 56. tert-Butyl (2S,5R)-2,5-dimethyl-4-(4-(trifluoromethyl)benzoyl)piperazine-1-carboxylate

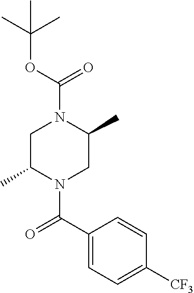 [SMILES: C[C@@H]1CN(C(=O)OC(C)(C)C)[C@@H](C)CN1C(=O)c1ccc(C(F)(F)F)cc1]

A mixture of tert-butyl (2S,5R)-2,5-dimethylpiperazine-1-carboxylate (2.00 g, 9.33 mmol, Combi-Blocks OR-8588), N,N-diisopropylethylamine (3.26 mL, 18.7 mmol), 4-(trifluoromethyl)benzoic acid (1.95 g, 10.3 mmol), and HATU (3.73 g, 9.80 mmol, Combi-Blocks OR-0618) in MeCN (12 mL) was stirred at rt overnight. The reaction mixture was concentrated in vacuo. The crude material was purified by flash column chromatography (120 g SiO₂, EtOAc/hexanes) to afford the desired product (3.35 g, 93% yield) as a white solid. LC-MS calculated for C₁₅H₁₈F₃N₂O₃(M-C₄H₈+H)⁺: m/z=331.1. found 331.2.

### Intermediate 57. (2R,5S)-1-(1-(4-(Trifluoromethyl)phenyl)propyl)-2,5-dimethylpiperazine Hydrochloride

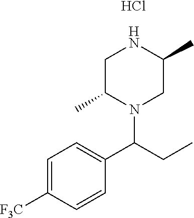 [SMILES: Cl ; CCC(c1ccc(C(F)(F)F)cc1)N1C[C@H](C)NC[C@H]1C]

The title compound was prepared according to the procedures outlined for Intermediate 10, with tert-butyl (2S,5R)-2,5-dimethyl-4-(4-(trifluoromethyl)benzoyl)piperazine-1-carboxylate (Intermediate 56) replacing tert-butyl (2S,5R)-2,5-dimethyl-4-(3-methylbutanoyl)piperazine-1-carboxylate and ethylmagnesium bromide (Aldrich 752126) replacing (4-chlorophenyl)magnesium bromide. The title compound was isolated as a mixture of diastereomers. LC-MS calculated for C₁₆H₂₄F₃N₂ (M+H)⁺: m/z=301.2. found 301.2.

### Intermediate 58. 5-Chloro-7-((2S,5R)-4-((4-chlorophenyl)(3,3-difluorocyclobutyl)methyl)-2,5-dimethylpiperazin-1-yl)-3-(((S)-tetrahydrofuran-2-yl)methyl)-3H-imidazo[4,5-b]pyridine

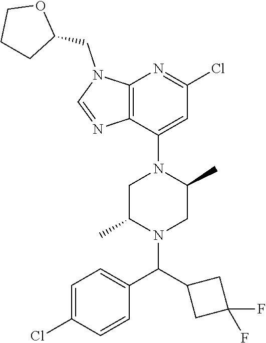 [SMILES: C[C@@H]1CN(c2cc(Cl)nc3c2ncn3C[C@@H]2CCCO2)[C@@H](C)CN1C(c1ccc(Cl)cc1)C1CC(F)(F)C1]

The title compound was prepared according to the procedures outlined in Intermediate 52, with (2R,5S)-1-((4-chlorophenyl)(3,3-difluorocyclobutyl)methyl)-2,5-dimethylpiperazine hydrochloride (Intermediate 39) replacing (2R,5S)-1-((4-chlorophenyl)((S)-2,2-difluorocyclopropyl)methyl)-2,5-dimethylpiperazine hydrochloride in Step 1. The title compound was isolated as a mixture of diastereomers. LC-MS calculated for C₂₈H₃₄Cl₂F₂N₅O (M+H)⁺: m/z=564.2. found 564.1.

### Intermediate 59. 5-Chloro-7-((2S,5R)-4-((4-chlorophenyl)(3,3-difluorocyclobutyl)methyl)-2,5-dimethylpiperazin-1-yl)-2-methyl-3-(((S)-tetrahydrofuran-2-yl)methyl)-3H-imidazo[4,5-b]pyridine

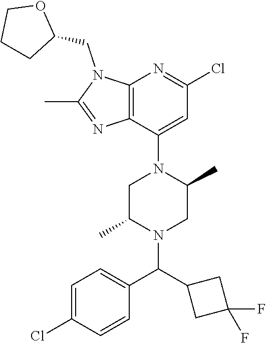 [SMILES: Cc1nc2c(N3C[C@@H](C)N(C(c4ccc(Cl)cc4)C4CC(F)(F)C4)C[C@@H]3C)cc(Cl)nc2n1C[C@@H]1CCCO1]

The title compound was prepared according to the procedures outlined in Intermediate 52, with (2R,5S)-1-((4-chlorophenyl)(3,3-difluorocyclobutyl)methyl)-2,5-dimethylpiperazine hydrochloride (Intermediate 39) replacing (2R,5s)-1-((4-chlorophenyl)((S)-2,2-difluorocyclopropyl)methyl)-2,5-dimethylpiperazine hydrochloride in Step 1 and triethyl orthoacetate replacing triethyl orthoformate in Step 3. The title compound was isolated as a mixture of diastereomers. LC-MS calculated for C₂₉H₃₆Cl₂F₂N₅O (M+H)⁺: m/z=578.2. found 578.3.

### Intermediate 60. tert-Butyl (2S,5R)-2,5-dimethyl-4-(6-(trifluoromethyl)quinoline-2-carbonyl)piperazine-1-carboxylate

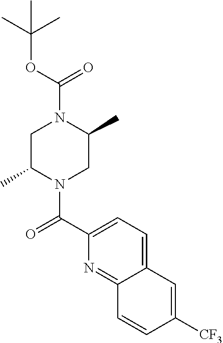 [SMILES: C[C@@H]1CN(C(=O)OC(C)(C)C)[C@@H](C)CN1C(=O)c1ccc2cc(C(F)(F)F)ccc2n1]

The title compound was prepared according to the procedures described in Intermediate 32, with 6-(trifluoromethyl)quinoline-2-carboxylic acid replacing 4-(difluoromethyl)-2-fluorobenzoic acid. LC-MS calculated for C₂₂H₂₇F₃N₃O₃(M+H)⁺: m/z=438.2. found 438.2.

### Intermediate 61. 2-(1-((2R,5S)-2,5-Dimethylpiperazin-1-yl)-2-methylpropyl)-6-(trifluoromethyl)quinoline Dihydrochloride

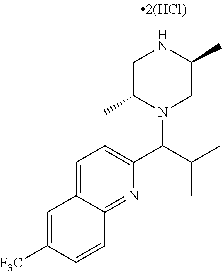 [SMILES: CC(C)C(c1ccc2cc(C(F)(F)F)ccc2n1)N1C[C@H](C)NC[C@H]1C]

The title compound was prepared according to the procedures described in Intermediate 33, with tert-butyl (2S,5R)-2,5-dimethyl-4-(6-(trifluoromethyl)quinoline-2-carbonyl)piperazine-1-carboxylate (Intermediate 60) replacing tert-butyl (2S,5R)-4-(4-(difluoromethyl)-2-fluorobenzoyl)-2,5-dimethylpiperazine-1-carboxylate in Step 1. LC-MS calculated for C₂₀H₂₇F₃N₃ (M+H)⁺: m/z=366.2. found 366.3.

### Intermediate 62. (2R,5S)-1-((4-Chloro-2,5-difluorophenyl)(3,3-difluorocyclobutyl)methyl)-2,5-dimethylpiperazine Hydrochloride

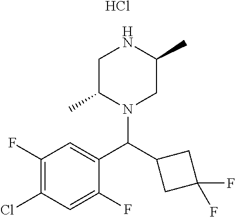 [SMILES: Cl ; C[C@@H]1CN[C@@H](C)CN1C(c1cc(F)c(Cl)cc1F)C1CC(F)(F)C1]

The title compound was prepared according to the procedures described in Intermediate 3, with 1-chloro-2,5-difluoro-4-iodobenzene replacing 1-bromo-4-(trifluoromethyl)benzene in Step 1. LC-MS calculated for C₁₇H₂₂ClF₄N₂(M+H)⁺: m/z=365.1. found 365.2.

### Intermediate 63. (2R,5S)-1-((4-Chloro-2,3-difluorophenyl)(3,3-difluorocyclobutyl)methyl)-2,5-dimethylpiperazine Hydrochloride

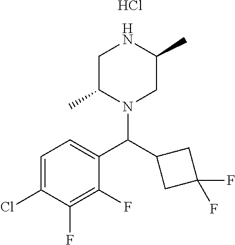 [SMILES: Cl ; C[C@@H]1CN[C@@H](C)CN1C(c1ccc(Cl)c(F)c1F)C1CC(F)(F)C1]

The title compound was prepared according to the procedures described in Intermediate 3, with 1-bromo-4-chloro-2,3-difluorobenzene replacing 1-bromo-4-(trifluoromethyl)benzene in Step 1. LC-MS calculated for C₁₇H₂₂ClF₄N₂(M+H)⁺: m/z=365.1. found 365.2.

### Intermediate 64. tert-Butyl (1R,5S)-8-(3,3-difluorocyclobutane-1-carbonyl)-3,8-diazabicyclo[3.2.1]octane-3-carboxylate

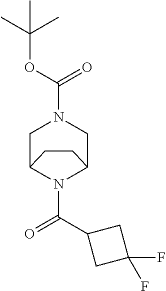 [SMILES: CC(C)(C)OC(=O)N1CC2CCC(C1)N2C(=O)C1CC(F)(F)C1]

The title compound was prepared according to the procedures described in Intermediate 2, with tert-butyl (1R,5S)-3,8-diazabicyclo[3.2.1]octane-3-carboxylate replacing tert-butyl (2S,5R)-2,5-dimethylpiperazine-1-carboxylate. LC-MS calculated for C₁₂H₁₇F₂N₂O₃(M-C₄H₈+H)⁺: m/z=275.1. found 275.2.

### Intermediate 65. (1R,5S)-8-((3,3-Difluorocyclobutyl)(4-(trifluoromethyl)phenyl)methyl)-3,8-diazabicyclo[3.2.1]octane Hydrochloride

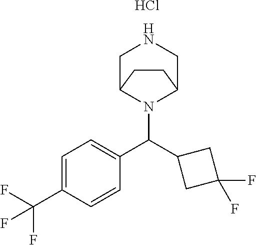 [SMILES: Cl ; FC1(F)CC(C(c2ccc(C(F)(F)F)cc2)N2C3CCC2CNC3)C1]

The title compound was prepared according to the procedures described in Intermediate 3, with tert-butyl (1R,5S)-8-(3,3-difluorocyclobutane-1-carbonyl)-3,8-diazabicyclo[3.2.1]octane-3-carboxylate (Intermediate 64) replacing tert-butyl (2S,5R)-4-(3,3-difluorocyclobutane-1-carbonyl)-2,5-dimethylpiperazine-1-carboxylate in Step 2. LC-MS calculated for C₁₈H₂₂F₅N₂ (M+H)⁺: m/z=361.2. found 361.2.

### Intermediate 66. (1R,5S)-8-(Bis(4-fluorophenyl)methyl)-3,8-diazabicyclo[3.2.1]octane Hydrochloride

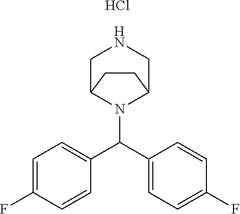 [SMILES: Cl ; Fc1ccc(C(c2ccc(F)cc2)N2C3CCC2CNC3)cc1]

The title compound was prepare according to the procedures described in Intermediate 49, with tert-butyl (1R,5S)-3,8-diazabicyclo[3.2.1]octane-3-carboxylate replacing tert-butyl (2S,5R)-2,5-dimethylpiperazine-1-carboxylate in Step 1. LC-MS calculated for C₁₉H₂₁F₂N₂ (M+H)⁺: m/z=315.2. found 315.2.

### Example 1. 4-((2S,5R)-4-((3,3-Difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine

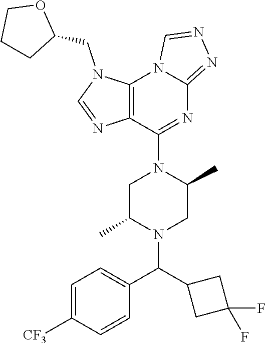 [SMILES: C[C@@H]1CN(c2nc3nncn3c3c2ncn3C[C@@H]2CCCO2)[C@@H](C)CN1C(c1ccc(C(F)(F)F)cc1)C1CC(F)(F)C1]

### Step 1. 2-Chloro-6-((2S,5R)-4-((3,3-difluorocyclobutyl)(4-(trifluoromethyl)phenyl)methyl)-2,5-dimethylpiperazin-1-yl)-9-(((S)-tetrahydrofuran-2-yl)methyl)-9H-purine

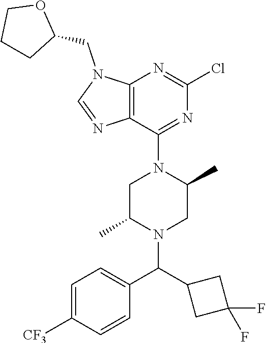 [SMILES: C[C@@H]1CN(c2nc(Cl)nc3c2ncn3C[C@@H]2CCCO2)[C@@H](C)CN1C(c1ccc(C(F)(F)F)cc1)C1CC(F)(F)C1]

To a mixture of (S)-2,6-dichloro-9-((tetrahydrofuran-2-yl)methyl)-9H-purine (Intermediate 1, 0.100 g, 0.366 mmol) and (2R,5S)-1-((3,3-difluorocyclobutyl)(4-(trifluoromethyl)phenyl)methyl)-2,5-dimethylpiperazine hydrochloride (Intermediate 3, 0.146 g, 0.366 mmol) in 1-butanol (4 mL) was added N,N-diisopropylethylamine (0.192 mL, 1.10 mmol) and the mixture was stirred at 90° C. overnight. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₂₈H₃₃ClF₅N₆O (M+H)⁺. m/z=599.2. found 599.4.

### Step 2. 6-((2S,5R)-4-((3,3-Difluorocyclobutyl)(4-(trifluoromethyl)phenyl)methyl)-2,5-dimethylpiperazin-1-yl)-2-hydrazineyl-9-(((S)-tetrahydrofuran-2-yl)methyl)-9H-purine

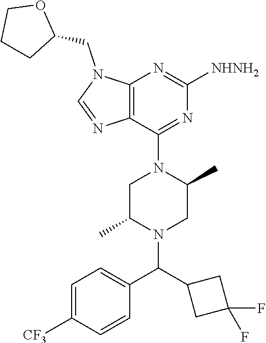 [SMILES: C[C@@H]1CN(c2nc(NN)nc3c2ncn3C[C@@H]2CCCO2)[C@@H](C)CN1C(c1ccc(C(F)(F)F)cc1)C1CC(F)(F)C1]

To a mixture of 2-chloro-6-((2S,5R)-4-((3,3-difluorocyclobutyl)(4-(trifluoromethyl)phenyl)methyl)-2,5-dimethylpiperazin-1-yl)-9-(((S)-tetrahydrofuran-2-yl)methyl)-9H-purine (Step 1) in 1,4-dioxane (4 mL) was added hydrazine (0.057 mL, 1.83 mmol), and the mixture was stirred at 120° C. for 2 h. After cooling to rt, the reaction mixture was concentrated, and the crude residue was purified by flash column chromatography (12 g SiO₂, 0-5% MeOH/CH₂Cl₂) to afford the desired product (84.4 mg, 39% yield over 2 steps) as a mixture of diastereomers in the form of an off-white solid. LC-MS calculated for C₂₈H₃₆F₅N₈O (M+H)⁺: m/z=595.3. found 595.5.

### Step 3. 4-((2S,5R)-4-((3,3-Difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine

To a mixture of 6-((2S,5R)-4-((3,3-difluorocyclobutyl)(4-(trifluoromethyl)phenyl)methyl)-2,5-dimethylpiperazin-1-yl)-2-hydrazineyl-9-(((S)-tetrahydrofuran-2-yl)methyl)-9H-purine (84.4 mg, 0.142 mmol) in AcOH (2.10 mL, 36.6 mmol) was added triethyl orthoformate (0.122 mL, 0.732 mmol) and the reaction mixture was stirred at 90° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₂₉H₃₄F₅N₈O (M+H)⁺: m/z=605.3. found 605.3. ¹H NMR (600 MHz, DMSO-d₆) (mixture of rotamers) δ 9.54 (s, 1H), 8.47-8.25 (m, 1H), 7.87-7.73 (m, 2H), 7.70-7.56 (m, 2H), 6.25-6.00 (m, 0.4H), 5.90-5.57 (m, 0.6H), 5.05-4.86 (m, 0.6H), 4.85-4.73 (m, 1H), 4.65-4.45 (m, 1.4H), 4.24-4.10 (m, 1H), 3.88-3.70 (m, 1H), 3.70-3.59 (m, 1.6H), 3.59-3.52 (m, 1H), 3.44-3.30 (m, 0.4H), 3.23-3.00 (m, 1H), 2.92-2.75 (m, 2H), 2.75-2.55 (m, 2H), 2.46-2.31 (m, 1H), 2.26-2.13 (m, 1H), 2.11-1.94 (m, 2H), 1.85-1.76 (m, 1H), 1.76-1.62 (m, 2H), 1.55-1.27 (m, 3H), 1.08-0.81 (m, 3H).

### Example 2. 4-((2S,5R)-4-((3,3-Difluorocyclobutyl)(4-(difluoromethyl)-3-fluorophenyl)methyl)-2,5-dimethylpiperazin-1-yl)-1-(((S)-tetrahydrofuran-2-yl)methyl)-1H-[1,2,4]triazolo[3,4-b]purine

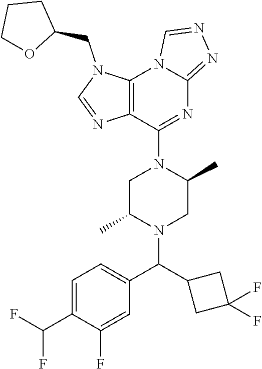 [SMILES: C[C@@H]1CN(c2nc3nncn3c3c2ncn3C[C@@H]2CCCO2)[C@@H](C)CN1C(c1ccc(C(F)F)c(F)c1)C1CC(F)(F)C1]

The title compound was prepared according to the procedures described in Example 1, with (2R,5S)-1-((3,3-difluorocyclobutyl)(4-(difluoromethyl)-3-fluorophenyl)methyl)-2,5-dimethylpiperazine hydrochloride (Intermediate 4) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₉H₃₄F₅N₈O (M+H)⁺: m/z=605.3. found 605.3. ¹H NMR (500 MHz, DMSO-d₆) (mixture of rotamers) δ 9.54 (s, 1H), 8.50-8.23 (m, 1H), 7.76-7.55 (m, 1H), 7.54-7.35 (m, 2H), 7.34-7.10 (m, 1H), 6.20-5.94 (m, 0.4H), 5.90-5.61 (m, 0.6H), 5.05-4.88 (m, 0.6H), 4.87-4.70 (m, 1H), 4.66-4.42 (m, 1.4H), 4.30-4.01 (m, 1H), 3.91-3.71 (m, 1H), 3.68-3.59 (m, 1.6H), 3.58-3.51 (m, 1H), 3.47-3.33 (m, 0.4H), 3.21-3.02 (m, 1H), 2.98-2.76 (m, 2H), 2.76-2.54 (m, 2H), 2.47-2.31 (m, 1H), 2.31-2.15 (m, 1H), 2.14-1.97 (m, 2H), 1.89-1.77 (m, 1H), 1.77-1.63 (m, 2H), 1.58-1.30 (m, 3H), 1.09-0.83 (m, 3H).

### Example 3. 4-((2S,5R)-4-((3,3-Difluorocyclobutyl)(4-(difluoromethyl)-2-fluorophenyl)methyl)-2,5-dimethylpiperazin-1-yl)-2-methyl-1-(((S)-tetrahydrofuran-2-yl)methyl)-1H-[1,2,4]triazolo[3,4-b]purine

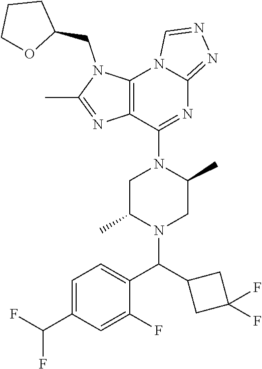 [SMILES: Cc1nc2c(N3C[C@@H](C)N(C(c4ccc(C(F)F)cc4F)C4CC(F)(F)C4)C[C@@H]3C)nc3nncn3c2n1C[C@@H]1CCCO1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (2R,5S)-1-((3,3-difluorocyclobutyl)(4-(difluoromethyl)-2-fluorophenyl)methyl)-2,5-dimethylpiperazine hydrochloride (Intermediate 6) replacing (2R,5S)-1-((3,3-difluorocyclobutyl)(4-(trifluoromethyl)phenyl)methyl)-2,5-dimethylpiperazine hydrochloride in Step 1. LC-MS calculated for C₃₀H₃₆F₅N₈O (M+H)⁺: m/z=619.3. found 619.4. ¹H NMR (500 MHz, DMSO-d₆) (mixture of rotamers) δ 9.50 (s, 1H), 7.79-7.60 (m, 1H), 7.54-7.40 (m, 2H), 7.24-6.89 (m, 1H), 6.15-5.94 (m, 0.4H), 5.89-5.63 (m, 0.6H), 5.04-4.80 (m, 0.6H), 4.80-4.66 (m, 1H), 4.62-4.45 (m, 1.4H), 4.18-3.92 (m, 2H), 3.75-3.65 (m, 1H), 3.65-3.50 (m, 1.6H), 3.42-3.20 (m, 0.4H), 3.20-3.09 (m, 1H), 2.95-2.69 (m, 3H), 2.69-2.53 (m, 4H), 2.48-2.40 (m, 1H), 2.39-2.25 (m, 1H), 2.18-2.08 (m, 1H), 2.03-1.89 (m, 2H), 1.88-1.78 (m, 1H), 1.77-1.64 (m, 1H), 1.48-1.26 (m, 3H), 1.03-0.87 (m, 3H).

### Example 4. 4-((2S,5R)-4-((3,3-Difluorocyclobutyl)(2-fluoro-4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine

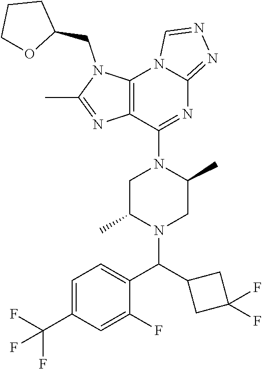 [SMILES: Cc1nc2c(N3C[C@@H](C)N(C(c4ccc(C(F)(F)F)cc4F)C4CC(F)(F)C4)C[C@@H]3C)nc3nncn3c2n1C[C@@H]1CCCO1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (2R,5S)-1-((3,3-difluorocyclobutyl)(2-fluoro-4-(trifluoromethyl)phenyl)methyl)-2,5-dimethylpiperazine hydrochloride (Intermediate 7) replacing (2R,5S)-1-((3,3-difluorocyclobutyl)(4-(trifluoromethyl)phenyl)methyl)-2,5-dimethylpiperazine hydrochloride in Step 1. LC-MS calculated for C₃₀H₃₅F₆N₈O (M+H)⁺: m/z=637.3. found 637.4. ¹H NMR (500 MHz, DMSO-d₆) (mixture of rotamers) δ 9.49 (s, 1H), 7.84-7.71 (m, 2H), 7.70-7.62 (m, 1H), 6.13-5.92 (m, 0.4H), 5.90-5.65 (m, 0.6H), 5.07-4.79 (m, 0.6H), 4.79-4.63 (m, 1H), 4.62-4.43 (m, 1.4H), 4.19-3.98 (m, 2H), 3.74-3.66 (m, 1H), 3.65-3.47 (m, 1.6H), 3.46-3.24 (m, 0.4H), 3.20-3.03 (m, 1H), 2.96-2.70 (m, 3H), 2.69-2.53 (m, 4H), 2.48-2.39 (m, 1H), 2.39-2.21 (m, 1H), 2.19-2.05 (m, 1H), 2.03-1.89 (m, 2H), 1.87-1.76 (m, 1H), 1.76-1.67 (m, 1H), 1.51-1.17 (m, 3H), 1.06-0.81 (m, 3H).

### Example 5. 4-((2S,5R)-4-((4-Chloro-2-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine

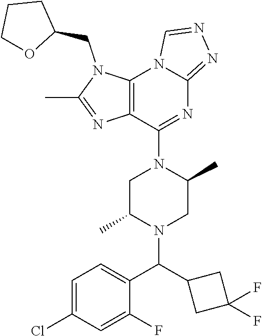 [SMILES: Cc1nc2c(N3C[C@@H](C)N(C(c4ccc(Cl)cc4F)C4CC(F)(F)C4)C[C@@H]3C)nc3nncn3c2n1C[C@@H]1CCCO1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (2R,5S)-1-((4-chloro-2-fluorophenyl)(3,3-difluorocyclobutyl)methyl)-2,5-dimethylpiperazine hydrochloride (Intermediate 8) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₉H₃₅ClF₃N₈O (M+H)⁺: m/z=603.3. found 603.3. ¹H NMR (500 MHz, DMSO-d₆) (mixture of rotamers) δ 9.50 (s, 1H), 7.60-7.53 (m, 1H), 7.51-7.46 (m, 1H), 7.41-7.33 (m, 1H), 6.10-5.97 (m, 0.4H), 5.91-5.67 (m, 0.6H), 4.99-4.81 (m, 0.6H), 4.80-4.66 (m, 1H), 4.65-4.47 (m, 1.4H), 4.20-4.07 (m, 1H), 4.06-3.91 (m, 1H), 3.79-3.66 (m, 1H), 3.66-3.50 (m, 1.6H), 3.47-3.31 (m, 0.4H), 3.24-3.05 (m, 1H), 2.99-2.70 (m, 3H), 2.70-2.54 (m, 4H), 2.48-2.39 (m, 1H), 2.38-2.25 (m, 1H), 2.22-2.08 (m, 1H), 2.04-1.88 (m, 2H), 1.88-1.78 (m, 1H), 1.78-1.66 (m, 1H), 1.54-1.26 (m, 3H), 1.11-0.92 (m, 3H).

### Example 6. 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine

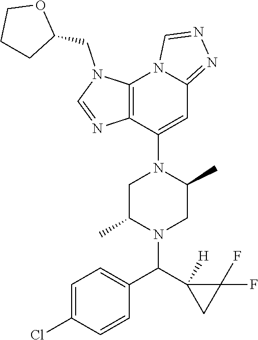 [SMILES: C[C@@H]1CN(c2cc3nncn3c3c2ncn3C[C@@H]2CCCO2)[C@@H](C)CN1C(c1ccc(Cl)cc1)[C@@H]1CC1(F)F]

### Step 1: 2-Chloro-6-((2S,5R)-4-((4-chlorophenyl)((S)-2,2-difluorocyclopropyl)methyl)-2,5-dimethylpiperazin-1-yl)-9-(((S)-tetrahydrofuran-2-yl)methyl)-9H-purine

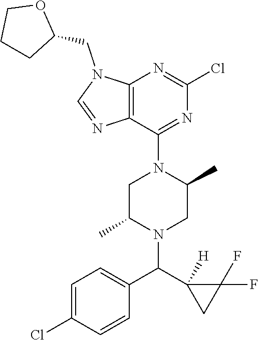 [SMILES: C[C@@H]1CN(c2nc(Cl)nc3c2ncn3C[C@@H]2CCCO2)[C@@H](C)CN1C(c1ccc(Cl)cc1)[C@@H]1CC1(F)F]

A mixture of (S)-2,6-dichloro-9-((tetrahydrofuran-2-yl)methyl)-9H-purine (Intermediate 1, 0.389 g, 1.42 mmol), (2R,5S)-1-((4-chlorophenyl)((S)-2,2-difluorocyclopropyl)methyl)-2,5-dimethylpiperazine hydrochloride (Intermediate 16, 0.500 g, 1.42 mmol), and N,N-diisopropylethylamine (0.746 mL, 4.27 mmol) in 2-propanol (3.56 mL) was stirred at 85° C. overnight. The mixture was cooled to room temperature and concentrated in vacuo. The residue was diluted with CH₂Cl₂ and quenched with water and saturated aqueous NaHCO₃. The layers were separated and the aqueous layer was extracted with CH₂Cl₂. The combined organic layers were dried over MgSO₄, concentrated in vacuo, and purified by flash column chromatography (24 g SiO₂, EtOAc/hexanes) to give the title compound (0.605 g, 1.10 mmol, 77% yield) as an off-white solid. LC-MS calculated for C₂₆H₃₁Cl₂F₂N₆O (M+H)⁺. m/z=551.2. found 551.2.

### Step 2: 6-((2S,5R)-4-((4-Chlorophenyl)((S)-2,2-difluorocyclopropyl)methyl)-2,5-dimethylpiperazin-1-yl)-2-hydrazineyl-9-(((S)-tetrahydrofuran-2-yl)methyl)-9H-purine

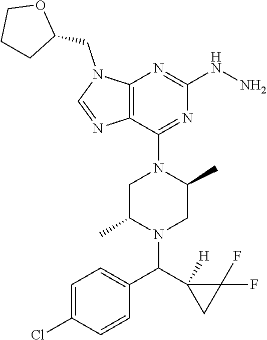 [SMILES: C[C@@H]1CN(c2nc(NN)nc3c2ncn3C[C@@H]2CCCO2)[C@@H](C)CN1C(c1ccc(Cl)cc1)[C@@H]1CC1(F)F]

A mixture of 2-chloro-6-((2S,5R)-4-((4-chlorophenyl)((S)-2,2-difluorocyclopropyl)methyl)-2,5-dimethylpiperazin-1-yl)-9-(((S)-tetrahydrofuran-2-yl)methyl)-9H-purine (Step 1) and hydrazine hydrate (0.683 mL, 10.96 mmol, Aldrich 225819) in n-BuOH (3.56 mL) was stirred at 120° C. overnight. The mixture was cooled to room temperature, diluted with CH₂Cl₂, and quenched with saturated aqueous NaHCO₃. The layers were separated and the aqueous layer was extracted with CH₂C2. The combined organic layers were dried over MgSO₄, concentrated in vacuo, and the residue was purified by flash column chromatography (24 g SiO₂, MeOH/CH₂Cl₂) to give the title compound (0.542 g, 0.991 mmol, 70% yield) as a white solid. LC-MS calculated for C₂₆H₃₄ClF₂N₈O (M+H)⁺. m/z=547.3. found 547.3.

### Step 3: 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine

A mixture of 6-((2S,5R)-4-((4-chlorophenyl)((S)-2,2-difluorocyclopropyl)methyl)-2,5-dimethylpiperazin-1-yl)-2-hydrazineyl-9-(((S)-tetrahydrofuran-2-yl)methyl)-9H-purine (Step 2) and triethyl orthoformate (0.415 mL, 2.49 mmol) in acetic acid (1.43 mL, 24.91 mmol) was stirred at 95° C. for 1 h. The mixture was concentrated in vacuo and the residue was diluted with CH₂Cl₂ and carefully quenched with saturated aqueous NaHCO₃. The layers were separated and the aqueous layer was extracted with CH₂Cl₂. The combined organic layers were dried over MgSO₄ and concentrated in vacuo. The residue was purified by flash column chromatography (24 g SiO₂, MeOH/CH₂Cl₂) to give the title compound (0.279 g, 35% yield) as a light yellow solid. The material was taken up in CH₂Cl₂ (2 mL) and Et₂O (10 mL) was added slowly. The precipitate was collected via filtration, washing with 1:1 diethyl ether/hexanes (20 mL). The solid was dried under vacuum on the filter for 30 minutes, then transferred to a vial and dried under high vacuum at 40° C. overnight. The solid material was taken up in 1:1 MeCN/H₂O (10 mL) and TFA (0.173 mL, 2.25 mmol) was added. The mixture was frozen and lyophilized to give the final product as its TFA salt. Further purification by prep-HPLC (Sunfire C18 column, eluting with a gradient of acetonitrile/water containing 0.1% TFA, at flow rate of 60 mL/min) afforded the title compound as a single stereoisomer as its TFA salt. LC-MS calculated for C₂₇H₃₂ClF₂N₈O (M+H)⁺: m/z=557.2. found 557.3. ¹H NMR (500 MHz, DMSO-d₆) (mixture of rotamers) δ 9.54 (s, 1H), 8.36 (s, 1H), 7.52-7.42 (m, 4H), 6.14-5.88 (m, 1H), 4.95-4.92 (m, 0.6H), 4.84-4.77 (m, 1.4H), 4.61-4.53 (m, 1H), 4.23-4.14 (m, 1H), 3.93-3.87 (m, 0.6H), 3.67-3.64 (m, 2.4H), 3.60-3.52 (m, 1H), 3.39-3.26 (m, 1H), 2.75-2.71 (m, 1H), 2.29-2.25 (m, 1H), 2.19-2.02 (m, 2H), 1.86-1.65 (m, 3H), 1.57-1.46 (m, 1H), 1.39-1.25 (m, 3H), 1.07-0.97 (m, 4H).

### Example 7. 4-((2S,5R)-4-((4-Chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine

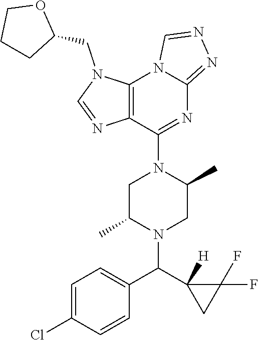 [SMILES: C[C@@H]1CN(c2nc3nncn3c3c2ncn3C[C@@H]2CCCO2)[C@@H](C)CN1C(c1ccc(Cl)cc1)[C@H]1CC1(F)F]

### Step 1: (2R,5S)-1-((4-Chlorophenyl)((R)-2,2-difluorocyclopropyl)methyl)-2,5-dimethylpiperazine Hydrochloride

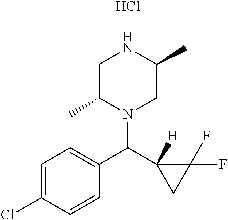 [SMILES: Cl ; C[C@@H]1CN[C@@H](C)CN1C(c1ccc(Cl)cc1)[C@H]1CC1(F)F]

A mixture of tert-butyl (2S,5R)-4-((4-chlorophenyl)((R)-2,2-difluorocyclopropyl)methyl)-2,5-dimethylpiperazine-1-carboxylate (Intermediate 19, 0.352 mmol) in THF (0.88 mL) was treated with HCl (4 M in 1,4-dioxane) (0.879 mL, 3.52 mmol) and stirred at 60° C. for 1 h. The mixture was cooled to room temperature and concentrated in vacuo and the product was used without further purification. LC-MS calculated for C₁₆H₂₂ClF₂N₂(M+H)⁺. m/z=315.1. found 315.1.

### Step 2: 2-Chloro-6-((2S,5R)-4-((4-chlorophenyl)((R)-2,2-difluorocyclopropyl)methyl)-2,5-dimethylpiperazin-1-yl)-9-(((S)-tetrahydrofuran-2-yl)methyl)-9H-purine

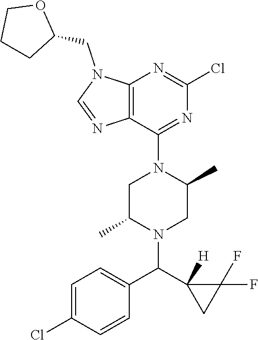 [SMILES: C[C@@H]1CN(c2nc(Cl)nc3c2ncn3C[C@@H]2CCCO2)[C@@H](C)CN1C(c1ccc(Cl)cc1)[C@H]1CC1(F)F]

A mixture of (2R,5S)-1-((4-chlorophenyl)((R)-2,2-difluorocyclopropyl)methyl)-2,5-dimethylpiperazine hydrochloride (Step 1, 0.176 mmol) and (S)-2,6-dichloro-9-((tetrahydrofuran-2-yl)methyl)-9H-purine (Intermediate 1, 48.0 mg, 0.176 mmol), and N,N-diisopropylethylamine (0.154 mL, 0.879 mmol) in 2-propanol (0.44 mL) was stirred at 90° C. overnight. The mixture cooled to room temperature, concentrated in vacuo, and directly purified by flash column chromatography (12 g SiO₂, EtOAc/hexanes) to afford the major diastereomer of the title compound as a single stereoisomer. LC-MS calculated for C₂₆H₃₁Cl₂F₂N₆O (M+H)⁺: m/z=551.2. found 551.3.

### Step 3: 6-((2S,5R)-4-((4-Chlorophenyl)((R)-2,2-difluorocyclopropyl)methyl)-2,5-dimethylpiperazin-1-yl)-2-hydrazineyl-9-(((S)-tetrahydrofuran-2-yl)methyl)-9H-purine

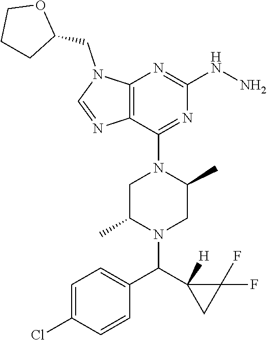 [SMILES: C[C@@H]1CN(c2nc(NN)nc3c2ncn3C[C@@H]2CCCO2)[C@@H](C)CN1C(c1ccc(Cl)cc1)[C@H]1CC1(F)F]

A mixture of 2-chloro-6-((2S,5R)-4-((4-chlorophenyl)((R)-2,2-difluorocyclopropyl)methyl)-2,5-dimethylpiperazin-1-yl)-9-(((S)-tetrahydrofuran-2-yl)methyl)-9H-purine (Step 2, 72.5 μmol) and hydrazine hydrate (45.2 μL, 0.725 mmol, Aldrich 225819) in n-BuOH (0.18 mL) was stirred at 120° C. overnight. The mixture was cooled to room temperature, concentrated in vacuo, and the residue was directly purified by flash column chromatography (12 g SiO₂, MeOH/CH₂Cl₂) to give the title compound as a clear oil. LC-MS calculated for C₂₆H₃₄ClF₂N₈O (M+H)⁺: m/z=547.3. found 547.3.

### Step 4: 4-((2S,5R)-4-((4-Chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine

A mixture of 6-((2S,5R)-4-((4-chlorophenyl)((R)-2,2-difluorocyclopropyl)methyl)-2,5-dimethylpiperazin-1-yl)-2-hydrazineyl-9-(((S)-tetrahydrofuran-2-yl)methyl)-9H-purine (Step 3) and triethyl orthoformate (27.8 μL, 0.167 mmol) in acetic acid (96.0 μL, 1.67 mmol) was stirred at 95° C. for 1 h. The mixture was cooled to room temperature, diluted with MeOH, and purified by prep-HPLC (Sunfire C18 column, eluting with a gradient of acetonitrile/water containing 0.1% TFA, at flow rate of 60 mL/min) to afford the title compound as a single stereoisomer as its TFA salt. LC-MS calculated for C₂₇H₃₂ClF₂N₈O (M+H)⁺: m/z=557.2. found 557.3. ¹H NMR (500 MHz, DMSO-d₆) (mixture of rotamers) δ 9.57 (s, 1H), 8.40-8.35 (m, 1H), 7.51 (d, J=8.8 Hz, 2H), 7.46 (d, J=8.1 Hz, 2H), 6.10-5.96 (m, 1H), 4.97-4.93 (m, 0.5H), 4.85-4.79 (m, 1.5H), 4.59 (dd, J=15.1, 7.7 Hz, 1H), 4.19 (qd, J=7.1, 2.8 Hz, 1H), 3.98-3.88 (m, 0.5H), 3.70-3.62 (m, 2.5H), 3.60-3.53 (m, 1H), 3.48-3.32 (m, 1H), 2.85-2.62 (m, 1H), 2.39-2.01 (m, 3H), 1.86-1.64 (m, 3H), 1.60-1.50 (m, 1H), 1.37-1.24 (m, 3H), 1.08-0.97 (m, 4H).

### Example 8. 4-((2S,5R)-4-((4-Chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine

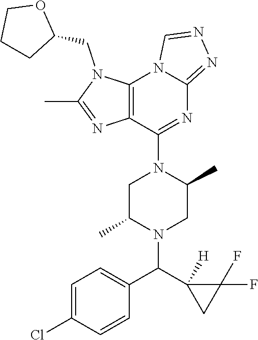 [SMILES: Cc1nc2c(N3C[C@@H](C)N(C(c4ccc(Cl)cc4)[C@@H]4CC4(F)F)C[C@@H]3C)nc3nncn3c2n1C[C@@H]1CCCO1]

The title compound was prepared according to the procedures described in Steps 2-4 for Example 7, with (2R,5S)-1-((4-chlorophenyl)((S)-2,2-difluorocyclopropyl)methyl)-2,5-dimethylpiperazine hydrochloride (Intermediate 16) replacing (2R,5S)-1-((4-chlorophenyl)((R)-2,2-difluorocyclopropyl)methyl)-2,5-dimethylpiperazine hydrochloride and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The major diastereomer of the title compound was isolated as a single stereoisomer as its TFA salt. LC-MS calculated for C₂₈H₃₄ClF₂N₈O (M+H)⁺: m/z=571.3. found 571.3. ¹H NMR (500 MHz, DMSO-d₆) (mixture of rotamers) δ 9.52 (s, 1H), 7.59-7.54 (m, 2H), 7.49-7.45 (m, 2H), 6.44-6.40 (m, 0.5H), 5.91-5.79 (m, 0.5H), 5.23-5.20 (m, 0.5H), 4.78-4.69 (m, 1H), 4.61-4.52 (m, 1.5H), 4.16-4.08 (m, 1H), 3.76-3.64 (m, 1.5H), 3.60-3.53 (m, 1H), 3.48-3.41 (m, 0.5H), 3.29-3.24 (m, 1H), 3.08-3.00 (m, 1H), 2.94-2.87 (m, 2H), 2.65 (s, 1.5H), 2.56 (s, 1.5H), 2.17-2.12 (m, 1H), 2.07-1.91 (m, 2H), 1.89-1.79 (m, 1H), 1.78-1.68 (m, 1H), 1.57 (d, J=6.5 Hz, 1.5H), 1.51-1.46 (m, 2.5H), 1.13-1.04 (m, 1H), 0.92-0.84 (m, 3H).

### Example 9. 4-((2S,5R)-4-(1-(4-Chlorophenyl)-3-methylbutyl)-2,5-dimethylpiperazin-1-yl)-1-(((S)-tetrahydrofuran-2-yl)methyl)-1H-[1,2,4]triazolo[3,4-b]purine

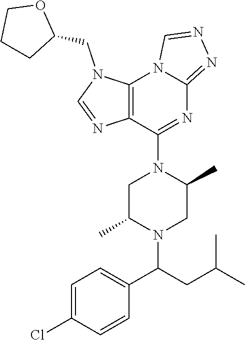 [SMILES: CC(C)CC(c1ccc(Cl)cc1)N1C[C@H](C)N(c2nc3nncn3c3c2ncn3C[C@@H]2CCCO2)C[C@H]1C]

The title compound was prepared according to the procedures described in Example 6, with (2S,5R)-4-(1-(4-chlorophenyl)-3-methylbutyl)-2,5-dimethylpiperazine hydrochloride (Intermediate 10) replacing (2R,5S)-1-((4-chlorophenyl)((S)-2,2-difluorocyclopropyl)methyl)-2,5-dimethylpiperazine hydrochloride. Following flash column chromatography in Step 3, the material obtained was purified by prep-HPLC (Sunfire C18 column, eluting with a gradient of acetonitrile/water containing 0.1% TFA, at flow rate of 60 mL/min) to afford the title compound as a single stereoisomer as its TFA salt. LC-MS calculated for C₂₈H₃₈ClN₈O (M+H)⁺: m/z=537.3. found 537.3. ¹H NMR (500 MHz, DMSO-d₆) δ 9.47 (s, 1H), 8.31 (s, 1H), 7.60-7.37 (m, 4H), 5.81-4.95 (m, 2H), 4.80 (dd, J=15.0, 3.0 Hz, 1H), 4.58 (dd, J=15.0, 7.5 Hz, 1H), 4.22 (qd, J=7.1, 2.9 Hz, 1H), 4.03-3.80 (m, 1H), 3.74-3.51 (m, 3H), 3.40-2.76 (m, 3H), 2.11 (dtd, J=12.5, 7.3, 4.9 Hz, 1H), 1.90-1.66 (m, 5H), 1.42 (d, J=6.4 Hz, 3H), 1.28-1.19 (m, 1H), 1.12-1.02 (m, 3H), 0.88 (d, J=6.5 Hz, 3H), 0.82 (d, J=6.6 Hz, 3H).

### Example 10. 4-((2S,5R)-4-((4-Chlorophenyl)(cyclopropyl)methyl)-2,5-dimethylpiperazin-1-yl)-1-(((S)-tetrahydrofuran-2-yl)methyl)-1H-[1,2,4]triazolo[3,4-b]purine

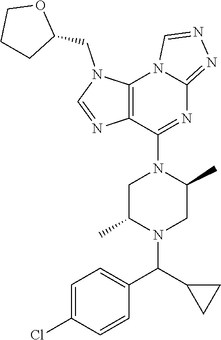 [SMILES: C[C@@H]1CN(c2nc3nncn3c3c2ncn3C[C@@H]2CCCO2)[C@@H](C)CN1C(c1ccc(Cl)cc1)C1CC1]

The title compound was prepared according to the procedures described for Example 7, with tert-butyl (2S,5R)-4-((4-chlorophenyl)(cyclopropyl)methyl)-2,5-dimethylpiperazine-1-carboxylate (Intermediate 14) replacing tert-butyl (2S,5R)-4-((4-chlorophenyl)((R)-2,2-difluorocyclopropyl)methyl)-2,5-dimethylpiperazine-1-carboxylate. The title compound was isolated as a mixture of diastereomers as the TFA salts. LC-MS calculated for C₂₇H₃₄ClN₈O (M+H)⁺: m/z=521.3. found 521.3.

### Examples 11 and 12. 4-((2S,5R)-4-((3R)-1-(4-Chlorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine and 4-((2S,5R)-4-((3S)-1-(4-chlorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine

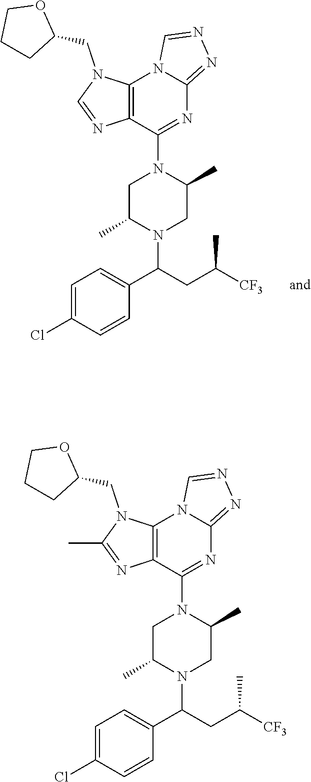 [SMILES: C[C@@H]1CN(c2nc3nncn3c3c2ncn3C[C@@H]2CCCO2)[C@@H](C)CN1C(C[C@@H](C)C(F)(F)F)c1ccc(Cl)cc1 ; Cc1nc2c(N3C[C@@H](C)N(C(C[C@H](C)C(F)(F)F)c4ccc(Cl)cc4)C[C@@H]3C)nc3nncn3c2n1C[C@@H]1CCCO1]

The title compounds were prepared according to the procedures described for Example 7, with tert-butyl (2S,5R)-4-(1-(4-chlorophenyl)-4,4,4-trifluoro-3-methylbutyl)-2,5-dimethylpiperazine-1-carboxylate (Intermediate 21) replacing tert-butyl (2S,5R)-4-((4-chlorophenyl)((R)-2,2-difluorocyclopropyl)methyl)-2,5-dimethylpiperazine-1-carboxylate. Separation of the diastereomers was achieved following prep-HPLC purification (Sunfire C18 column, eluting with a gradient of acetonitrile/water containing 0.1% TFA, at flow rate of 60 mL/min) to afford each of the title compounds as a single stereoisomer as its TFA salt.

Example 11: Retention time on LC-MS tᵣ=1.276 min, LC-MS calculated for C₂₈H₃₅ClF₃N₈O (M+H)⁺: m/z=591.3. found 591.3.

Example 12: Retention time on LC-MS tᵣ=1.347 min, LC-MS calculated for C₂₈H₃₅ClF₃N₈O (M+H)⁺: m/z=591.3. found 591.3.

### Example 13. 4-((2S,5R)-4-(((S)-2,2-Difluorocyclopropyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine

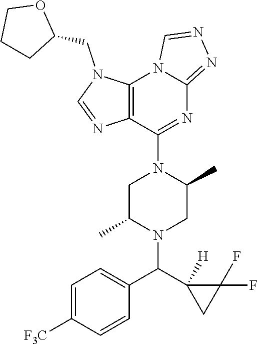 [SMILES: C[C@@H]1CN(c2nc3nncn3c3c2ncn3C[C@@H]2CCCO2)[C@@H](C)CN1C(c1ccc(C(F)(F)F)cc1)[C@@H]1CC1(F)F]

The title compound was prepared according to the procedures described for Example 7, with tert-butyl (2S,5R)-4-(((S)-2,2-difluorocyclopropyl)(4-(trifluoromethyl)phenyl)methyl)-2,5-dimethylpiperazine-1-carboxylate (Intermediate 17) replacing tert-butyl (2S,5R)-4-((4-chlorophenyl)((R)-2,2-difluorocyclopropyl)methyl)-2,5-dimethylpiperazine-1-carboxylate. The title compound was isolated as a single stereoisomer as its TFA salt. LC-MS calculated for C₂₈H₃₂F₅N₈O (M+H)⁺. m/z=591.3. found 591.3.

### Example 14. 4-((2S,5R)-2,5-Dimethyl-4-(3-methyl-1-(4-(trifluoromethyl)phenyl)butyl)piperazin-1-yl)-1-(((S)-tetrahydrofuran-2-yl)methyl)-1H-[1,2,4]triazolo[3,4-b]purine

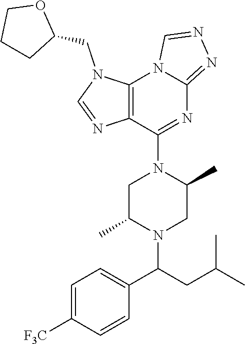 [SMILES: CC(C)CC(c1ccc(C(F)(F)F)cc1)N1C[C@H](C)N(c2nc3nncn3c3c2ncn3C[C@@H]2CCCO2)C[C@H]1C]

The title compound was prepared according to the procedures described in Steps 2-4 for Example 7, with (2R,5S)-2,5-dimethyl-1-(3-methyl-1-(4-(trifluoromethyl)phenyl)butyl)piperazine hydrochloride (Intermediate 11) replacing (2R,5S)-1-((4-chlorophenyl)((R)-2,2-difluorocyclopropyl)methyl)-2,5-dimethylpiperazine hydrochloride. The title compound was isolated as a single stereoisomer as its TFA salt. LC-MS calculated for C₂₉H₃₈F₃N₈O (M+H)⁺: m/z=571.3. found 571.3.

### Example 15. 4-((2S,5R)-4-(1-(4-Chloro-3-fluorophenyl)-3-methylbutyl)-2,5-dimethylpiperazin-1-yl)-1-(((S)-tetrahydrofuran-2-yl)methyl)-1H-[1,2,4]triazolo[3,4-b]purine

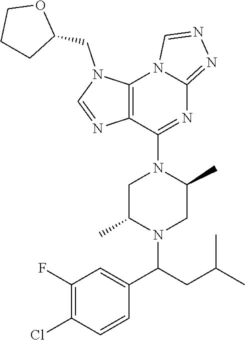 [SMILES: CC(C)CC(c1ccc(Cl)c(F)c1)N1C[C@H](C)N(c2nc3nncn3c3c2ncn3C[C@@H]2CCCO2)C[C@H]1C]

The title compound was prepared according to the procedures described in Steps 2-4 for Example 7, with (2R,5S)-1-(1-(4-chloro-3-fluorophenyl)-3-methylbutyl)-2,5-dimethylpiperazine hydrochloride (Intermediate 12) replacing (2R,5S)-1-((4-chlorophenyl)((R)-2,2-difluorocyclopropyl)methyl)-2,5-dimethylpiperazine hydrochloride. The title compound was isolated as a single stereoisomer as its TFA salt. LC-MS calculated for C₂₈H₃₇ClFN₈O (M+H)⁺: m/z=555.3. found 555.3.

### Examples 16 and 17. 4-((2S,5R)-4-((3R)-1-(4-Chloro-3-fluorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine and 4-((2S,5R)-4-((3S)-1-(4-chloro-3-fluorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine

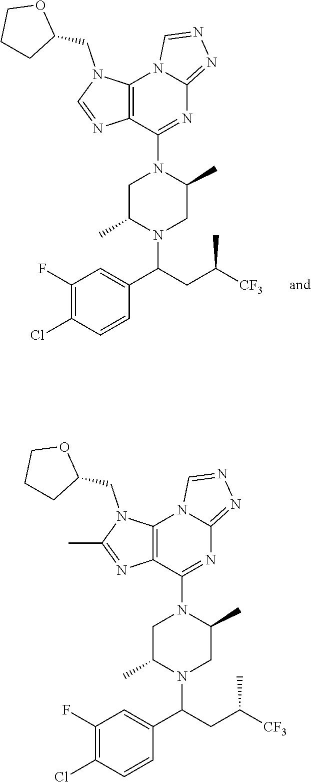 [SMILES: C[C@@H]1CN(c2nc3nncn3c3c2ncn3C[C@@H]2CCCO2)[C@@H](C)CN1C(C[C@@H](C)C(F)(F)F)c1ccc(Cl)c(F)c1 ; Cc1nc2c(N3C[C@@H](C)N(C(C[C@H](C)C(F)(F)F)c4ccc(Cl)c(F)c4)C[C@@H]3C)nc3nncn3c2n1C[C@@H]1CCCO1]

The title compounds were prepared according to the procedures described in Steps 2-4 for Example 7, with (2R,5S)-1-(1-(4-chloro-3-fluorophenyl)-4,4,4-trifluoro-3-methylbutyl)-2,5-dimethylpiperazine hydrochloride (Intermediate 22) replacing (2R,5S)-1-((4-chlorophenyl)((R)-2,2-difluorocyclopropyl)methyl)-2,5-dimethylpiperazine hydrochloride. Separation of the diastereomers was achieved following prep-HPLC purification (Sunfire C18 column, eluting with a gradient of acetonitrile/water containing 0.1% TFA, at flow rate of 60 mL/min) to afford each of the title compounds as a single stereoisomer as its TFA salt.

Example 16: Retention time on LC-MS tᵣ=1.415 min, LC-MS calculated for C₂₈H₃₄ClF₄N₈O (M+H)⁺: m/z=609.3. found 609.2.

Example 17: Retention time on LC-MS tᵣ=1.479 min, LC-MS calculated for C₂₈H₃₄ClF₄N₈O (M+H)⁺: m/z=609.3. found 609.2.

### Example 18. 4-((2S,5R)-4-(1-(4-Chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine

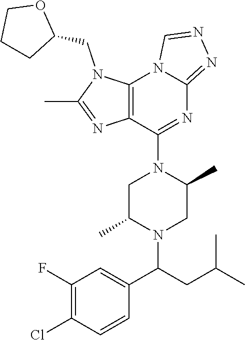 [SMILES: Cc1nc2c(N3C[C@@H](C)N(C(CC(C)C)c4ccc(Cl)c(F)c4)C[C@@H]3C)nc3nncn3c2n1C[C@@H]1CCCO1]

The title compound was prepared according to the procedures described in Steps 2-4 for Example 7, with (2R,5S)-1-(1-(4-chloro-3-fluorophenyl)-3-methylbutyl)-2,5-dimethylpiperazine hydrochloride (Intermediate 12) replacing (2R,5S)-1-((4-chlorophenyl)((R)-2,2-difluorocyclopropyl)methyl)-2,5-dimethylpiperazine hydrochloride and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The title compound was isolated as a single stereoisomer as its TFA salt. LC-MS calculated for C₂₉H₃₉ClFN₈O (M+H)⁺. m/z=569.3. found 569.2.

### Example 19. 4-((2S,5R)-4-(Bis(5-(trifluoromethyl)pyridin-2-yl)methyl)-2,5-dimethylpiperazin-1-yl)-1-(((S)-tetrahydrofuran-2-yl)methyl)-1H-[1,2,4]triazolo[3,4-b]purine

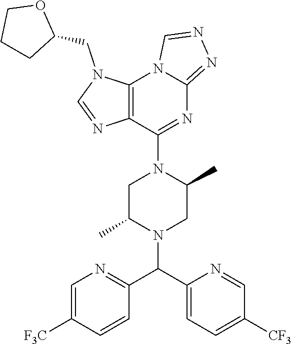 [SMILES: C[C@@H]1CN(c2nc3nncn3c3c2ncn3C[C@@H]2CCCO2)[C@@H](C)CN1C(c1ccc(C(F)(F)F)cn1)c1ccc(C(F)(F)F)cn1]

The title compound was prepared according to the procedures described in Example 6, with (2R,5S)-1-(bis(5-(trifluoromethyl)pyridin-2-yl)methyl)-2,5-dimethylpiperazine dihydrochloride (Intermediate 24) replacing (2R,5S)-1-((4-chlorophenyl)((S)-2,2-difluorocyclopropyl)methyl)-2,5-dimethylpiperazine hydrochloride. Following flash column chromatography in Step 3, the material obtained was purified by prep-HPLC (Sunfire C18 column, eluting with a gradient of acetonitrile/water containing 0.1% TFA, at flow rate of 60 mL/min) to afford the title compound as its TFA salt. LC-MS calculated for C₃₀H₃₁F₆N₁₀O (M+H)⁺: m/z=661.3. found 661.2. ¹H NMR (500 MHz, DMSO-d₆) (mixture of rotamers) δ 9.54 (s, 1H), 8.97-8.93 (m, 1H), 8.89-8.85 (m, 1H), 8.40-8.31 (m, 1H), 8.31-8.23 (m, 2H), 8.11 (d, J=8.3 Hz, 1H), 8.08-8.00 (m, 1H), 6.23-6.07 (m, 0.4H), 5.98-5.88 (m, 0.6H), 5.21 (s, 1H), 5.11-5.08 (m, 0.6H), 4.83-4.73 (m, 1H), 4.71-4.63 (m, 0.4H), 4.61-4.52 (m, 1H), 4.18 (tt, J=8.6, 4.2 Hz, 1H), 3.95-3.85 (m, 0.6H), 3.69-3.61 (m, 1.4H), 3.56 (td, J=7.7, 5.8 Hz, 1H), 3.22-3.05 (m, 1H), 3.00-2.93 (m, 1H), 2.46-2.32 (m, 1H), 2.08 (ddt, J=12.2, 7.3, 4.2 Hz, 1H), 1.87-1.64 (m, 3H), 1.57-1.42 (m, 3H), 0.99 (d, J=6.5 Hz, 3H).

### Example 20. 4-((2S,5R)-4-(Bis(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine

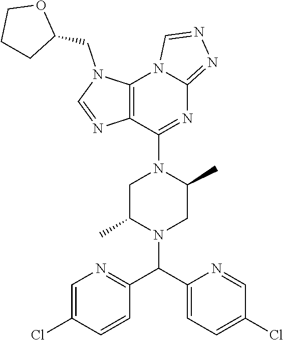 [SMILES: C[C@@H]1CN(c2nc3nncn3c3c2ncn3C[C@@H]2CCCO2)[C@@H](C)CN1C(c1ccc(Cl)cn1)c1ccc(Cl)cn1]

The title compound was prepared according to the procedures described in Steps 2-4 for Example 7, with (2R,5S)-1-(bis(5-chloropyridin-2-yl)methyl)-2,5-dimethylpiperazine dihydrochloride (Intermediate 26) replacing (2R,5S)-1-((4-chlorophenyl)((R)-2,2-difluorocyclopropyl)methyl)-2,5-dimethylpiperazine hydrochloride. The title compound was isolated as its TFA salt. LC-MS calculated for C₂₈H₃₁Cl₂N₁₀O (M+H)⁺: m/z=593.2. found 593.3.

### Examples 21 and 22. 4-((2S,5R)-4-((S)-(4-Chlorophenyl)(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine and 4-((2S,5R)-4-((R)-(4-chlorophenyl)(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine

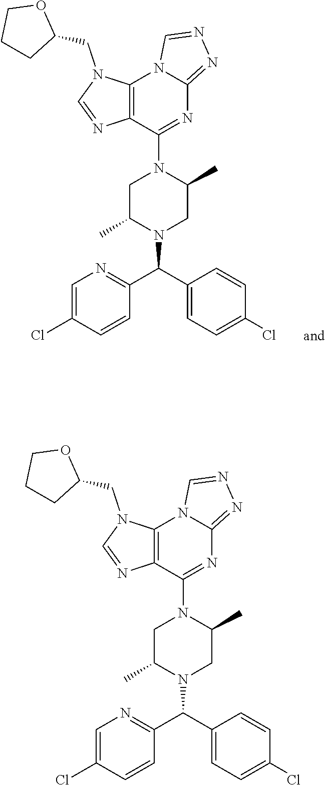 [SMILES: C[C@@H]1CN(c2nc3nncn3c3c2ncn3C[C@@H]2CCCO2)[C@@H](C)CN1[C@@H](c1ccc(Cl)cc1)c1ccc(Cl)cn1 ; C[C@@H]1CN(c2nc3nncn3c3c2ncn3C[C@@H]2CCCO2)[C@@H](C)CN1[C@H](c1ccc(Cl)cc1)c1ccc(Cl)cn1]

The title compounds were prepared according to the procedures described in Steps 2-4 for Example 7, with (2R,5S)-1-((4-chlorophenyl)(5-chloropyridin-2-yl)methyl)-2,5-dimethylpiperazine hydrochloride (Intermediate 28) replacing (2R,5S)-1-((4-chlorophenyl)((R)-2,2-difluorocyclopropyl)methyl)-2,5-dimethylpiperazine hydrochloride. Separation of the diastereomers was achieved following prep-HPLC purification (Sunfire C18 column, eluting with a gradient of acetonitrile/water containing 0.1% TFA, at flow rate of 60 mL/min) to afford each of the title compounds as a single stereoisomer as its TFA salt.

Example 21: Retention time on LC-MS tᵣ=1.345 min, LC-MS calculated for C₂₉H₃₂Cl₂N₉O (M+H)⁺: m/z=592.2. found 592.3.

Example 22: Retention time on LC-MS tᵣ=1.450 min, LC-MS calculated for C₂₉H₃₂Cl₂N₉O (M+H)⁺: m/z=592.2. found 592.3.

### Example 23. 4-((2S,5R)-4-(1-(4-Chloro-3-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine

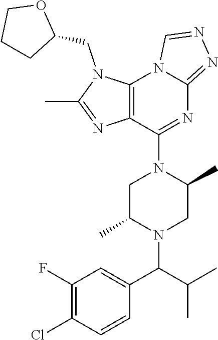 [SMILES: Cc1nc2c(N3C[C@@H](C)N(C(c4ccc(Cl)c(F)c4)C(C)C)C[C@@H]3C)nc3nncn3c2n1C[C@@H]1CCCO1]

### Step 1. 2-Chloro-6-((2S,5R)-4-(1-(4-chloro-3-fluorophenyl)-2-methylpropyl)-2,5-dimethylpiperazin-1-yl)-8-methyl-9-(((S)-tetrahydrofuran-2-yl)methyl)-9H-purine

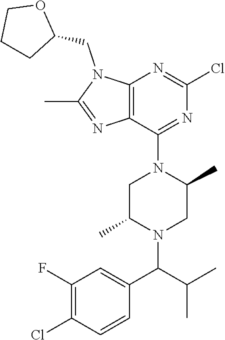 [SMILES: Cc1nc2c(N3C[C@@H](C)N(C(c4ccc(Cl)c(F)c4)C(C)C)C[C@@H]3C)nc(Cl)nc2n1C[C@@H]1CCCO1]

To a mixture of (S)-2,6-dichloro-8-methyl-9-((tetrahydrofuran-2-yl)methyl)-9H-purine (Intermediate 5, 232 mg, 0.81 mmol) and (2R,5S)-1-(1-(4-chloro-3-fluorophenyl)-2-methylpropyl)-2,5-dimethylpiperazine dihydrochloride (Intermediate 30, 300 mg, 0.81 mmol) in 1-butanol (1.6 mL) was added N,N-diisopropylethylamine (0.42 mL, 2.42 mmol) and the mixture was stirred at 85° C. for 1 h. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated in vacuo. The crude residue was purified by flash column chromatography (40 g SiO₂, CH₂Cl₂/MeOH) to give the title compound as a brown oil (255 mg, 57% yield). LC-MS calculated for C₂₇H₃₆Cl₂FN₆O (M+H)⁺: m/z=549.2. found 549.3.

### Step 2. 6-((2S,5R)-4-(1-(4-Chloro-3-fluorophenyl)-2-methylpropyl)-2,5-dimethylpiperazin-1-yl)-2-hydrazineyl-8-methyl-9-(((S)-tetrahydrofuran-2-yl)methyl)-9H-purine

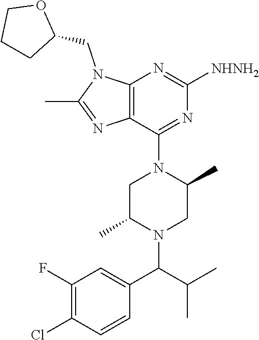 [SMILES: Cc1nc2c(N3C[C@@H](C)N(C(c4ccc(Cl)c(F)c4)C(C)C)C[C@@H]3C)nc(NN)nc2n1C[C@@H]1CCCO1]

To a mixture of 2-chloro-6-((2S,5R)-4-(1-(4-chloro-3-fluorophenyl)-2-methylpropyl)-2,5-dimethylpiperazin-1-yl)-8-methyl-9-(((S)-tetrahydrofuran-2-yl)methyl)-9H-purine (127 mg, 0.23 mmol) in 1,4-dioxane (2.3 mL) was added hydrazine (36.4 μL, 1.16 mmol), and the mixture was stirred at 120° C. overnight. After cooling to rt, the reaction mixture was concentrated under reduced pressure, and the crude residue was purified by flash column chromatography (40 g SiO₂, 0-20% MeOH/CH₂Cl₂) to afford the desired product as a brown oil. LC-MS calculated for C₂₇H₃₉ClFN₈O (M+H)⁺: m/z=545.3. found 545.4.

### Step 3. 4-((2S,5R)-4-(1-(4-Chloro-3-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine

To a mixture of 6-((2S,5R)-4-(1-(4-chloro-3-fluorophenyl)-2-methylpropyl)-2,5-dimethylpiperazin-1-yl)-2-hydrazineyl-8-methyl-9-(((S)-tetrahydrofuran-2-yl)methyl)-9H-purine (Step 2) in AcOH (0.40 mL, 7.0 mmol) was added triethyl orthoformate (116 μL, 0.70 mmol) and the reaction mixture was stirred at 95° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₂₈H₃₇ClFN₈O (M+H)⁺: m/z=555.3. found 555.3. ¹H NMR (600 MHz, DMSO-d₆) (mixture of rotamers) δ 9.49 (s, 1H), 7.65-7.54 (m, 1H), 7.41-7.32 (m, 1H), 7.22-7.12 (m, 1H), 6.10-5.92 (m, 0.4H), 5.87-5.70 (m, 0.6H), 5.00-4.84 (m, 0.6H), 4.79-4.65 (m, 1H), 4.62-4.46 (m, 1.4H), 4.17-4.06 (m, 1H), 3.74-3.67 (m, 1H), 3.67-3.53 (m, 1.6H), 3.51-3.29 (m, 1.4H), 3.07-2.95 (m, 1H), 2.92-2.80 (m, 1H), 2.70-2.53 (m, 4H), 2.35-2.24 (m, 1H), 2.18-2.09 (m, 1H), 1.98-1.90 (m, 1H), 1.89-1.79 (m, 1H), 1.78-1.68 (m, 1H), 1.52-1.26 (m, 3H), 1.02-0.86 (m, 3H), 0.87-0.64 (m, 6H).

### Example 24. 4-((2S,5R)-4-(1-(3-fluoro-4-methoxyphenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine

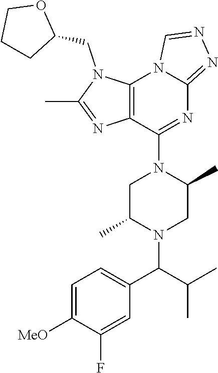 [SMILES: COc1ccc(C(C(C)C)N2C[C@H](C)N(c3nc4nncn4c4c3nc(C)n4C[C@@H]3CCCO3)C[C@H]2C)cc1F]

The title compound was prepared according to the procedures described in Example 23, with (2R,5S)-1-(1-(3-fluoro-4-methoxyphenyl)-2-methylpropyl)-2,5-dimethylpiperazine dihydrochloride (Intermediate 31) replacing (2R,5S)-1-(1-(4-chloro-3-fluorophenyl)-2-methylpropyl)-2,5-dimethylpiperazine dihydrochloride in Step 1. LC-MS calculated for C₂₉H₄₀FN₈O₂(M+H)⁺: m/z=551.3. found 551.4.

### Example 25. 4-((2S,5R)-4-(1-(4-(Difluoromethyl)-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine

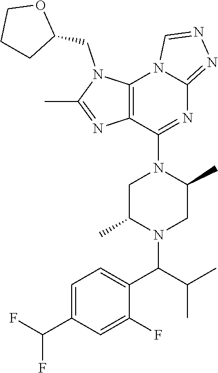 [SMILES: Cc1nc2c(N3C[C@@H](C)N(C(c4ccc(C(F)F)cc4F)C(C)C)C[C@@H]3C)nc3nncn3c2n1C[C@@H]1CCCO1]

The title compound was prepared according to the procedures described in Example 23, with (2R,5S)-1-(1-(4-(difluoromethyl)-2-fluorophenyl)-2-methylpropyl)-2,5-dimethylpiperazine dihydrochloride (Intermediate 33) replacing (2R,5S)-1-(1-(4-chloro-3-fluorophenyl)-2-methylpropyl)-2,5-dimethylpiperazine dihydrochloride in Step 1. LC-MS calculated for C₂₉H₃₈F₃N₈O (M+H)⁺: m/z=571.3. found 571.2.

### Examples 26 and 27. 4-((2S,5R)-4-((S)-1-(4-Bromo-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine and 4-((2S,5R)-4-((R)-1-(4-bromo-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine

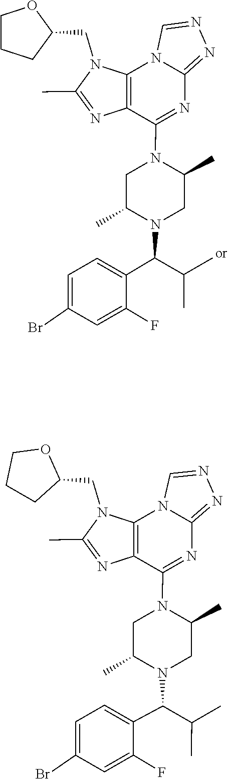

The title compounds were prepared according to the procedures described in Example 23, with (2R,5S)-1-(1-(4-bromo-2-fluorophenyl)-2-methylpropyl)-2,5-dimethylpiperazine dihydrochloride (Intermediate 35) replacing (2R,5S)-1-(1-(4-chloro-3-fluorophenyl)-2-methylpropyl)-2,5-dimethylpiperazine dihydrochloride in Step 1.

Example 26: Retention time on LC-MS tᵣ=2.48 min. LC-MS calculated for C₂₈H₃₇BrFN₈O (M+H)⁺: m/z=599.2. found 599.3. ¹H NMR (600 MHz, DMSO-d₆) (mixture of rotamers) δ 9.49 (s, 1H), 7.66-7.54 (m, 1H), 7.51-7.47 (m, 1H), 7.46-7.39 (m, 1H), 6.02-5.86 (m, 0.4H), 5.86-5.71 (m, 0.6H) 4.98-4.80 (m, 0.6H), 4.78-4.66 (m, 1H), 4.63-4.47 (m, 1.4H), 4.18-4.07 (m, 1H), 3.75-3.35 (m, 5H), 3.12-3.02 (m, 1H), 2.85-2.73 (m, 1H), 2.68-2.53 (m, 3H), 2.40-2.31 (m, 1H), 2.18-2.09 (m, 1H), 1.98-1.89 (m, 1H), 1.88-1.78 (m, 1H), 1.77-1.68 (m, 1H), 1.46-1.24 (m, 3H), 1.01-0.92 (m, 3H), 0.92-0.78 (m, 3H), 0.75-0.65 (m, 3H).

Example 27: Retention time on LC-MS tᵣ=2.58 min. LC-MS calculated for C₂₈H₃₇BrFN₈O (M+H)⁺: m/z=599.2. found 599.3.

### Examples 28 and 29. 4-((2S,5R)-4-((S)-1-(4-Chloro-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine and 4-((2S,5R)-4-((R)-1-(4-chloro-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine

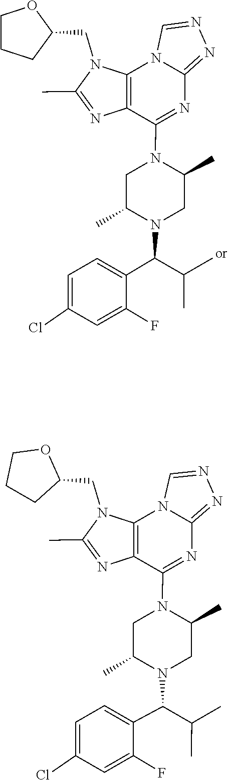

The title compounds were prepared according to the procedures described in Example 23, with (2R,5S)-1-(1-(4-chloro-2-fluorophenyl)-2-methylpropyl)-2,5-dimethylpiperazine dihydrochloride (Intermediate 37) replacing (2R,5S)-1-(1-(4-chloro-3-fluorophenyl)-2-methylpropyl)-2,5-dimethylpiperazine dihydrochloride in Step 1.

Example 28: Retention time on LC-MS tᵣ=2.42 min. LC-MS calculated for C₂₈H₃₇ClFN₈O (M+H)⁺: m/z=555.3. found 555.3. ¹H NMR (600 MHz, DMSO-d₆) (mixture of rotamers) δ 9.49 (s, 1H), 7.53-7.42 (m, 2H), 7.39-7.34 (m, 1H), 6.00-5.86 (m, 0.4H), 5.86-5.70 (m, 0.6H), 4.97-4.80 (m, 0.6H), 4.78-4.65 (m, 1H), 4.61-4.47 (m, 1.4H), 4.17-4.06 (m, 1H), 3.76-3.35 (m, 4H), 3.12-3.02 (m, 1H), 2.84-2.73 (m, 1H), 2.68-2.54 (m, 4H), 2.41-2.30 (m, 1H), 2.18-2.09 (m, 1H), 1.98-1.90 (m, 1H), 1.88-1.78 (m, 1H), 1.77-1.68 (m, 1H), 1.46-1.21 (m, 3H), 1.02-0.92 (m, 3H), 0.92-0.78 (m, 3H), 0.76-0.63 (m, 3H).

Example 29: Retention time on LC-MS tᵣ=2.52 min. LC-MS calculated for C₂₈H₃₇ClFN₈O (M+H)⁺: m/z=555.3. found 555.3.

### Example 30. 4-((2S,5R)-4-((4-Chloro-3-fluorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine

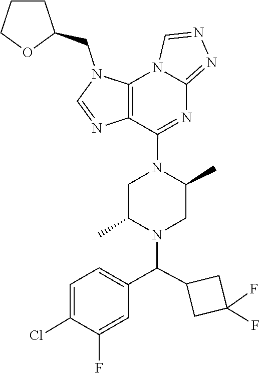

The title compound was prepared according to the procedures described in Example 1, with (2R,5S)-1-((4-chloro-3-fluorophenyl)(3,3-difluorocyclobutyl)methyl)-2,5-dimethylpiperazine hydrochloride (Intermediate 38) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₈H₃₃ClF₃N₈O (M+H)⁺. m/z=589.2. found 589.4. ¹H NMR (500 MHz, DMSO-d₆) (mixture of rotamers) δ 9.53 (s, 1H), 8.52-8.23 (m, 1H), 7.73-7.57 (m, 1H), 7.57-7.40 (m, 1H), 7.36-7.18 (m, 1H), 6.21-5.97 (m, 0.4H), 5.95-5.65 (m, 0.6H), 5.02-4.87 (m, 0.6H), 4.84-4.74 (m, 1H), 4.61-4.50 (m, 1.4H), 4.25-3.76 (m, 2H), 3.72-3.61 (m, 1.6H), 3.59-3.53 (m, 1H), 3.48-3.30 (m, 0.4H), 3.15-2.98 (m, 1H), 2.94-2.75 (m, 2H), 2.73-2.56 (m, 2H), 2.44-2.31 (m, 1H), 2.28-2.18 (m, 1H), 2.16-1.99 (m, 2H), 1.88-1.76 (m, 1H), 1.76-1.62 (m, 2H), 1.53-1.25 (m, 3H), 1.05-0.85 (m, 3H).

### Example 31. 4-((2S,5R)-4-((4-Chloro-2-fluorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine

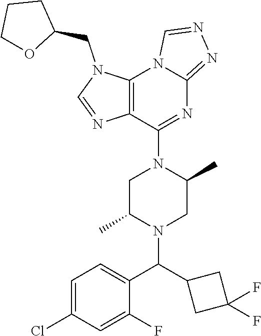 [SMILES: C[C@@H]1CN(c2nc3nncn3c3c2ncn3C[C@@H]2CCCO2)[C@@H](C)CN1C(c1ccc(Cl)cc1F)C1CC(F)(F)C1]

The title compound was prepared according to the procedures described in Example 1, with (2R,5S)-1-((4-chloro-2-fluorophenyl)(3,3-difluorocyclobutyl)methyl)-2,5-dimethylpiperazine hydrochloride (Intermediate 8) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₈H₃₃ClF₃N₈O (M+H)⁺. m/z=589.2. found 589.3. ¹H NMR (500 MHz, DMSO-d₆) (mixture of rotamers) δ 9.53 (s, 1H), 8.48-8.26 (m, 1H), 7.61-7.51 (m, 1H), 7.51-7.45 (m, 1H), 7.40-7.33 (m, 1H), 6.13-5.90 (m, 0.4H), 5.88-5.68 (m, 0.6H), 4.98-4.82 (m, 0.6H), 4.82-4.73 (m, 1H), 4.61-4.49 (m, 1.4H), 4.22-4.15 (m, 1H), 4.02-3.73 (m, 2H), 3.70-3.61 (m, 1.6H), 3.59-3.53 (m, 1H), 3.48-3.34 (m, 0.4H), 3.20-3.05 (m, 1H), 2.94-2.67 (m, 3H), 2.46-2.26 (m, 2H), 2.14-2.04 (m, 1H), 2.01-1.88 (m, 1H), 1.86-1.77 (m, 1H), 1.76-1.65 (m, 2H), 1.51-1.22 (m, 3H), 1.08-0.83 (m, 3H).

### Example 32. 4-((2S,5R)-4-((4-Chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine

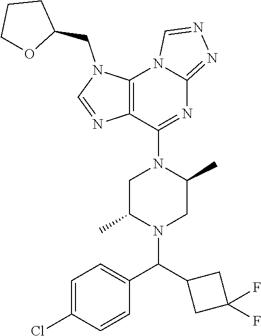 [SMILES: C[C@@H]1CN(c2nc3nncn3c3c2ncn3C[C@@H]2CCCO2)[C@@H](C)CN1C(c1ccc(Cl)cc1)C1CC(F)(F)C1]

The title compound was prepared according to the procedures described in Example 1, with (2R,5S)-1-((4-chlorophenyl)(3,3-difluorocyclobutyl)methyl)-2,5-dimethylpiperazine hydrochloride (Intermediate 39) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₈H₃₄ClF₂N₈O (M+H)⁺. m/z=571.3. found 571.4. ¹H NMR (500 MHz, DMSO-d₆) (mixture of rotamers) δ 9.54 (s, 1H), 8.48-8.24 (m, 1H), 7.66-7.28 (m, 4H), 6.18-5.99 (m, 0.4H), 5.96-5.57 (m, 0.6H), 5.04-4.88 (m, 0.6H), 4.88-4.74 (m, 1H), 4.65-4.47 (m, 1.4H), 4.24-4.08 (m, 2H), 3.71-3.61 (m, 1.6H), 3.61-3.51 (m, 1H), 3.45-3.27 (m, 0.4H), 3.22-3.00 (m, 1H), 2.97-2.74 (m, 2H), 2.74-2.54 (m, 2H), 2.47-2.32 (m, 1H), 2.29-2.16 (m, 1H), 2.14-1.96 (m, 2H), 1.89-1.77 (m, 1H), 1.77-1.66 (m, 2H), 1.54-1.27 (m, 3H), 1.09-0.84 (m, 3H).

### Example 33. 4-((2S,5R)-4-((4-Bromophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine

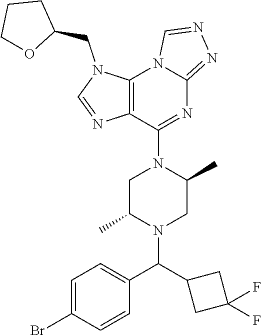 [SMILES: C[C@@H]1CN(c2nc3nncn3c3c2ncn3C[C@@H]2CCCO2)[C@@H](C)CN1C(c1ccc(Br)cc1)C1CC(F)(F)C1]

The title compound was prepared according to the procedures described in Example 1, with (2R,5S)-1-((4-bromophenyl)(3,3-difluorocyclobutyl)methyl)-2,5-dimethylpiperazine hydrochloride (Intermediate 40) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₈H₃₄BrF₂N₈O (M+H)⁺. m/z=615.2. found 615.3. ¹H NMR (500 MHz, DMSO-d₆) (mixture of rotamers) δ 9.54 (s, 1H), 8.51-8.26 (m, 1H), 7.67-7.49 (m, 2H), 7.47-7.30 (m, 2H), 6.21-6.00 (m, 0.4H), 5.94-5.70 (m, 0.6H), 5.02-4.90 (m, 0.6H), 4.87-4.75 (m, 1H), 4.65-4.46 (m, 1.4H), 4.28-4.09 (m, 1H), 3.82-3.60 (m, 2H), 3.60-3.49 (m, 1.6H), 3.47-3.34 (m, 0.4H), 3.25-3.05 (m, 1H), 3.01-2.54 (m, 4H), 2.47-2.31 (m, 1H), 2.29-2.14 (m, 1H), 2.13-1.92 (m, 2H), 1.85-1.76 (m, 1H), 1.76-1.60 (m, 2H), 1.53-1.27 (m, 3H), 1.11-0.82 (m, 3H).

### Example 34. 4-((2S,5R)-4-((4-Chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine

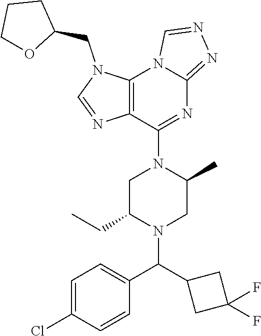 [SMILES: CC[C@@H]1CN(c2nc3nncn3c3c2ncn3C[C@@H]2CCCO2)[C@@H](C)CN1C(c1ccc(Cl)cc1)C1CC(F)(F)C1]

The title compound was prepared according to the procedures described in Example 1, with (2R,5S)-1-((4-chlorophenyl)(3,3-difluorocyclobutyl)methyl)-2-ethyl-5-methylpiperazine hydrochloride (Intermediate 43) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₉H₃₆ClF₂N₈O (M+H)⁺. m/z=585.3. found 585.3. ¹H NMR (500 MHz, DMSO-d₆) (mixture of rotamers) δ 9.54 (s, 1H), 8.46-8.31 (m, 1H), 7.57-7.44 (m, 2H), 7.44-7.34 (m, 2H), 6.01-5.86 (m, 1H), 4.87-4.75 (m, 1.6H), 4.65-4.53 (m, 1.4H), 4.25-4.15 (m, 1H), 3.85-3.71 (m, 1H), 3.69-3.61 (m, 1H), 3.60-3.46 (m, 1.6H), 3.37-3.21 (m, 0.4H), 3.20-2.99 (m, 1H), 2.86-2.55 (m, 4H), 2.45-2.32 (m, 1H), 2.31-2.15 (m, 1H), 2.14-2.05 (m, 1H), 2.05-1.93 (m, 1H), 1.88-1.76 (m, 1H), 1.75-1.63 (m, 2H), 1.60-1.50 (m, 1H), 1.48-1.40 (m, 1H), 1.39-1.23 (m, 3H), 0.88-0.72 (m, 3H).

### Example 35. 4-((2S,5R)-4-((3,3-Difluorocyclobutyl)(4-(trifluoromethyl)phenyl)methyl)-5-ethyl-2-methylpiperazin-1-yl)-1-(((S)-tetrahydrofuran-2-yl)methyl)-1H-[1,2,4]triazolo[3,4-b]purine

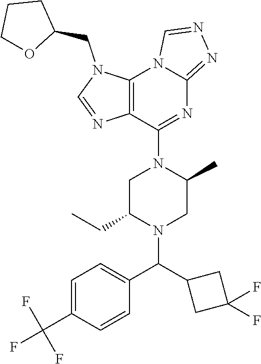 [SMILES: CC[C@@H]1CN(c2nc3nncn3c3c2ncn3C[C@@H]2CCCO2)[C@@H](C)CN1C(c1ccc(C(F)(F)F)cc1)C1CC(F)(F)C1]

The title compound was prepared according to the procedures described in Example 1, with (2R,5S)-1-((3,3-difluorocyclobutyl)(4-(trifluoromethyl)phenyl)methyl)-2-ethyl-5-methylpiperazine hydrochloride (Intermediate 44) replacing (2R,5S)-1-((3,3-difluorocyclobutyl)(4-(trifluoromethyl)phenyl)methyl)-2,5-dimethylpiperazine hydrochloride in Step 1. LC-MS calculated for C₃₀H₃₆F₅N₈O (M+H)⁺. m/z=619.3. found 619.4.

### Example 36. 4-((2S,5R)-5-Ethyl-2-methyl-4-(4-(trifluoromethyl)benzyl)piperazin-1-yl)-2-methyl-1-(((S)-tetrahydrofuran-2-yl)methyl)-1H-[1,2,4]triazolo[3,4-b]purine

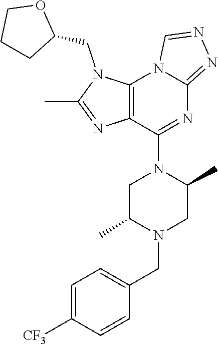 [SMILES: Cc1nc2c(N3C[C@@H](C)N(Cc4ccc(C(F)(F)F)cc4)C[C@@H]3C)nc3nncn3c2n1C[C@@H]1CCCO1]

### Step 1. tert-Butyl (2S,5R)-5-ethyl-2-methyl-4-(4-(trifluoromethyl)benzyl)piperazine-1-carboxylate

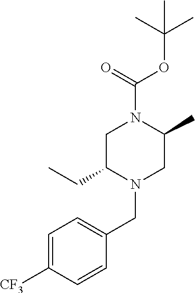 [SMILES: CC[C@@H]1CN(C(=O)OC(C)(C)C)[C@@H](C)CN1Cc1ccc(C(F)(F)F)cc1]

To a mixture of tert-butyl (2S,5R)-5-ethyl-2-methylpiperazine-1-carboxylate (Intermediate 41, 343 mg, 1.5 mmol) and 4-(trifluoromethyl)benzaldehyde (313 mg, 1.8 mmol) in CH₂Cl₂ (5.0 mL) was added AcOH (0.086 mL, 1.5 mmol), and the reaction mixture was allowed to stir at rt for 30 min, followed by addition of sodium triacetoxyhydroborate (477 mg, 2.25 mmol). The mixture was further allowed to stir at rt for 4 h. The reaction mixture was diluted with EtOAc and water. The layers were separated and the aqueous layer was extracted with EtOAc. The combined organic layers were washed with brine, dried over MgSO₄, concentrated in vacuo, and purified by flash column chromatography (4 g SiO₂, EtOAc/hexanes) to give the desired product as a white solid. LC-MS calculated for C₂₀H₃₀F₃N₂O₂(M+H)⁺: m/z=387.2. found 387.3.

### Step 2. (2R,5S)-2-Ethyl-5-methyl-1-(4-(trifluoromethyl)benzyl)piperazine Hydrochloride

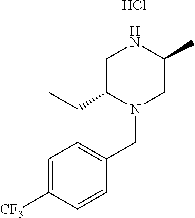 [SMILES: Cl ; CC[C@@H]1CN[C@@H](C)CN1Cc1ccc(C(F)(F)F)cc1]

To a mixture of tert-butyl (2S,5R)-5-ethyl-2-methyl-4-(4-(trifluoromethyl)benzyl)piperazine-1-carboxylate (Step 1) in CH₂Cl₂ (5 mL) was added a 4 molar solution of HCl in 1,4-dioxane (0.5 mL, 2 mmol), and the reaction mixture was allowed to stir at rt for 4 h. The reaction mixture was concentrated in vacuo, and the crude material obtained was used directly without further purification. LC-MS calculated for C₁₅H₂₂F₃N₂ (M+H)⁺: m/z=287.2. found 287.2.

### Step 3. 2-Chloro-6-((2S,5R)-5-ethyl-2-methyl-4-(4-(trifluoromethyl)benzyl)piperazin-1-yl)-8-methyl-9-(((S)-tetrahydrofuran-2-yl)methyl)-9H-purine

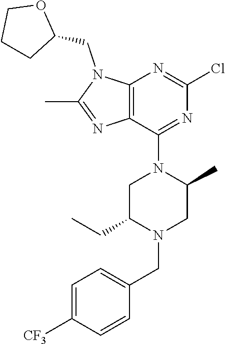 [SMILES: CC[C@@H]1CN(c2nc(Cl)nc3c2nc(C)n3C[C@@H]2CCCO2)[C@@H](C)CN1Cc1ccc(C(F)(F)F)cc1]

To a mixture of (S)-2,6-dichloro-8-methyl-9-((tetrahydrofuran-2-yl)methyl)-9H-purine (Intermediate 5, 517 mg, 1.8 mmol) and (2R,5S)-2-ethyl-5-methyl-1-(4-(trifluoromethyl)benzyl)piperazine hydrochloride (Step 2) in MeCN (5.0 mL) was added potassium carbonate (415 mg, 3.0 mmol) and the reaction mixture was stirred at 90° C. overnight. After cooling to rt, the reaction mixture was filtered over Celite and the filtrate was concentrated in vacuo. The crude residue was purified directly by flash column chromatography (12 g SiO₂, EtOAc/hexanes) to afford the desired product as a light yellow foam. LC-MS calculated for C₂₆H₃₃ClF₃N₆O (M+H)⁺: m/z=537.2. found 537.3.

### Step 4. 6-((2S,5R)-5-Ethyl-2-methyl-4-(4-(trifluoromethyl)benzyl)piperazin-1-yl)-2-hydrazineyl-8-methyl-9-(((S)-tetrahydrofuran-2-yl)methyl)-9H-purine

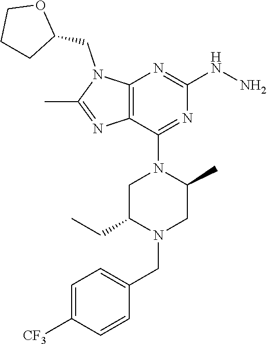 [SMILES: CC[C@@H]1CN(c2nc(NN)nc3c2nc(C)n3C[C@@H]2CCCO2)[C@@H](C)CN1Cc1ccc(C(F)(F)F)cc1]

To a mixture of 2-chloro-6-((2S,5R)-5-ethyl-2-methyl-4-(4-(trifluoromethyl)benzyl)piperazin-1-yl)-8-methyl-9-(((S)-tetrahydrofuran-2-yl)methyl)-9H-purine (Step 3), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (128 mg, 0.15 mmol), and cesium carbonate (2.4 g, 7.5 mmol) was added a 1 molar solution of hydrazine in THF (7.5 mL, 7.5 mmol) and the mixture was stirred at 60° C. for 30 min. After cooling to rt, the reaction mixture was filtered through a pad of MgSO₄ in a SiliaPrep SPE thiol cartridge (SiliCycle SPE-R51030B-06P). The filtrate was concentrated, and the crude material obtained was used directly without further purification. LC-MS calculated for C₂₆H₃₆F₃N₈O (M+H)⁺: m/z=533.3. found 533.3.

### Step 5. 4-((2S,5R)-5-Ethyl-2-methyl-4-(4-(trifluoromethyl)benzyl)piperazin-1-yl)-2-methyl-1-(((S)-tetrahydrofuran-2-yl)methyl)-1H-[1,2,4]triazolo[3,4-b]purine

A mixture of 6-((2S,5R)-5-ethyl-2-methyl-4-(4-(trifluoromethyl)benzyl)piperazin-1-yl)-2-hydrazineyl-8-methyl-9-(((S)-tetrahydrofuran-2-yl)methyl)-9H-purine (Step 4), triethyl orthoformate (2 mL, 18.1 mmol), and AcOH (0.2 mL, 3.5 mmol) was stirred at 90° C. for 1 h. After cooling to rt, the reaction mixture was diluted with acetonitrile and water and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₇H₃₄F₃N₈O (M+H)⁺: m/z=543.3. found 543.3. ¹H NMR (600 MHz, DMSO-d₆, 70° C.) (mixture of rotamers) δ9.46-9.43 (m, 1H), 7.76-7.69 (m, 2H), 7.69-7.64 (m, 2H), 6.48-5.16 (m, 2H), 4.77-4.70 (m, 1H), 4.62-4.53 (m, 1H), 4.19-4.10 (m, 1H), 3.94-3.88 (m, 2H), 3.76-3.68 (m, 2H), 3.66-3.54 (m, 1H), 3.06-2.98 (m, 1H), 2.96-2.87 (m, 1H), 2.77-2.56 (m, 4H), 2.21-2.11 (m, 1H), 1.99-1.80 (m, 2H), 1.78-1.67 (m, 1H), 1.69-1.58 (m, 1H), 1.54-1.44 (m, 1H), 1.44-1.39 (m, 3H), 0.99-0.92 (m, 3H).

### Example 37. 4-((2S,5R)-4-(2-Fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine

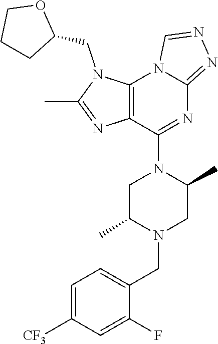 [SMILES: Cc1nc2c(N3C[C@@H](C)N(Cc4ccc(C(F)(F)F)cc4F)C[C@@H]3C)nc3nncn3c2n1C[C@@H]1CCCO1]

To a mixture of 4-((2S,5R)-2,5-dimethylpiperazin-1-yl)-2-methyl-1-(((S)-tetrahydrofuran-2-yl)methyl)-1H-[1,2,4]triazolo[3,4-b]purine hydrochloride (Intermediate 45, 10 mg, 0.025 mmol) in CH₂Cl₂ (0.5 mL) was added N,N-diisopropylethylamine (8.6 μL, 0.049 mmol) and 2-fluoro-4-(trifluoromethyl)benzaldehyde (9.4 mg, 0.049 mmol) and the reaction mixture was stirred at rt for 30 min. AcOH (2.1 μL, 0.037 mmol) was added and the reaction mixture was stirred at rt for 10 min before sodium triacetoxyborohydride (10.4 mg, 0.049 mmol) was added and the reaction mixture was stirred at rt for 4 h. The reaction mixture was diluted with methanol and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₆H₃₁F₄N₈O (M+H)⁺. m/z=547.3. found 547.3. ¹H NMR (600 MHz, DMSO-d₆, 70° C.) (mixture of rotamers) δ 9.45-9.42 (m, 1H), 7.84-7.78 (m, 1H), 7.64-7.59 (m, 2H), 5.83-5.23 (m, 2H), 4.77-4.70 (m, 1H), 4.61-4.52 (m, 1H), 4.17-4.10 (m, 1H), 3.87-3.81 (m, 1H), 3.76-3.68 (m, 3H), 3.62-3.55 (m, 1H), 3.34-3.22 (m, 1H), 3.02-2.97 (m, 1H), 2.67-2.60 (m, 3H), 2.58-2.52 (m, 1H), 2.23-2.11 (m, 1H), 1.97-1.80 (m, 2H), 1.78-1.67 (m, 1H), 1.44-1.39 (m, 3H), 1.09-1.05 (m, 3H).

### Example 38. 4-((2S,5R)-4-(2-Fluoro-4-(trifluoromethyl)benzyl)-2,5-dimethylpiperazin-1-yl)-1-(((S)-tetrahydrofuran-2-yl)methyl)-1H-[1,2,4]triazolo[3,4-b]purine

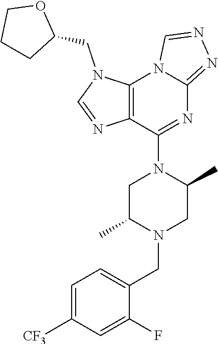 [SMILES: C[C@@H]1CN(c2nc3nncn3c3c2ncn3C[C@@H]2CCCO2)[C@@H](C)CN1Cc1ccc(C(F)(F)F)cc1F]

To a mixture of 4-((2S,5R)-2,5-dimethylpiperazin-1-yl)-1-(((S)-tetrahydrofuran-2-yl)methyl)-1H-[1,2,4]triazolo[3,4-b]purine hydrochloride (Intermediate 46, 20 mg, 0.051 mmol) in CH₂Cl₂ (0.5 mL) was added N,N-diisopropylethylamine (18 μL, 0.10 mmol) and 2-fluoro-4-(trifluoromethyl)benzaldehyde (14.7 mg, 0.076 mmol) and the reaction mixture was stirred at rt for 30 min. AcOH (4.4 μL, 0.076 mmol) was added and the reaction mixture was stirred at rt for 10 min before sodium triacetoxyborohydride (16.2 mg, 0.076 mmol) was added and the reaction mixture was stirred at rt for 4 h. The reaction mixture was diluted with methanol and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₅H₂₉F₄N₈O (M+H)⁺. m/z=533.2. found 533.2. ¹H NMR (600 MHz, DMSO-d₆) (mixture of rotamers) δ9.61-9.46 (m, 1H), 8.49-8.23 (m, 1H), 7.91-7.80 (m, 1H), 7.76-7.57 (m, 2H), 6.28-5.77 (m, 1H), 5.36-4.66 (m, 2H), 4.71-4.48 (m, 1H), 4.32-4.13 (m, 1H), 4.00-3.73 (m, 2H), 3.71-3.61 (m, 1H), 3.61-3.52 (m, 1H), 3.48-2.77 (m, 3H), 2.47-2.27 (m, 1H), 2.20-1.97 (m, 1H), 1.89-1.67 (m, 3H), 1.52-1.30 (m, 3H), 1.24-0.99 (m, 3H).

### Example 39. 4-((2S,5R)-2,5-Dimethyl-4-(4-(trifluoromethyl)benzyl)piperazin-1-yl)-2-methyl-1-(((S)-tetrahydrofuran-2-yl)methyl)-1H-[1,2,4]triazolo[3,4-b]purine

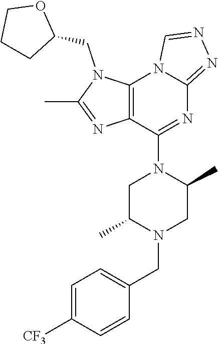 [SMILES: Cc1nc2c(N3C[C@@H](C)N(Cc4ccc(C(F)(F)F)cc4)C[C@@H]3C)nc3nncn3c2n1C[C@@H]1CCCO1]

The title compound was prepared according to the procedures described in Example 37, with 4-(trifluoromethyl)benzaldehyde replacing 2-fluoro-4-(trifluoromethyl)benzaldehyde. LC-MS calculated for C₂₆H₃₂F₃N₈O (M+H)⁺. m/z=529.3. found 529.3. ¹H NMR (600 MHz, DMSO-d₆) (mixture of rotamers) δ9.61-9.22 (m, 1H), 7.94-7.60 (m, 4H), 6.52-5.85 (m, 1H), 5.48-4.99 (m, 1H), 4.86-4.68 (m, 1H), 4.66-4.49 (m, 1H), 4.25-4.07 (m, 1H), 4.05-3.75 (m, 3H), 3.74-3.64 (m, 1H), 3.62-3.55 (m, 1H), 3.51-2.78 (m, 2H), 2.71-2.60 (m, 4H), 2.29-2.08 (m, 1H), 2.03-1.91 (m, 1H), 1.89-1.79 (m, 1H), 1.78-1.68 (m, 1H), 1.51-1.33 (m, 3H), 1.33-0.90 (m, 3H).

### Example 40. 4-((2S,5R)-2,5-Dimethyl-4-(4-(trifluoromethyl)benzyl)piperazin-1-yl)-1-(((S)-tetrahydrofuran-2-yl)methyl)-1H-[1,2,4]triazolo[3,4-b]purine

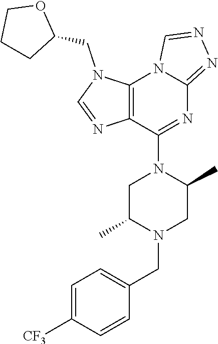 [SMILES: C[C@@H]1CN(c2nc3nncn3c3c2ncn3C[C@@H]2CCCO2)[C@@H](C)CN1Cc1ccc(C(F)(F)F)cc1]

The title compound was prepared according to the procedures described in Example 38, with 4-(trifluoromethyl)benzaldehyde replacing 2-fluoro-4-(trifluoromethyl)benzaldehyde. LC-MS calculated for C₂₅H₃₀F₃N₈O (M+H)⁺: m/z=515.2. found 515.2. ¹H NMR (600 MHz, DMSO-d₆) (mixture of rotamers) δ9.80-9.13 (m, 1H), 8.98-8.32 (m, 1H), 7.88-7.65 (m, 4H), 6.50-5.85 (m, 1H), 5.55-4.98 (m, 1H), 4.93-4.80 (m, 1H), 4.67-4.55 (m, 1H), 4.24-4.13 (m, 1H), 4.08-3.75 (m, 3H), 3.74-3.62 (m, 1H), 3.61-3.50 (m, 1H), 3.44-2.82 (m, 2H), 2.76-2.58 (m, 1H), 2.16-2.02 (m, 1H), 1.93-1.63 (m, 3H), 1.52-1.33 (m, 3H), 1.28-0.97 (m, 3H).

### Examples 41 and 42. 4-((2S,5R)-5-Ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine and 4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine

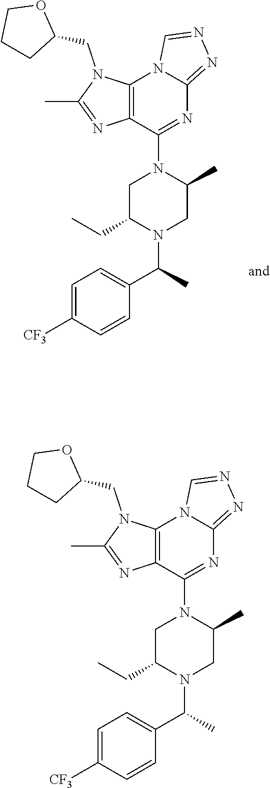 [SMILES: CC[C@@H]1CN(c2nc3nncn3c3c2nc(C)n3C[C@@H]2CCCO2)[C@@H](C)CN1[C@@H](C)c1ccc(C(F)(F)F)cc1 ; CC[C@@H]1CN(c2nc3nncn3c3c2nc(C)n3C[C@@H]2CCCO2)[C@@H](C)CN1[C@H](C)c1ccc(C(F)(F)F)cc1]

### Step 1. 1-(4-(Trifluoromethyl)phenyl)ethyl Methanesulfonate

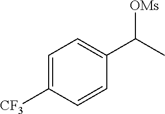 [SMILES: CC(OS(C)(=O)=O)c1ccc(C(F)(F)F)cc1]

To a mixture of 1-(4-(trifluoromethyl)phenyl)ethan-1-ol (0.20 mL, 1.3 mmol) and methanesulfonyl chloride (0.11 mL, 1.43 mmol) in CH₂Cl₂ (5.0 mL) was added triethylamine (0.2 mL, 1.43 mmol), and the reaction mixture was allowed to stir at rt overnight. The reaction mixture was concentrated in vacuo and used directly for next step without further purification.

### Step 2. tert-Butyl (2S,5R)-5-ethyl-2-methyl-4-(1-(4-(trifluoromethyl)phenyl)ethyl)piperazine-1-carboxylate

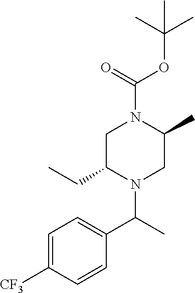 [SMILES: CC[C@@H]1CN(C(=O)OC(C)(C)C)[C@@H](C)CN1C(C)c1ccc(C(F)(F)F)cc1]

To a mixture of tert-butyl (2S,5R)-5-ethyl-2-methylpiperazine-1-carboxylate (Intermediate 41, 228 mg, 1.0 mmol) and 1-(4-(trifluoromethyl)phenyl)ethyl methanesulfonate (Step 1) in MeCN (5.0 mL) was added N,N-diisopropylethylamine (0.35 mL, 2.0 mmol), and the reaction mixture was allowed to stir at 90° C. overnight. The reaction mixture was diluted with EtOAc and water. The layers were separated and the aqueous layer was extracted with EtOAc. The combined organic layers were washed with brine, dried over MgSO₄, concentrated in vacuo, and purified by flash column chromatography (4 g SiO₂, EtOAc/hexanes) to give the desired product as a yellow solid. LC-MS calculated for C₂₁H₃₂F₃N₂O₂(M+H)⁺. m/z=401.2. found 401.2.

### Step 3. (2R,5S)-2-Ethyl-5-methyl-1-(1-(4-(trifluoromethyl)phenyl)ethyl)piperazine Hydrochloride

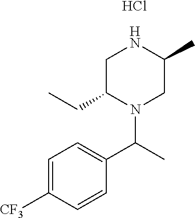 [SMILES: Cl ; CC[C@@H]1CN[C@@H](C)CN1C(C)c1ccc(C(F)(F)F)cc1]

To a mixture of tert-butyl (2S,5R)-5-ethyl-2-methyl-4-(1-(4-(trifluoromethyl)phenyl)ethyl)piperazine-1-carboxylate (Step 2) in CH₂Cl₂ (1.0 mL) was added a 4 molar solution of HCl in 1,4-dioxane (0.5 mL, 2 mmol), and the reaction mixture was allowed to stir at rt for 4 h. The reaction mixture was concentrated in vacuo, and the crude material obtained was used directly without further purification. LC-MS calculated for C₁₆H₂₄F₃N₂ (M+H)⁺: m/z=301.2. found 301.2.

### Step 4. 2-Chloro-6-((2S,5R)-5-ethyl-2-methyl-4-(1-(4-(trifluoromethyl)phenyl)ethyl)piperazin-1-yl)-8-methyl-9-(((S)-tetrahydrofuran-2-yl)methyl)-9H-purine

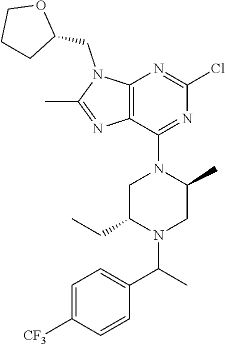 [SMILES: CC[C@@H]1CN(c2nc(Cl)nc3c2nc(C)n3C[C@@H]2CCCO2)[C@@H](C)CN1C(C)c1ccc(C(F)(F)F)cc1]

To a mixture of (S)-2,6-dichloro-8-methyl-9-((tetrahydrofuran-2-yl)methyl)-9H-purine (Intermediate 5, 344 mg, 1.2 mmol) and (2R,5S)-2-ethyl-5-methyl-1-(1-(4-(trifluoromethyl)phenyl)ethyl)piperazine hydrochloride (Step 3) in MeCN (5.0 mL) was added potassium carbonate (276 mg, 2.0 mmol) and the reaction mixture was stirred at 90° C. overnight. After cooling to rt, the reaction mixture was filtered over Celite and the filtrate was concentrated in vacuo. The crude residue was used directly for next step without further purification. LC-MS calculated for C₂₇H₃₅ClF₃N₆O (M+H)⁺: m/z=551.2. found 551.3.

### Step 5. 6-((2S,5R)-5-Ethyl-2-methyl-4-(1-(4-(trifluoromethyl)phenyl)ethyl)piperazin-1-yl)-2-hydrazineyl-8-methyl-9-(((S)-tetrahydrofuran-2-yl)methyl)-9H-purine

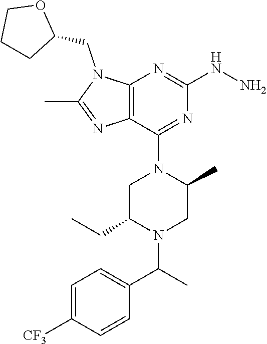 [SMILES: CC[C@@H]1CN(c2nc(NN)nc3c2nc(C)n3C[C@@H]2CCCO2)[C@@H](C)CN1C(C)c1ccc(C(F)(F)F)cc1]

To a mixture of 2-chloro-6-((2S,5R)-5-ethyl-2-methyl-4-(1-(4-(trifluoromethyl)phenyl)ethyl)piperazin-1-yl)-8-methyl-9-(((S)-tetrahydrofuran-2-yl)methyl)-9H-purine (Step 4), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (85 mg, 0.10 mmol), and cesium carbonate (1.63 g, 5.0 mmol) was added a 1 molar solution of hydrazine in THF (5.0 mL, 5.0 mmol) and the mixture was stirred at 90° C. for 30 min. After cooling to rt, the reaction mixture was filtered through a pad of MgSO₄ in a SiliaPrep SPE thiol cartridge (SiliCycle SPE-R51030B-06P). The filtrate was concentrated, and the crude material obtained was used directly without further purification. LC-MS calculated for C₂₇H₃₈F₃N₈O (M+H)⁺. m/z=547.3. found 547.3.

### Step 6. 4-((2S,5R)-5-Ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine and 4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine

A mixture of 6-((2S,5R)-5-ethyl-2-methyl-4-(1-(4-(trifluoromethyl)phenyl)ethyl)piperazin-1-yl)-2-hydrazineyl-8-methyl-9-(((S)-tetrahydrofuran-2-yl)methyl)-9H-purine (Step 5), triethyl orthoformate (1.0 mL, 6.0 mmol), and AcOH (0.034 mL, 0.60 mmol) was stirred at 90° C. for 1 h. After cooling to rt, the reaction mixture was diluted with acetonitrile and water and purified by prep-HPLC (Sunfire C18 column, eluting with a gradient of acetonitrile/water containing 0.1% TFA, at flow rate of 60 mL/min) to afford each diastereomer as its TFA salt.

Example 41: Retention time on LC-MS tᵣ=1.42 min. LC-MS calculated for C₂₈H₃₆F₃N₈O (M+H)⁺: m/z=557.3. found 557.3. ¹H NMR (600 MHz, DMSO-d₆, 70° C.) (mixture of rotamers) δ9.44-9.41 (m, 1H), 7.77-7.72 (m, 2H), 7.69-7.64 (m, 2H), 6.34-5.77 (m, 2H), 4.76-4.69 (m, 1H), 4.60-4.52 (m, 1H), 4.18-4.09 (m, 1H), 4.07-3.95 (m, 1H), 3.75-3.67 (m, 1H), 3.66-3.42 (m, 2H), 3.11-2.84 (m, 2H), 2.72-2.54 (m, 4H), 2.20-2.10 (m, 1H), 2.00-1.89 (m, 1H), 1.89-1.81 (m, 1H), 1.77-1.67 (m, 1H), 1.59-1.33 (m, 8H), 0.79-0.73 (m, 3H).

Example 42: Retention time on LC-MS tᵣ=1.45 min. LC-MS calculated for C₂₈H₃₆F₃N₈O (M+H)⁺: m/z=557.3. found 557.3. ¹H NMR (600 MHz, DMSO-d₆, 70° C.) (mixture of rotamers) δ9.45-9.41 (m, 1H), 7.77-7.64 (m, 4H), 6.35-5.94 (m, 1H), 5.19-4.85 (m, 1H), 4.76-4.69 (m, 1H), 4.61-4.52 (m, 1H), 4.18-4.10 (m, 1H), 3.98-3.68 (m, 3H), 3.62-3.54 (m, 1H), 3.39-3.19 (m, 1H), 2.88-2.70 (m, 1H), 2.68-2.56 (m, 3H), 2.41-2.28 (m, 1H), 2.20-2.10 (m, 1H), 1.98-1.80 (m, 2H), 1.80-1.67 (m, 1H), 1.60-1.43 (m, 2H), 1.39-1.31 (m, 6H), 1.04-0.97 (m, 3H).

### Example 43. 4-((2S,5R)-4-(Bis(4-fluorophenyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

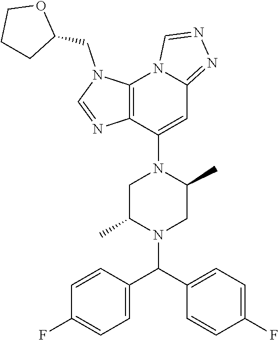 [SMILES: C[C@@H]1CN(c2cc3nncn3c3c2ncn3C[C@@H]2CCCO2)[C@@H](C)CN1C(c1ccc(F)cc1)c1ccc(F)cc1]

### Step 1. 4-((2S,5R)-4-(Bis(4-fluorophenyl)methyl)-2,5-dimethylpiperazin-1-yl)-6-chloro-3-nitropyridin-2-amine

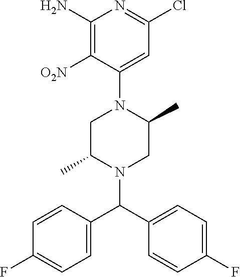 [SMILES: C[C@@H]1CN(c2cc(Cl)nc(N)c2[N+](=O)[O-])[C@@H](C)CN1C(c1ccc(F)cc1)c1ccc(F)cc1]

A mixture of (2R,5S)-1-(bis(4-fluorophenyl)methyl)-2,5-dimethylpiperazine hydrochloride (Intermediate 49, 309 mg, 0.79 mmol) and 4,6-dichloro-3-nitropyridin-2-amine (150 mg, 0.72 mmol, ChemScene CS-0094679) in MeCN (3.0 mL) was added potassium carbonate (299 mg, 2.16 mmol), and the reaction mixture was stirred at 90° C. overnight. After cooling to rt, the reaction mixture was filtered over Celite and the filtrate was concentrated in vacuo. The crude residue was used directly for next step without further purification. LC-MS calculated for C₂₄H₂₅ClF₂N₅O₂ (M+H)⁺. m/z=488.2. found 488.2.

### Step 2. 4-((2S,5R)-4-(Bis(4-fluorophenyl)methyl)-2,5-dimethylpiperazin-1-yl)-6-chloropyridine-2,3-diamine

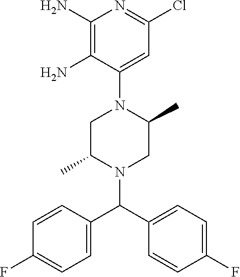 [SMILES: C[C@@H]1CN(c2cc(Cl)nc(N)c2N)[C@@H](C)CN1C(c1ccc(F)cc1)c1ccc(F)cc1]

A mixture of 4-((2S,5R)-4-(bis(4-fluorophenyl)methyl)-2,5-dimethylpiperazin-1-yl)-6-chloro-3-nitropyridin-2-amine (Step 1) in DMF (2.0 mL) was cooled to 0° C. in an ice-bath before hypodiboric acid (0.194 g, 2.16 mmol) was added, followed by dropwise addition of a solution of 4,4′-dipyridyl (1.1 mg, 7.2 μmol) in DMF (0.5 mL). The mixture was stirred at 0° C. for 5 min, at which point the mixture was diluted with water and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₄H₂₇ClF₂N₅ (M+H)⁺: m/z=458.2. found 458.2.

### Step 3. 7-((2S,5R)-4-(Bis(4-fluorophenyl)methyl)-2,5-dimethylpiperazin-1-yl)-5-chloro-3H-imidazo[4,5-b]pyridine

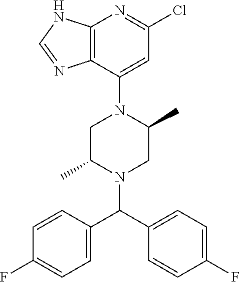 [SMILES: C[C@@H]1CN(c2cc(Cl)nc3[nH]cnc23)[C@@H](C)CN1C(c1ccc(F)cc1)c1ccc(F)cc1]

A mixture of 4-((2S,5R)-4-(bis(4-fluorophenyl)methyl)-2,5-dimethylpiperazin-1-yl)-6-chloropyridine-2,3-diamine (Step 2) and acetic acid (0.50 mL, 0.35 mmol) in triethyl orthoformate (0.5 mL, 3.0 mmol) was stirred at 120° C. for 4 h. The mixture was diluted with saturated aqueous NaHCO₃ and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₅H₂₅ClF₂N₅ (M+H)⁺: m/z=468.2. found 468.2.

### Step 4. 7-((2S,5R)-4-(Bis(4-fluorophenyl)methyl)-2,5-dimethylpiperazin-1-yl)-5-chloro-3-(((S)-tetrahydrofuran-2-yl)methyl)-3H-imidazo[4,5-b]pyridine

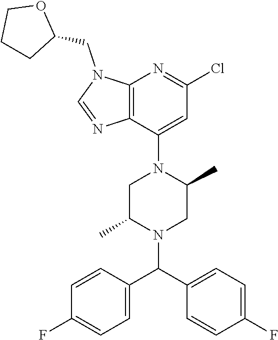 [SMILES: C[C@@H]1CN(c2cc(Cl)nc3c2ncn3C[C@@H]2CCCO2)[C@@H](C)CN1C(c1ccc(F)cc1)c1ccc(F)cc1]

A mixture of 7-((2S,5R)-4-(bis(4-fluorophenyl)methyl)-2,5-dimethylpiperazin-1-yl)-5-chloro-3H-imidazo[4,5-b]pyridine (Step 3) in MeCN (1.0 mL) was added cesium carbonate (171 mg, 0.52 mmol) and (S)-(tetrahydrofuran-2-yl)methyl methanesulfonate (Intermediate 50, 95 mg, 0.52 mmol) and the reaction mixture was stirred at 90° C. overnight. After cooling to rt, the reaction mixture was filtered through a pad of Celite and concentrated in vacuo. The crude residue was purified by flash column chromatography (12 g SiO₂, EtOAc/hexanes) to afford the desired product as a white solid. LC-MS calculated for C₃₀H₃₃ClF₂N₅O (M+H)⁺. m/z=552.2. found 552.3.

### Step 5. 7-((2S,5R)-4-(Bis(4-fluorophenyl)methyl)-2,5-dimethylpiperazin-1-yl)-5-hydrazineyl-3-(((S)-tetrahydrofuran-2-yl)methyl)-3H-imidazo[4,5-b]pyridine

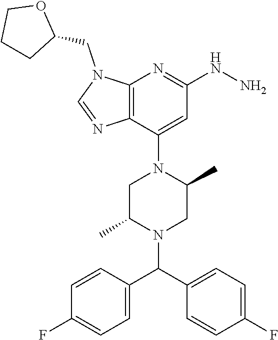 [SMILES: C[C@@H]1CN(c2cc(NN)nc3c2ncn3C[C@@H]2CCCO2)[C@@H](C)CN1C(c1ccc(F)cc1)c1ccc(F)cc1]

To a mixture of 7-((2S,5R)-4-(bis(4-fluorophenyl)methyl)-2,5-dimethylpiperazin-1-yl)-5-chloro-3-(((S)-tetrahydrofuran-2-yl)methyl)-3H-imidazo[4,5-b]pyridine (50 mg, 0.09 mmol), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (7.7 mg, 0.009 mmol), and cesium carbonate (148 mg, 0.45 mmol) was added a 1 molar solution of hydrazine in THF (0.45 mL, 0.45 mmol) and the mixture was stirred at 90° C. for 30 min. After cooling to rt, the reaction mixture was filtered through a pad of MgSO₄ in a SiliaPrep SPE thiol cartridge (SiliCycle SPE-R51030B-06P). The filtrate was concentrated, and the crude material obtained was used directly without further purification. LC-MS calculated for C₃₀H₃₆F₂N₇O (M+H)⁺: m/z=548.3. found 548.3.

### Step 6. 4-((2S,5R)-4-(Bis(4-fluorophenyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

A mixture of 7-((2S,5R)-4-(bis(4-fluorophenyl)methyl)-2,5-dimethylpiperazin-1-yl)-5-hydrazineyl-3-(((S)-tetrahydrofuran-2-yl)methyl)-3H-imidazo[4,5-b]pyridine (Step 5), triethyl orthoformate (0.50 mL, 3.0 mmol), and AcOH (0.025 mL, 0.44 mmol) was stirred at 90° C. for 1 h. After cooling to rt, the reaction mixture was diluted with acetonitrile and water and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₁H₃₄F₂N₇O (M+H)⁺: m/z=558.3. found 558.3. ¹H NMR (600 MHz, DMSO-d₆) (mixture of rotamers) δ9.77-9.14 (m, 1H), 8.57-7.96 (m, 1H), 7.80-7.49 (m, 4H), 7.35-7.08 (m, 4H), 6.74-6.28 (m, 1H), 4.88-4.77 (m, 1H), 4.71-4.64 (m, 1H), 4.64-4.50 (m, 1H), 4.26-4.07 (m, 1H), 3.71-3.62 (m, 1H), 3.62-3.57 (m, 1H), 3.56-3.51 (m, 1H), 3.40-3.33 (m, 1H), 3.18-3.09 (m, 1H), 3.02-2.91 (m, 1H), 2.86-2.76 (m, 1H), 2.43-2.33 (m, 1H), 2.13-2.02 (m, 1H), 1.86-1.65 (m, 3H), 1.42-1.36 (m, 3H), 1.00-0.95 (m, 3H).

### Example 44. 1-((4-((2S,5R)-4-(Bis(4-chlorophenyl)methyl)-2,5-dimethylpiperazin-1-yl)-1H-[1,2,4]triazolo[3,4-b]purin-1-yl)methyl)cyclopentan-1-ol

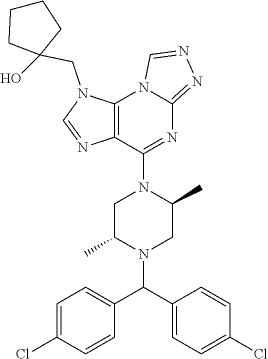 [SMILES: C[C@@H]1CN(c2nc3nncn3c3c2ncn3CC2(O)CCCC2)[C@@H](C)CN1C(c1ccc(Cl)cc1)c1ccc(Cl)cc1]

### Step 1: 1-(((6-((2S,5R)-4-(Bis(4-chlorophenyl)methyl)-2,5-dimethylpiperazin-1-yl)-2-chloro-5-nitropyrimidin-4-yl)amino)methyl)cyclopentan-1-ol

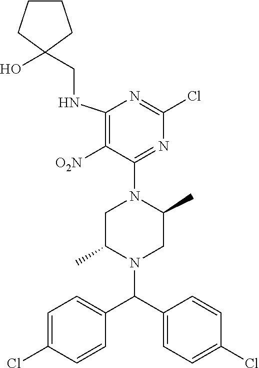 [SMILES: C[C@@H]1CN(c2nc(Cl)nc(NCC3(O)CCCC3)c2[N+](=O)[O-])[C@@H](C)CN1C(c1ccc(Cl)cc1)c1ccc(Cl)cc1]

A mixture of 2,4,6-trichloro-5-nitropyrimidine (200 mg, 0.876 mmol, Combi-Blocks, ST-3909) and (2R,5S)-1-(bis(4-chlorophenyl)methyl)-2,5-dimethylpiperazine hydrochloride (Intermediate 51, 370 mg, 0.959 mmol) in CH₂Cl₂ (10 mL) was cooled to 0° C. in an ice-bath before N-ethyl-N-isopropylpropan-2-amine (0.612 μL, 3.50 mmol) was added and the reaction mixture was stirred at 0° C. for 30 min. To the mixture was added 1-(aminomethyl)cyclopentan-1-ol hydrochloride (146 mg, 0.963 mmol) and the reaction mixture was warmed to rt and stirred for 30 min. The mixture was diluted with saturated aqueous NaHCO₃ and extracted with EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₉H₃₄Cl₃N₆O₃ (M+H)⁺: m/z=619.2. found 619.2.

### Step 2. 1-(((5-Amino-6-((2S,5R)-4-(bis(4-chlorophenyl)methyl)-2,5-dimethylpiperazin-1-yl)-2-chloropyrimidin-4-yl)amino)methyl)cyclopentan-1-ol

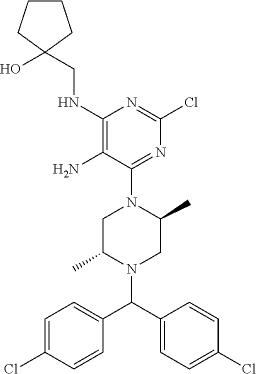 [SMILES: C[C@@H]1CN(c2nc(Cl)nc(NCC3(O)CCCC3)c2N)[C@@H](C)CN1C(c1ccc(Cl)cc1)c1ccc(Cl)cc1]

To a mixture of 1-(((6-((2S,5R)-4-(bis(4-chlorophenyl)methyl)-2,5-dimethylpiperazin-1-yl)-2-chloro-5-nitropyrimidin-4-yl)amino)methyl)cyclopentan-1-ol (Step 1) in CH₃CN (20 mL) and MeOH (5 mL) was added tetrahydroxydiboron (235 mg, 2.63 mmol) followed by N-ethyl-N-isopropylpropan-2-amine (0.612 mL, 3.50 mmol) and 4,4′-dipyridyl (13.7 mg, 0.088 mmol) and the reaction mixture was stirred at rt for 10 min. The mixture was diluted with saturated aqueous NaHCO₃ and extracted with EtOAc. The organic layer was removed, and the aqueous layer was extracted with EtOAc. The combined organic layers were dried over MgSO₄, filtered, and concentrated. The crude residue was purified by flash column chromatography (SiO₂, 0-5% MeOH/CH₂Cl₂) to afford the desired product (0.45 g, 87% yield over 2 steps). LC-MS calculated for C₂₉H₃₆Cl₃N₆O (M+H)⁺: m/z=589.2. found 589.3.

### Step 3. 1-((6-((2S,5R)-4-(Bis(4-chlorophenyl)methyl)-2,5-dimethylpiperazin-1-yl)-2-chloro-9H-purin-9-yl)methyl)cyclopentan-1-ol

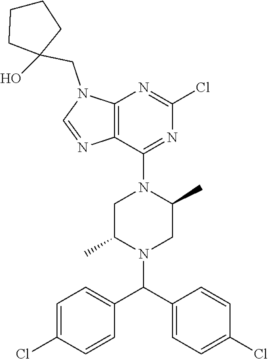 [SMILES: C[C@@H]1CN(c2nc(Cl)nc3c2ncn3CC2(O)CCCC2)[C@@H](C)CN1C(c1ccc(Cl)cc1)c1ccc(Cl)cc1]

To a mixture of 1-(((5-amino-6-((2S,5R)-4-(bis(4-chlorophenyl)methyl)-2,5-dimethylpiperazin-1-yl)-2-chloropyrimidin-4-yl)amino)methyl)cyclopentan-1-ol (Step 2) and triethyl orthoformate (520 mg, 3.5 mmol) in AcOH (4 mL) was stirred at 90° C. for 2 h. After cooling to rt, the mixture was diluted with saturated aqueous NaHCO₃ and extracted with EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₃₀H₃₄Cl₃N₆O (M+H)⁺. m/z=599.2. found 599.2.

### Step 4. 1-((6-((2S,5R)-4-(Bis(4-chlorophenyl)methyl)-2,5-dimethylpiperazin-1-yl)-2-hydrazineyl-9H-purin-9-yl)methyl)cyclopentan-1-ol

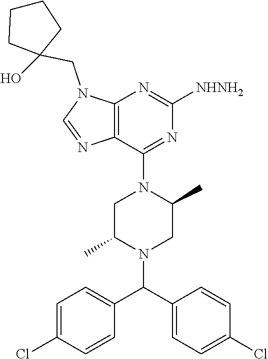 [SMILES: C[C@@H]1CN(c2nc(NN)nc3c2ncn3CC2(O)CCCC2)[C@@H](C)CN1C(c1ccc(Cl)cc1)c1ccc(Cl)cc1]

To a mixture of 1-((6-((2S,5R)-4-(bis(4-chlorophenyl)methyl)-2,5-dimethylpiperazin-1-yl)-2-chloro-9H-purin-9-yl)methyl)cyclopentan-1-ol (Step 3) and hydrazine hydrate (876 mg, 17.5 mmol) in 1,4-dioxane (5 mL) was added hydrazine (0.561 g, 17.5 mmol) and the mixture was purged with nitrogen and stirred at 120° C. overnight. After cooling to rt, the mixture was diluted with saturated aqueous NaHCO₃ and extracted with EtOAc. The organic layer was removed, and the aqueous layer was extracted with EtOAc. The combined organic layers were dried over MgSO₄, filtered, and concentrated. The crude residue was purified by flash column chromatography (SiO₂, 0-5% MeOH/CH₂Cl₂) to afford the desired product (0.26 g, 57% yield over 2 steps). LC-MS calculated for C₃₀H₃₇Cl₂N₈O (M+H)⁺: m/z=595.2. found 595.3.

### Step 5. 1-((4-((2S,5R)-4-(Bis(4-chlorophenyl)methyl)-2,5-dimethylpiperazin-1-yl)-1H-[1,2,4]triazolo[3,4-b]purin-1-yl)methyl)cyclopentan-1-ol

To a mixture of 1-((6-((2S,5R)-4-(bis(4-chlorophenyl)methyl)-2,5-dimethylpiperazin-1-yl)-2-hydrazineyl-9H-purin-9-yl)methyl)cyclopentan-1-ol (Step 4) in AcOH (4 mL) was added triethyl orthoformate (520 mg, 3.5 mmol) and the reaction mixture was stirred at 90° C. for 1 h. After cooling to rt, the mixture was diluted with saturated aqueous NaHCO₃ and extracted with EtOAc. The organic layer was removed, and the aqueous layer was extracted with EtOAc. The combined organic layers were dried over MgSO₄, filtered, and concentrated. The crude residue was purified by flash column chromatography (SiO₂, 0-5% MeOH/CH₂Cl₂). LC-MS calculated for C₃₁H₃₅Cl₂N₈O (M+H)⁺: m/z=605.2. found 605.3.

### Example 45. 1-((4-((2S,5R)-4-((4-Chlorophenyl)(3,3-difluorocyclobutyl)methyl)-2,5-dimethylpiperazin-1-yl)-1H-[1,2,4]triazolo[3,4-b]purin-1-yl)methyl)cyclopentan-1-ol

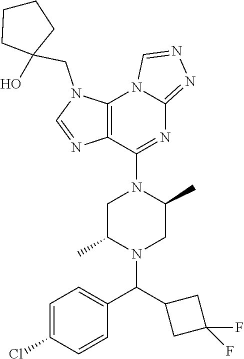

The title compound was prepared according to the procedures described in Example 44, with (2R,5S)-1-((4-chlorophenyl)(3,3-difluorocyclobutyl)methyl)-2,5-dimethylpiperazine hydrochloride (Intermediate 39) replacing (2R,5S)-1-(bis(4-chlorophenyl)methyl)-2,5-dimethylpiperazine hydrochloride in Step 1. In Step 5 after cooling to rt, the reaction mixture was diluted with acetonitrile, water, and several drops of TFA and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₉H₃₆ClF₂N₈O (M+H)⁺. m/z=585.3. found 585.3.

### Example 46. 1-((4-((2S,5R)-4-((3,3-Difluorocyclobutyl)(4-(trifluoromethyl)phenyl)methyl)-2,5-dimethylpiperazin-1-yl)-1H-[1,2,4]triazolo[3,4-b]purin-1-yl)methyl)cyclopentan-1-ol

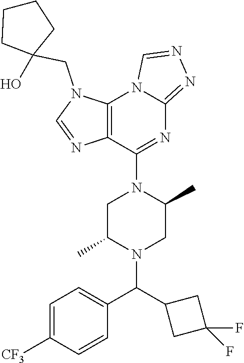 [SMILES: C[C@@H]1CN(c2nc3nncn3c3c2ncn3CC2(O)CCCC2)[C@@H](C)CN1C(c1ccc(C(F)(F)F)cc1)C1CC(F)(F)C1]

The title compound was prepared according to the procedures described in Example 44, with (2R,5S)-1-((3,3-difluorocyclobutyl)(4-(trifluoromethyl)phenyl)methyl)-2,5-dimethylpiperazine hydrochloride (Intermediate 3) replacing (2R,5S)-1-(bis(4-chlorophenyl)methyl)-2,5-dimethylpiperazine hydrochloride in Step 1. In Step 5 after cooling to rt, the reaction mixture was diluted with acetonitrile, water, and several drops of TFA and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₃₀H₃₆F₅N₈O (M+H)⁺: m/z=619.3. found 619.4.

### Example 47. 4-((2S,5R)-4-(4-Chlorobenzyl)-2,5-dimethylpiperazin-1-yl)-1-(((S)-tetrahydrofuran-2-yl)methyl)-1H-[1,2,4]triazolo[3,4-b]purine

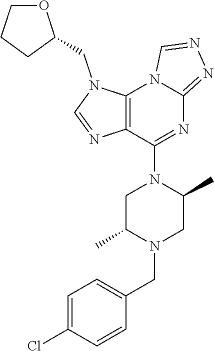 [SMILES: C[C@@H]1CN(c2nc3nncn3c3c2ncn3C[C@@H]2CCCO2)[C@@H](C)CN1Cc1ccc(Cl)cc1]

To a mixture of 4-((2S,5R)-2,5-dimethylpiperazin-1-yl)-1-(((S)-tetrahydrofuran-2-yl)methyl)-1H-[1,2,4]triazolo[3,4-b]purine hydrochloride (Intermediate 46, 10.0 mg, 0.023 mmol) in THF (1.0 mL) was added N,N-diisopropylethylamine (16.3 uL, 0.093 mmol) and 2-chlorobenzaldehyde (4.9 mg, 0.035 mmol) and the reaction mixture was stirred at rt for 10 min. AcOH (13.3 μL, 0.233 mmol) was added and the reaction mixture was stirred at rt for 10 min before sodium triacetoxyborohydride (14.8 mg, 0.070 mmol) was added and the reaction mixture was stirred at rt for 4 h. The reaction mixture was diluted with methanol and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₄H₃₀ClN₈O (M+H)⁺: m/z=481.2. found 481.2.

### Example 48. 4-((2S,5R)-4-(4-Chloro-2-fluorobenzyl)-2,5-dimethylpiperazin-1-yl)-1-(((S)-tetrahydrofuran-2-yl)methyl)-1H-[1,2,4]triazolo[3,4-b]purine

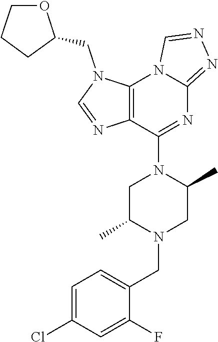 [SMILES: C[C@@H]1CN(c2nc3nncn3c3c2ncn3C[C@@H]2CCCO2)[C@@H](C)CN1Cc1ccc(Cl)cc1F]

The title compound was prepared according to the procedures described in Example 47, with 4-chloro-2-fluorobenzaldehyde replacing 4-chlorobenzaldehyde. LC-MS calculated for C₂₄H₂₉ClFN₈O (M+H)⁺: m/z=499.2. found 499.2.

### Example 49. 4-((2S,5R)-4-((3,3-Difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

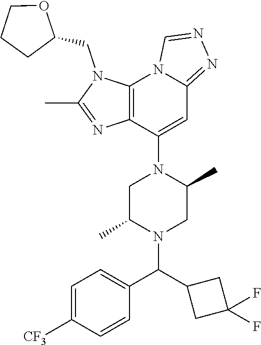 [SMILES: Cc1nc2c(N3C[C@@H](C)N(C(c4ccc(C(F)(F)F)cc4)C4CC(F)(F)C4)C[C@@H]3C)cc3nncn3c2n1C[C@@H]1CCCO1]

### Step 1. 6-Chloro-4-((2S,5R)-4-((3,3-difluorocyclobutyl)(4-(trifluoromethyl)phenyl)methyl)-2,5-dimethylpiperazin-1-yl)-3-nitropyridin-2-amine

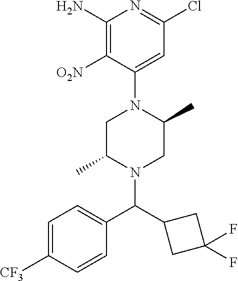 [SMILES: C[C@@H]1CN(c2cc(Cl)nc(N)c2[N+](=O)[O-])[C@@H](C)CN1C(c1ccc(C(F)(F)F)cc1)C1CC(F)(F)C1]

To a mixture of 4,6-dichloro-3-nitropyridin-2-amine (50 mg, 0.24 mmol, ChemScene CS-0094679) and (2R,5S)-1-((3,3-difluorocyclobutyl)(4-(trifluoromethyl)phenyl)methyl)-2,5-dimethylpiperazine hydrochloride (Intermediate 3, 96 mg, 0.24 mmol) in MeCN (2.0 mL) was added N,N-diisopropylethylamine (0.126 mL, 0.721 mmol) and the mixture was stirred at 90° C. overnight. After cooling to rt, the mixture was concentrated in vacuo, and the crude material obtained was used directly without further purification. LC-MS calculated for C₂₃H₂₆ClF₅N₅O₂ (M+H)⁺. m/z=534.2. found 534.3

### Step 2. 6-Chloro-4-((2S,5R)-4-((3,3-difluorocyclobutyl)(4-(trifluoromethyl)phenyl)methyl)-2,5-dimethylpiperazin-1-y)pyridine-2,3-diamine

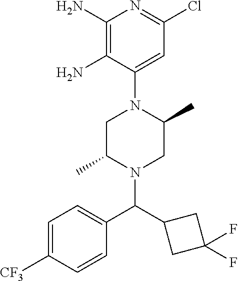 [SMILES: C[C@@H]1CN(c2cc(Cl)nc(N)c2N)[C@@H](C)CN1C(c1ccc(C(F)(F)F)cc1)C1CC(F)(F)C1]

A mixture of 6-chloro-4-((2S,5R)-4-((3,3-difluorocyclobutyl)(4-(trifluoromethyl)phenyl)methyl)-2,5-dimethylpiperazin-1-yl)-3-nitropyridin-2-amine (Step 1) in DMF (2.0 mL) was added hypodiboric acid (64.7 mg, 0.721 mmol), followed by dropwise addition of a solution of 4,4′-dipyridyl (0.38 mg, 2.4 μmol) in DMF (0.5 mL). The mixture was stirred at rt for 10 min, at which point the mixture was diluted with water and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₃H₂₈ClF₅N₅ (M+H)⁺: m/z=504.2. found 504.3.

### Step 3. 5-Chloro-7-((2S,5R)-4-((3,3-difluorocyclobutyl)(4-(trifluoromethyl)phenyl)methyl)-2,5-dimethylpiperazin-1-yl)-2-methyl-3H-imidazo[4,5-b]pyridine

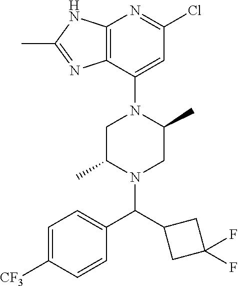 [SMILES: Cc1nc2c(N3C[C@@H](C)N(C(c4ccc(C(F)(F)F)cc4)C4CC(F)(F)C4)C[C@@H]3C)cc(Cl)nc2[nH]1]

A mixture of 6-chloro-4-((2S,5R)-4-((3,3-difluorocyclobutyl)(4-(trifluoromethyl)phenyl)methyl)-2,5-dimethylpiperazin-1-yl)pyridine-2,3-diamine (Step 2) and acetic acid (0.5 mL, 8.73 mmol) in triethyl orthoacetate (0.5 mL, 2.7 mmol) was stirred at 90° C. for 4 h. After cooling to rt, the mixture was diluted with saturated aqueous NaHCO₃ and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₅H₂₈ClF₅N₅ (M+H)⁺: m/z=528.2. found 528.2.

### Step 4. 5-Chloro-7-((2S,5R)-4-((3,3-difluorocyclobutyl)(4-(trifluoromethyl)phenyl)methyl)-2,5-dimethylpiperazin-1-yl)-2-methyl-3-(((S)-tetrahydrofuran-2-yl)methyl)-3H-imidazo[4,5-b]pyridine

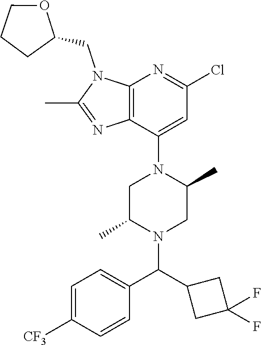 [SMILES: Cc1nc2c(N3C[C@@H](C)N(C(c4ccc(C(F)(F)F)cc4)C4CC(F)(F)C4)C[C@@H]3C)cc(Cl)nc2n1C[C@@H]1CCCO1]

A mixture of 5-chloro-7-((2S,5R)-4-((3,3-difluorocyclobutyl)(4-(trifluoromethyl)phenyl)methyl)-2,5-dimethylpiperazin-1-yl)-2-methyl-3H-imidazo[4,5-b]pyridine (Step 3) in MeCN (2.0 mL) was added cesium carbonate (236 mg, 0.724 mmol) and (S)-(tetrahydrofuran-2-yl)methyl methanesulfonate (Intermediate 50, 87 mg, 0.48 mmol) and the reaction mixture was stirred at 90° C. overnight. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₃₀H₃₆ClF₅N₅O (M+H)⁺. m/z=612.3. found 612.3.

### Step 5. 7-((2S,5R)-4-((3,3-Difluorocyclobutyl)(4-(trifluoromethyl)phenyl)methyl)-2,5-dimethylpiperazin-1-yl)-5-hydrazineyl-2-methyl-3-(((S)-tetrahydrofuran-2-yl)methyl)-3H-imidazo[4,5-b]pyridine

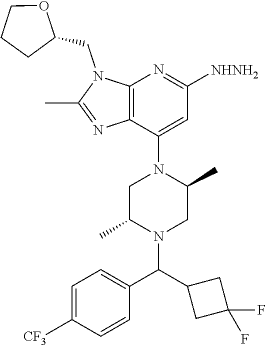 [SMILES: Cc1nc2c(N3C[C@@H](C)N(C(c4ccc(C(F)(F)F)cc4)C4CC(F)(F)C4)C[C@@H]3C)cc(NN)nc2n1C[C@@H]1CCCO1]

To a mixture of 5-chloro-7-((2S,5R)-4-((3,3-difluorocyclobutyl)(4-(trifluoromethyl)phenyl)methyl)-2,5-dimethylpiperazin-1-yl)-2-methyl-3-(((S)-tetrahydrofuran-2-yl)methyl)-3H-imidazo[4,5-b]pyridine (Step 4), cesium carbonate (0.236 g, 0.725 mmol), and methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (21 mg, 0.024 mmol, Aldrich 745979) in 1,4-dioxane (2 mL) was added hydrazine (0.077 mL, 2.4 mmol), and the mixture was purged with nitrogen and stirred at 90° C. for 1 h. After cooling to rt, the reaction mixture was diluted with CH₂Cl₂ and filtered through a pad of MgSO₄ in a SiliaPrep SPE thiol cartridge (500 mg, SiliCycle SPE-R51030B-06P). The filtrate was concentrated, and the crude residue was purified by flash column chromatography (12 g SiO₂, 0-5% MeOH/CH₂Cl₂) to afford the desired product (68 mg, 46% yield over 5 steps) as a mixture of diastereomers in the form of an off-white solid. LC-MS calculated for C₃₀H₃₉F₅N₇O (M+H)⁺. m/z=608.3. found 608.4.

### Step 6. 4-((2S,5R)-4-((3,3-Difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

To a mixture of 7-((2S,5R)-4-((3,3-difluorocyclobutyl)(4-(trifluoromethyl)phenyl)methyl)-2,5-dimethylpiperazin-1-yl)-5-hydrazineyl-2-methyl-3-(((S)-tetrahydrofuran-2-yl)methyl)-3H-imidazo[4,5-b]pyridine (68 mg, 0.11 mmol) in AcOH (1.0 mL, 17 mmol) was added triethyl orthoformate (0.037 mL, 0.224 mmol) and the reaction mixture was stirred at 90° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₃₁H₃₇F₅N₇O (M+H)⁺: m/z=618.3. found 618.4.

### Example 50. 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

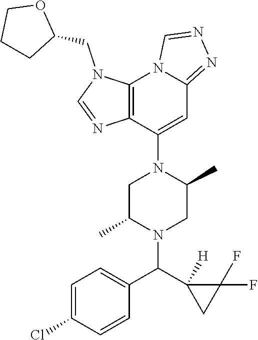

### Step 1. 7-((2S,5R)-4-((4-Chlorophenyl)((S)-2,2-difluorocyclopropyl)methyl)-2,5-dimethylpiperazin-1-yl)-5-hydrazineyl-3-(((S)-tetrahydrofuran-2-yl)methyl)-3H-imidazo[4,5-b]pyridine

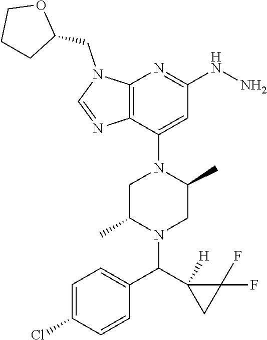

A mixture 5-chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine (Intermediate 52, 44.9 mg, 0.082 mmol), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (7.0 mg, 8.2 μmol, Aldrich 745979), cesium carbonate (80.0 mg, 0.245 mmol), and hydrazine hydrate (7.6 μL, 0.12 mmol, Aldrich 225819) in 1,4-dioxane (0.41 mL) was stirred at 90° C. for 1 h. The mixture was cooled to room temperature, filtered through a pad of MgSO₄ in a SiliaPrep SPE thiol cartridge (SiliCycle SPE-R51030B-06P), concentrated in vacuo, and purified by flash column chromatography (12 g SiO₂, MeOH/DCM) to give the title compound. LC-MS calculated for C₂₇H₃₅ClF₂N₇O (M+H)⁺: m/z=546.3. found 546.4.

### Step 2. 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

A mixture of 7-((2S,5R)-4-((4-chlorophenyl)((S)-2,2-difluorocyclopropyl)methyl)-2,5-dimethylpiperazin-1-yl)-5-hydrazineyl-3-(((S)-tetrahydrofuran-2-yl)methyl)-3H-imidazo[4,5-b]pyridine (Step 1), triethyl orthoformate (34 μL, 0.20 mmol), and acetic acid (117 μL, 2.04 mmol) was stirred at 95° C. for 1 h, cooled to room temperature, and directly purified by prep-HPLC (Sunfire C18 column, eluting with a gradient of acetonitrile/water containing 0.1% TFA, at flow rate of 60 mL/min) to afford the title compound as a single stereoisomer as its TFA salt. LC-MS calculated for C₂₈H₃₃ClF₂N₇O (M+H)⁺: m/z=556.2. found 556.3. ¹H NMR (500 MHz, DMSO-d₆) δ 9.55 (s, 1H), 8.25 (s, 1H), 7.48 (d, J=8.2 Hz, 2H), 7.43 (d, J=8.1 Hz, 2H), 6.54 (s, 1H), 5.08-4.91 (m, 2H), 4.84 (dd, J=15.1, 3.0 Hz, 1H), 4.63 (dd, J=15.1, 7.6 Hz, 1H), 4.23 (qd, J=7.2, 2.8 Hz, 1H), 3.74 (dd, J=13.1, 3.6 Hz, 1H), 3.64 (q, J=7.1 Hz, 1H), 3.61-3.55 (m, 2H), 3.38 (d, J=9.8 Hz, 1H), 2.85 (dd, J=12.2, 4.3 Hz, 1H), 2.28 (dd, J=12.2, 3.0 Hz, 1H), 2.21-2.07 (m, 2H), 1.88-1.68 (m, 3H), 1.51 (td, J=9.4, 5.2 Hz, 1H), 1.26 (d, J=6.5 Hz, 3H), 1.11 (d, J=6.4 Hz, 3H), 0.99 (dt, J=11.3, 8.3 Hz, 1H).

### Example 51. 4-((2S,5R)-4-(1-(4-Chlorophenyl)-3-methylbutyl)-2,5-dimethylpiperazin-1-yl)-1-(((S)-tetrahydrofuran-2-yl)methyl)-1H-imidazo[4,5-e][1,2,4]triazolo[4,3-a]pyridine

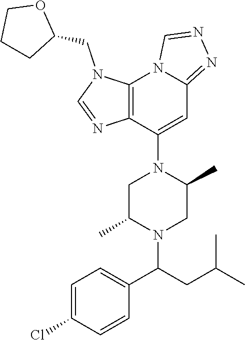

The title compound was prepared according to the procedures outlined in Example 50, with 5-chloro-7-((2S,5R)-4-(1-(4-chlorophenyl)-3-methylbutyl)-2,5-dimethylpiperazin-1-yl)-3-(((S)-tetrahydrofuran-2-yl)methyl)-3H-imidazo[4,5-b]pyridine (Intermediate 53) replacing 5-chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine in Step 1. The title compound was isolated as a single stereoisomer as its TFA salt. LC-MS calculated for C₂₉H₃₉ClN₇O (M+H)⁺: m/z=536.3. found 536.4.

### Example 52. 4-((2S,5R)-4-((4-Chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine

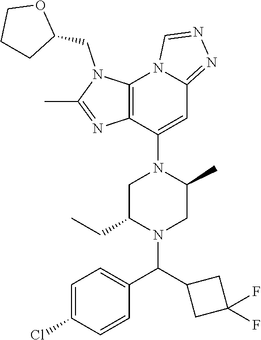

The title compound was prepared according to the procedures outlined in Example 49, with (2R,5S)-1-((4-chlorophenyl)(3,3-difluorocyclobutyl)methyl)-2-ethyl-5-methylpiperazine hydrochloride (Intermediate 43) replacing (2R,5S)-1-((3,3-difluorocyclobutyl)(4-(trifluoromethyl)phenyl)methyl)-2,5-dimethylpiperazine hydrochloride in Step 1. The title compound was isolated as a single stereoisomer as its TFA salt. LC-MS calculated for C₃₁H₃₉ClF₂N₇O (M+H)⁺. m/z=598.3. found 598.4. ¹H NMR (500 MHz, DMSO-d₆) (mixture of rotamers) δ 9.56 (s, 1H), 7.54-7.43 (m, 2H), 7.43-7.31 (m, 2H), δ 6.47 (s, 1H), 5.71-5.08 (m, 1H), 4.84-4.66 (m, 1.6H), 4.66-4.55 (m, 1.4H), 4.38-4.18 (m, 1H), 4.15-4.08 (m, 1H), 3.68-3.64 (m, 1H), 3.58-3.50 (m, 1H), 3.39-3.29 (m, 1H), 3.15-2.99 (m, 1H), 2.87-2.67 (m, 1H), 2.63-2.54 (m, 3H), 2.46-2.31 (m, 4H), 2.30-2.19 (m, 1H), 2.18-2.09 (m, 1H), 2.09-1.96 (m, 1H), 1.96-1.88 (m, 1H), 1.88-1.78 (m, 1H), 1.78-1.68 (m, 1H), 1.60-1.47 (m, 1H), 1.36-1.14 (m, 4H), 0.86-0.72 (m, 3H).

### Example 53. 4-((2S,5R)-4-(1-(4-Chlorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine

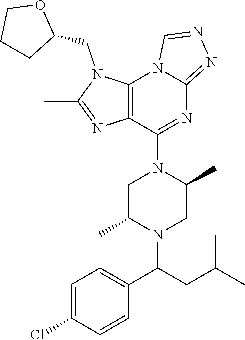

The title compound was prepared according to the procedures outlined in Steps 2-4 for Example 7, with (2R,5S)-4-(1-(4-chlorophenyl)-3-methylbutyl)-2,5-dimethylpiperazine hydrochloride (Intermediate 10) replacing (2R,5S)-1-((4-chlorophenyl)((R)-2,2-difluorocyclopropyl)methyl)-2,5-dimethylpiperazine hydrochloride and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The major diastereomer of the title compound was isolated as a single stereoisomer as its TFA salt. LC-MS calculated for C₂₉H₄₀ClN₈O (M+H)⁺. m/z=551.3. found 551.3.

### Example 54. 4-((2S,5R)-4-((4-Chlorophenyl)(cyclopropyl)methyl)-2,5-dimethylpiperazin-1-yl)-2-methyl-1-(((S)-tetrahydrofuran-2-yl)methyl)-1H-[1,2,4]triazolo[3,4-b]purine

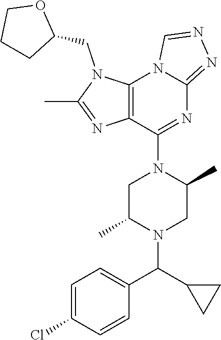

The title compound was prepared according to the procedures outlined for Example 7, with tert-butyl (2S,5R)-4-((4-chlorophenyl)(cyclopropyl)methyl)-2,5-dimethylpiperazine-1-carboxylate (Intermediate 14) replacing tert-butyl (2S,5R)-4-((4-chlorophenyl)((R)-2,2-difluorocyclopropyl)methyl)-2,5-dimethylpiperazine-1-carboxylate and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The title compound was isolated as a mixture of diastereomers as the TFA salts. LC-MS calculated for C₂₈H₃₆ClN₈O (M+H)⁺: m/z=535.3. found 535.3.

### Examples 55 and 56. 4-((2S,5R)-4-((S)-(4-Chlorophenyl)((R)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine and 4-((2S,5R)-4-((R)-(4-chlorophenyl)((R)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine

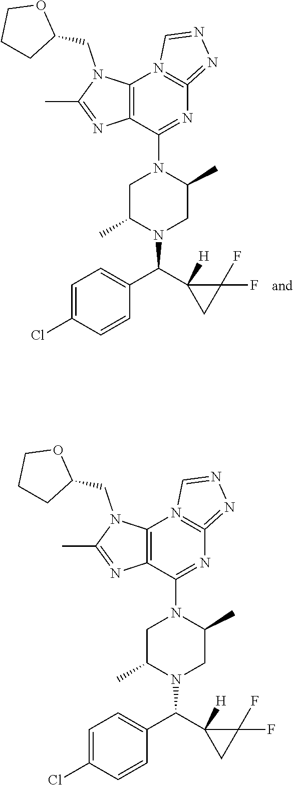

The title compounds were prepared according to the procedures outlined for Example 7, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. Following Step 2, the major and minor diastereomers were separated through flash column chromatography and separately subjected to Steps 3 and 4. Each title compound was isolated as a single stereoisomer as its TFA salt.

Example 55: Retention time on LC-MS tᵣ=1.407 min, LC-MS calculated for C₂₈H₃₄ClF₂N₈O (M+H)⁺: m/z=571.3. found 571.3.

Example 56: Retention time on LC-MS tᵣ=1.429 min, LC-MS calculated for C₂₈H₃₄ClF₂N₈O (M+H)⁺: m/z=571.3. found 571.3.

### Examples 57 and 58. 4-((2S,5R)-4-((S)-1-(4-Chlorophenyl)propyl)-2,5-dimethylpiperazin-1-yl)-1-(((S)-tetrahydrofuran-2-yl)methyl)-1H-[1,2,4]triazolo[3,4-b]purine and 4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine

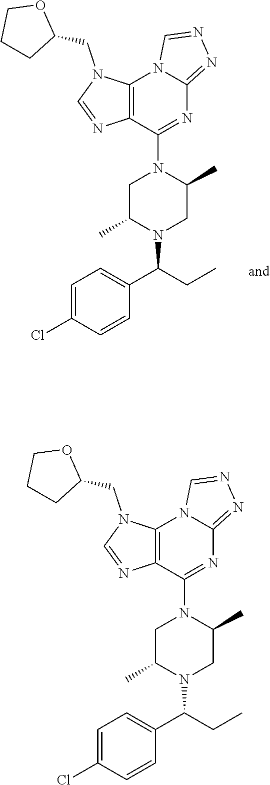 [SMILES: CC[C@@H](c1ccc(Cl)cc1)N1C[C@H](C)N(c2nc3nncn3c3c2ncn3C[C@@H]2CCCO2)C[C@H]1C ; CC[C@H](c1ccc(Cl)cc1)N1C[C@H](C)N(c2nc3nncn3c3c2ncn3C[C@@H]2CCCO2)C[C@H]1C]

The title compounds were prepared according to the procedures outlined in Steps 2-4 for Example 7, with (2R,5S)-1-(1-(4-chlorophenyl)propyl)-2,5-dimethylpiperazine hydrochloride (Intermediate 55) replacing (2R,5S)-1-((4-chlorophenyl)((R)-2,2-difluorocyclopropyl)methyl)-2,5-dimethylpiperazine hydrochloride. The crude reaction mixture was purified by prep-HPLC (Sunfire C18 column, eluting with a gradient of acetonitrile/water containing 0.1% TFA, at flow rate of 60 mL/min) to afford a diastereomeric mixture of the title compounds as TFA salts. The diastereomeric mixture was dissolved in CH₂Cl₂ (2 mL) and 1 M NaOH (5 mL) was added. The layers were separated and the aqueous layer was extracted with CH₂Cl₂ (5×2 mL). The combined organic layers were dried over MgSO₄ and concentrated in vacuo. The residue was purified by normal phase preparative chiral HPLC (CHIRALPAK® IG column, 250×21.2 mm, 5 μm, eluting with a gradient of 30% EtOH in hexanes, at a flow rate of 20 mL/min) to afford the title compounds as separated diastereomers. Each diastereomer was then taken up in MeCN (1 mL) and water (1 mL), several drops of TFA were added, and the mixture was frozen and dried via lyophilization to afford each of the title compounds as its TFA salt.

Example 57: Retention time on CHIRALPAK® IG column tᵣ=17 min, LC-MS calculated for C₂₆H₃₄ClN₈O (M+H)⁺: m/z=509.3. found 509.2.

Example 58: Retention time on CHIRALPAK® IG column tᵣ=24 min, LC-MS calculated for C₂₆H₃₄ClN₈O (M+H)⁺: m/z=509.3. found 509.2.

### Examples 59 and 60. 4-((2S,5R)-2,5-Dimethyl-4-((S)-1-(4-(trifluoromethyl)phenyl)propyl)piperazin-1-yl)-1-(((S)-tetrahydrofuran-2-yl)methyl)-1H-[1,2,4]triazolo[3,4-b]purine and 4-((2S,5R)-2,5-dimethyl-4-((R)-1-(4-(trifluoromethyl)phenyl)propyl)piperazin-1-yl)-1-(((S)-tetrahydrofuran-2-yl)methyl)-1H-[1,2,4]triazolo[3,4-b]purine

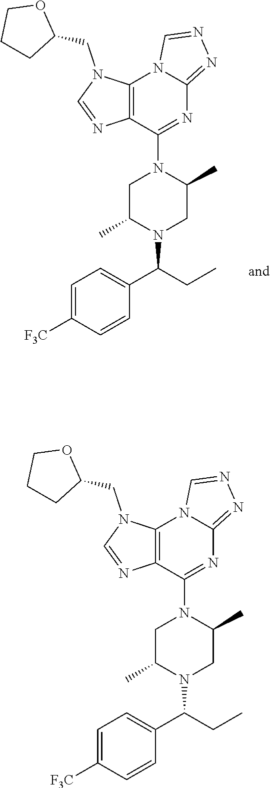 [SMILES: CC[C@@H](c1ccc(C(F)(F)F)cc1)N1C[C@H](C)N(c2nc3nncn3c3c2ncn3C[C@@H]2CCCO2)C[C@H]1C ; CC[C@H](c1ccc(C(F)(F)F)cc1)N1C[C@H](C)N(c2nc3nncn3c3c2ncn3C[C@@H]2CCCO2)C[C@H]1C]

The title compounds were prepared according to the procedures outlined in Steps 2-4 for Example 7, with (2R,5S)-1-(1-(4-(trifluoromethyl)phenyl)propyl)-2,5-dimethylpiperazine hydrochloride (Intermediate 57) replacing (2R,5S)-1-((4-chlorophenyl)((R)-2,2-difluorocyclopropyl)methyl)-2,5-dimethylpiperazine hydrochloride. The crude reaction mixture was purified by prep-HPLC (Sunfire C18 column, eluting with a gradient of acetonitrile/water containing 0.1% TFA, at flow rate of 60 mL/min) to afford a diastereomeric mixture of the title compounds as TFA salts. The diastereomeric mixture was further purified by normal phase preparative chiral HPLC (Phenomenex LUX Amylose-2 column, 250×21.2 mm, 5 μm, eluting with a gradient of 30% EtOH in hexanes, at a flow rate of 20 mL/min) to afford the title compounds as separated diastereomers.

Example 59: Retention time on Phenomenex LUX Amylose-2 column column tᵣ=24 min., LC-MS calculated for C₂₇H₃₄F₃N₈O (M+H)⁺: m/z=543.3. found 543.2.

Example 60: Retention time on Phenomenex LUX Amylose-2 column column tᵣ=26 min., LC-MS calculated for C₂₇H₃₄F₃N₈O (M+H)⁺: m/z=543.3. found 543.2.

### Example 61. 4-((2S,5R)-4-((4-Chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

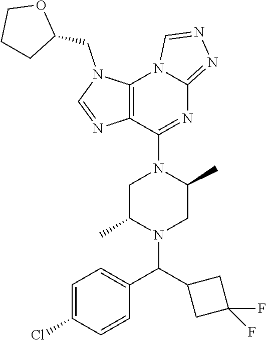

The title compound was prepared according to the procedures outlined in Example 50, with 5-chloro-7-((2S,5R)-4-((4-chlorophenyl)(3,3-difluorocyclobutyl)methyl)-2,5-dimethylpiperazin-1-yl)-3-(((S)-tetrahydrofuran-2-yl)methyl)-3H-imidazo[4,5-b]pyridine (Intermediate 58) replacing 5-chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine in Step 1. The major diastereomer of the title compound was isolated as a single stereoisomer as its TFA salt. LC-MS calculated for C₂₉H₃₅ClF₂N₇O (M+H)⁺: m/z=570.3. found 570.3. ¹H NMR (500 MHz, DMSO-d₆) δ 9.63 (s, 1H), 8.31 (s, 1H), 7.50-7.44 (m, 4H), 6.58 (s, 1H), 5.32-4.72 (m, 3H), 4.63 (dd, J=15.1, 7.7 Hz, 1H), 4.20 (qd, J=7.0, 2.8 Hz, 1H), 3.75-3.37 (m, 4H), 3.25-3.00 (m, 1H), 2.94-2.76 (m, 2H), 2.75-2.55 (m, 2H), 2.48-2.30 (m, 1H), 2.28-2.15 (m, 1H), 2.13-1.96 (m, 2H), 1.85-1.68 (m, 3H), 1.34 (d, J=6.4 Hz, 3H), 1.15-0.87 (m, 3H).

### Example 62. 4-((2S,5R)-4-((4-Chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

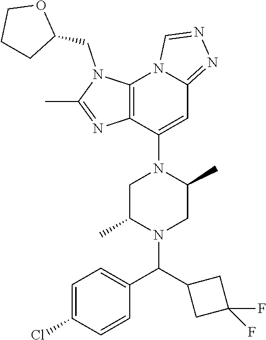

The title compound was prepared according to the procedures outlined in Example 50, with 5-chloro-7-((2S,5R)-4-((4-chlorophenyl)(3,3-difluorocyclobutyl)methyl)-2,5-dimethylpiperazin-1-yl)-2-methyl-3-(((S)-tetrahydrofuran-2-yl)methyl)-3H-imidazo[4,5-b]pyridine (Intermediate 59) replacing 5-chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine in Step 1. The major diastereomer of the title compound was isolated as a single stereoisomer as its TFA salt. LC-MS calculated for C₃₀H₃₇ClF₂N₇O (M+H)⁺: m/z=584.3. found 584.2. ¹H NMR (500 MHz, DMSO-d₆) δ 9.59 (s, 1H), 7.56-7.35 (m, 4H), 6.56 (s, 1H), 5.33-4.68 (m, 3H), 4.63 (dd, J=15.9, 9.1 Hz, 1H), 4.12 (q, J=9.0 Hz, 1H), 3.79-3.51 (m, 3H), 3.51-3.36 (m, 1H), 3.29-3.00 (m, 1H), 2.99-2.76 (m, 2H), 2.72-2.57 (m, 2H), 2.51 (s, 3H), 2.48-2.32 (m, 1H), 2.27-2.10 (m, 2H), 2.08-1.97 (m, 1H), 1.97-1.89 (m, 1H), 1.83 (dp, J=11.6, 7.5 Hz, 1H), 1.74 (dq, J=12.0, 8.2 Hz, 1H), 1.33 (d, J=6.2 Hz, 3H), 1.11-0.90 (m, 3H).

### Examples 63 and 64. 4-((2S,5R)-2,5-Dimethyl-4-((S)-2-methyl-1-(6-(trifluoromethyl)quinolin-2-yl)propyl)piperazin-1-yl)-2-methyl-1-(((S)-tetrahydrofuran-2-yl)methyl)-1H-[1,2,4]triazolo[3,4-b]purine and 4-((2S,5R)-2,5-dimethyl-4-((R)-2-methyl-1-(6-(trifluoromethyl)quinolin-2-yl)propyl)piperazin-1-yl)-2-methyl-1-(((S)-tetrahydrofuran-2-yl)methyl)-1H-[1,2,4]triazolo[3,4-b]purine

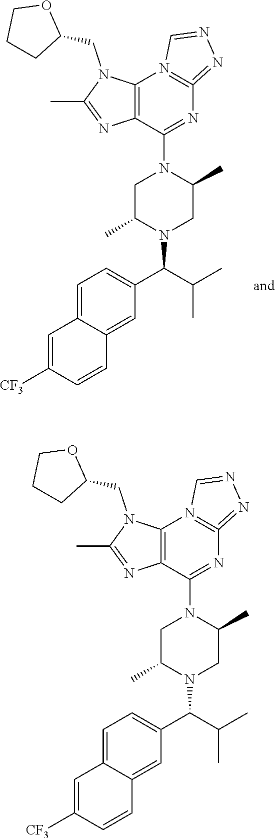

The title compounds were prepared according to the procedures described in Example 23, with 2-(1-((2R,5S)-2,5-dimethylpiperazin-1-yl)-2-methylpropyl)-6-(trifluoromethyl)quinoline dihydrochloride (Intermediate 61) replacing (2R,5S)-1-(1-(4-chloro-3-fluorophenyl)-2-methylpropyl)-2,5-dimethylpiperazine dihydrochloride in Step 1.

Example 63: Retention time on LC-MS tᵣ=1.21 min. LC-MS calculated for C₃₂H₃₉F₃N₉O (M+H)⁺: m/z=622.3. found 622.4.

Example 64: Retention time on LC-MS tᵣ=1.33 min. LC-MS calculated for C₃₂H₃₉F₃N₉O (M+H)⁺: m/z=622.3. found 622.4.

### Example 65 and 66. 4-((2S,5R)-4-((S)-1-(4-Chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine and 4-((2S,5R)-4-((R)-1-(4-chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

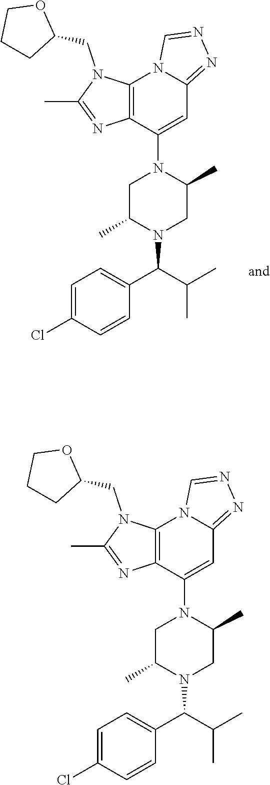 [SMILES: Cc1nc2c(N3C[C@@H](C)N([C@H](c4ccc(Cl)cc4)C(C)C)C[C@@H]3C)cc3nncn3c2n1C[C@@H]1CCCO1 ; Cc1nc2c(N3C[C@@H](C)N([C@@H](c4ccc(Cl)cc4)C(C)C)C[C@@H]3C)cc3nncn3c2n1C[C@@H]1CCCO1]

### Step 1. 6-Chloro-4-((2S,5R)-4-(1-(4-chlorophenyl)-2-methylpropyl)-2,5-dimethylpiperazin-1-yl)-3-nitro-N-(((S)-tetrahydrofuran-2-yl)methyl)pyridin-2-amine

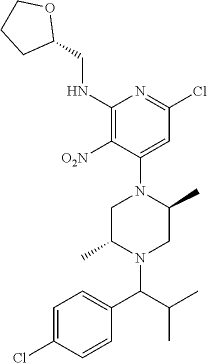 [SMILES: CC(C)C(c1ccc(Cl)cc1)N1C[C@H](C)N(c2cc(Cl)nc(NC[C@@H]3CCCO3)c2[N+](=O)[O-])C[C@H]1C]

To a mixture of 4,6-dichloro-3-nitropyridin-2-amine (20.0 mg, 0.88 mmol, ChemScene CS-0094679) and (2R,5S)-1-(1-(4-chlorophenyl)-2-methylpropyl)-2,5-dimethylpiperazine dihydrochloride (311 mg, 0.88 mmol) in MeCN (10.0 mL) was added N,N-diisopropylethylamine (0.614 mL, 3.525 mmol) and the mixture was stirred at rt overnight. To the reaction mixture was added (S)-(tetrahydrofuran-2-yl)methyl methanesulfonate (Intermediate 50, 0.191 g, 1.06 mmol). The resulting mixture was stirred at 100° C. overnight. After cooling to rt, the mixture was washed with saturated aqueous NaHCO₃, extracted with ethyl acetate, dried over MgSO₄, filtered and concentrated. The crude material obtained was used directly without further purification. LC-MS calculated for C₂₆H₃₆Cl₂N₅O₃ (M+H)⁺. m/z=536.2. found 536.2.

### Step 2. 6-Chloro-4-((2S,5R)-4-(1-(4-chlorophenyl)-2-methylpropyl)-2,5-dimethylpiperazin-1-yl)-N²-(((S-tetrahydrofuran-2-yl)methyl)pyridine-2,3-diamine

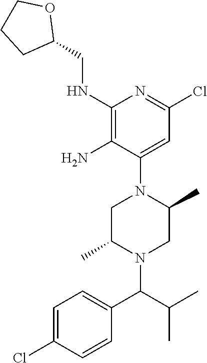 [SMILES: CC(C)C(c1ccc(Cl)cc1)N1C[C@H](C)N(c2cc(Cl)nc(NC[C@@H]3CCCO3)c2N)C[C@H]1C]

To a mixture of 6-chloro-4-((2S,5R)-4-(1-(4-chlorophenyl)-2-methylpropyl)-2,5-dimethylpiperazin-1-yl)-3-nitro-N—(((S)-tetrahydrofuran-2-yl)methyl)pyridin-2-amine (Step 1) in MeOH (4.0 mL) and THF (10.0 mL) was added hypodiboric acid (0.237 g, 2.64 mmol), followed by 4,4′-dipyridyl (27.0 mg, 0.176 mmol). The mixture was stirred at rt for 10 min, at which point the mixture was diluted with water and EtOAc. The organic layer was removed, and the aqueous layer was extracted with EtOAc. The combined organic layers were dried over MgSO₄, filtered, and concentrated under reduced pressure to afford the desired product. The crude residue was purified by flash column chromatography (40 g SiO₂, 0-5% MeOH/CH₂Cl₂) to afford the desired product (243 mg, 55% yield over 3 steps) as a mixture of diastereomers in the form of an off-white solid. LC-MS calculated for C₂₆H₃₈Cl₂N₅O (M+H)⁺: m/z=506.2. found 506.3.

### Step 3. 5-Chloro-7-((2S,5R)-4-(1-(4-chlorophenyl)-2-methylpropyl)-2,5-dimethylpiperazin-1-yl)-2-methyl-3-(((S)-tetrahydrofuran-2-yl)methyl)-3H-imidazo[4,5-b]pyridine

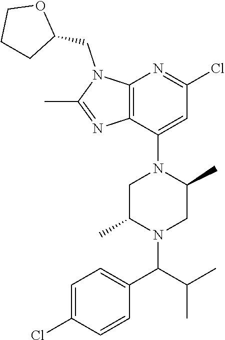 [SMILES: Cc1nc2c(N3C[C@@H](C)N(C(c4ccc(Cl)cc4)C(C)C)C[C@@H]3C)cc(Cl)nc2n1C[C@@H]1CCCO1]

A mixture of 6-chloro-4-((2S,5R)-4-(1-(4-chlorophenyl)-2-methylpropyl)-2,5-dimethylpiperazin-1-yl)-N²-(((S)-tetrahydrofuran-2-yl)methyl)pyridine-2,3-diamine (Step 2) and triethyl orthoacetate (0.29 g, 1.76 mmol) in acetic acid (1.0 mL, 17.6 mmol) was stirred at 95° C. for 2 h. After cooling to rt, the mixture was diluted with saturated aqueous NaHCO₃ and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₈H₃₈Cl₂N₅O (M+H)⁺. m/z=530.2. found 530.2.

### Step 4. 7-((2S,5R)-4-(1-(4-Chlorophenyl)-2-methylpropyl)-2,5-dimethylpiperazin-1-yl)-5-hydrazineyl-2-methyl-3-(((S)-tetrahydrofuran-2-yl)methyl)-3H-imidazo[4,5-b]pyridine

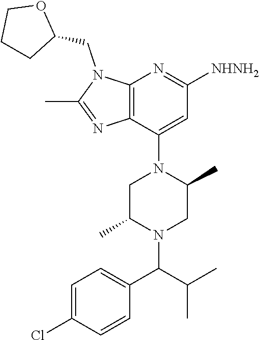 [SMILES: Cc1nc2c(N3C[C@@H](C)N(C(c4ccc(Cl)cc4)C(C)C)C[C@@H]3C)cc(NN)nc2n1C[C@@H]1CCCO1]

To a mixture of 5-chloro-7-((2S,5R)-4-(1-(4-chlorophenyl)-2-methylpropyl)-2,5-dimethylpiperazin-1-yl)-2-methyl-3-(((S)-tetrahydrofuran-2-yl)methyl)-3H-imidazo[4,5-b]pyridine (Step 3), cesium carbonate (0.57 g, 1.76 mmol), and methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (75.0 mg, 0.088 mmol, Aldrich 745979) in 1,4-dioxane (2.0 mL) was added hydrazine (0.28 mL, 8.79 mmol), and the mixture was purged with nitrogen and stirred at 60° C. for 1 h. After cooling to rt, the reaction mixture was diluted with CH₂Cl₂ and filtered through a pad of MgSO₄ in a SiliaPrep SPE thiol cartridge (500 mg, SiliCycle SPE-R51030B-06P). The filtrate was concentrated, and the crude residue was purified by flash column chromatography (40 g SiO₂, 0-5% MeOH/CH₂Cl₂). LC-MS calculated for C₂₈H₄₁ClN₇O (M+H)⁺: m/z=526.3. found 526.4.

### Step 5. 4-((2S,5R)-4-((S)-1-(4-Chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine and 4-((2S,5R)-4-((R)-1-(4-chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

To a mixture of 7-((2S,5R)-4-(1-(4-chlorophenyl)-2-methylpropyl)-2,5-dimethylpiperazin-1-yl)-5-hydrazineyl-2-methyl-3-(((S)-tetrahydrofuran-2-yl)methyl)-3H-imidazo[4,5-b]pyridine (Step 4) in acetic acid (1.01 mL, 17.6 mmol) was added triethyl orthoformate (0.652 g, 4.40 mmol). The reaction mixture was stirred at 95° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt.

Example 65: Retention time on LC-MS tᵣ=0.48 min. LC-MS calculated for C₂₉H₃₉ClN₇O (M+H)⁺: m/z=536.3. found 536.3.

Example 66: Retention time on LC-MS tᵣ=0.50 min. LC-MS calculated for C₂₉H₃₉ClN₇O (M+H)⁺: m/z=536.3. found 536.3.

### Example 67. 4-((2S,5R)-2,5-Dimethyl-4-(1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine

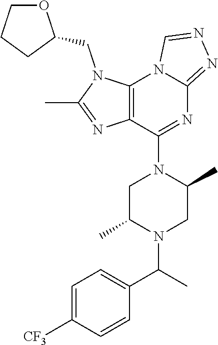 [SMILES: Cc1nc2c(N3C[C@@H](C)N(C(C)c4ccc(C(F)(F)F)cc4)C[C@@H]3C)nc3nncn3c2n1C[C@@H]1CCCO1]

The title compound was prepared according to the procedures described in Examples 41 and 42, with tert-butyl (2S,5R)-2,5-dimethylpiperazine-1-carboxylate replacing tert-butyl (2S,5R)-5-ethyl-2-methylpiperazine-1-carboxylate in Step 2. The title compound was purified by prep-HPLC (Sunfire C18 column, eluting with a gradient of acetonitrile/water containing 0.1% TFA, at flow rate of 60 mL/min) to afford a mixture of diastereomers as TFA salts. LC-MS calculated for C₂₇H₃₄F₃N₈O (M+H)⁺: m/z=543.3. found 543.3.

### Example 68. 4-((2S,5R)-2,5-Dimethyl-4-(1-(4-(trifluoromethyl)phenyl)ethyl)piperazin-1-yl)-1-(((S)-tetrahydrofuran-2-yl)methyl)-1H-[1,2,4]triazolo[3,4-b]purine

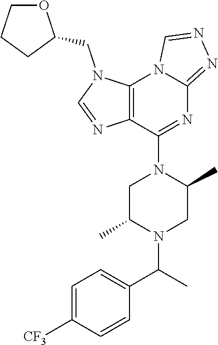 [SMILES: CC(c1ccc(C(F)(F)F)cc1)N1C[C@H](C)N(c2nc3nncn3c3c2ncn3C[C@@H]2CCCO2)C[C@H]1C]

The title compound was prepared according to the procedures described in Examples 41 and 42, with tert-butyl (2S,5R)-2,5-dimethylpiperazine-1-carboxylate replacing tert-butyl (2S,5R)-5-ethyl-2-methylpiperazine-1-carboxylate in Step 2, and (S)-2,6-dichloro-9-((tetrahydrofuran-2-yl)methyl)-9H-purine (Intermediate 1) replacing (S)-2,6-dichloro-8-methyl-9-((tetrahydrofuran-2-yl)methyl)-9H-purine (Intermediate 5) in Step 4. The title compound was purified by prep-HPLC (Sunfire C18 column, eluting with a gradient of acetonitrile/water containing 0.1% TFA, at flow rate of 60 mL/min) to afford a mixture of diastereomers as TFA salts. LC-MS calculated for C₂₆H₃₂F₃N₈O (M+H)⁺: m/z=529.3. found 529.3.

### Example 69. 4-((2S,5R)-4-(2-Fluoro-4-(trifluoromethyl)benzyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

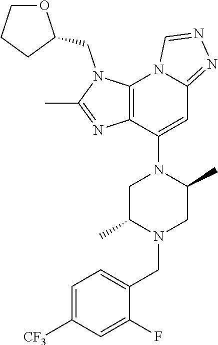 [SMILES: Cc1nc2c(N3C[C@@H](C)N(Cc4ccc(C(F)(F)F)cc4F)C[C@@H]3C)cc3nncn3c2n1C[C@@H]1CCCO1]

The title compound was prepared according to the procedures described in Example 37, with 4-((2S,5R)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine hydrochloride (Intermediate 47) replacing 4-((2S,5R)-2,5-dimethylpiperazin-1-yl)-2-methyl-1-(((S)-tetrahydrofuran-2-yl)methyl)-1H-[1,2,4]triazolo[3,4-b]purine hydrochloride. LC-MS calculated for C₂₇H₃₂F₄N₇O (M+H)⁺: m/z=546.3. found 546.3.

### Examples 70-76

The compounds in Table 2 were prepared according to the procedures described in Example 37 using the indicated aldehyde starting material.

TABLE 2

| | | | Aldehyde Starting | Analytical |
|---|---|---|---|---|
| Ex. | Name | Structure | Material | data |
| 70 | 4-((2S,5R)-4-(4- Chlorobenzyl)-2,5- dimethylpiperazin- 1-yl)-2-methyl-1- (((S)- tetrahydrofuran-2- yl)methyl)-1H- [1,2,4]triazolo[3,4- b]purine | 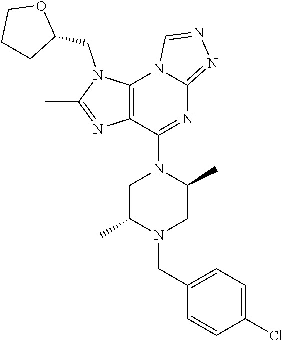 [SMILES: Cc1nc2c(N3C[C@@H](C)N(Cc4ccc(Cl)cc4)C[C@@H]3C)nc3nncn3c2n1C[C@@H]1CCCO1] | 4-Chlorobenzaldehyde | LC-MS calculated for C₂₅H₃₂ClN₈O (M + H)⁺: m/z = 495.2; found 495.3. |
| 71 | 4-((2S,5R)-4-(4- Chloro-2- fluorobenzyl)-2,5- dimethylpiperazin- 1-yl)-2-methyl-1- (((S)- tetrahydrofuran-2- yl)methyl)-1H- [1,2,4]triazolo[3,4- b]purine | 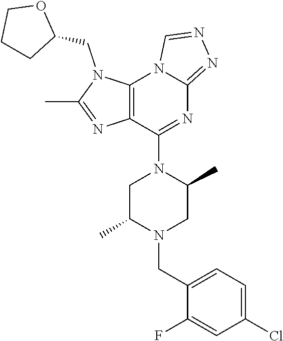 [SMILES: Cc1nc2c(N3C[C@@H](C)N(Cc4ccc(Cl)cc4F)C[C@@H]3C)nc3nncn3c2n1C[C@@H]1CCCO1] | 4-Chloro-2- fluorobenzaldehyde | LC-MS calculated for C₂₅H₃₁ClFN₈O (M + H)⁺: m/z = 513.2; found 513.2. |
| 72 | 4-((2S,5R)-4-(3,4- Dichlorobenzyl)- 2,5- dimethylpiperazin- 1-yl)-2-methyl-1- (((S)- tetrahydrofuran-2- yl)methyl)-1H- [1,2,4]triazolo[3,4- b]purine | 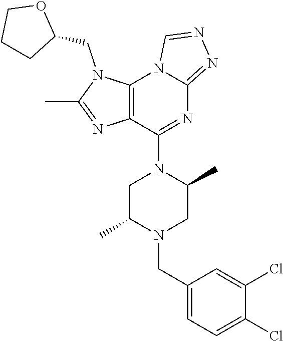 [SMILES: Cc1nc2c(N3C[C@@H](C)N(Cc4ccc(Cl)c(Cl)c4)C[C@@H]3C)nc3nncn3c2n1C[C@@H]1CCCO1] | 3,4- Dichlorobenzaldehyde | LC-MS calculated for C₂₅H₃₁Cl₂N₈O (M + H)⁺: m/z = 529.2; found 529.3. |
| 73 | 4-((2S,5R)-4-(4- Chloro-2,6- difluorobenzyl)-2,5- dimethylpiperazin- 1-yl)-2-methyl-1- (((S)- tetrahydrofuran-2- yl)methyl)-1H- [1,2,4]triazolo[3,4- b]purine | 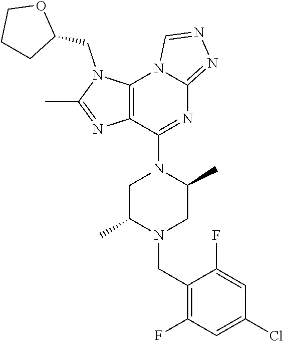 [SMILES: Cc1nc2c(N3C[C@@H](C)N(Cc4c(F)cc(Cl)cc4F)C[C@@H]3C)nc3nncn3c2n1C[C@@H]1CCCO1] | 4-Chloro-2,6- difluorobenzaldehyde | LC-MS calculated for C₂₅H₃₀ClF₂N₈O (M + H)⁺: m/z = 531.2; found 531.3. |
| 74 | 4-((2S,5R)-4-(4- Chloro-3-fluoro-5- (trifluoromethyl) benzyl)-2,5- dimethylpiperazin- 1-y1)-2-methyl-1- (((S)- tetrahydrofuran-2- yl)methyl)-1H- [1,2,4]triazolo[3,4- b]purine | 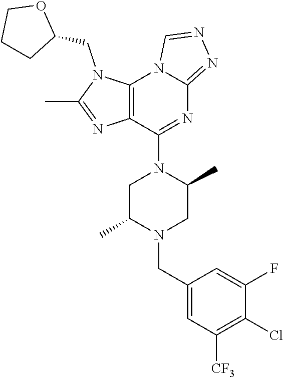 [SMILES: Cc1nc2c(N3C[C@@H](C)N(Cc4cc(F)c(Cl)c(C(F)(F)F)c4)C[C@@H]3C)nc3nncn3c2n1C[C@@H]1CCCO1] | 4-Chloro-3-fluoro-5- (trifluoromethyl) benzaldehyde | LC-MS calculated for C₂₆H₃₀ClF₄N₈O (M + H)⁺: m/z = 581.2; found 581.3. |
| 75 | 4-((2S,5R)-4-(3- Fluoro-4- (trifluoromethyl) benzyl)-2,5- dimethylpiperazin- 1-yl)-2-methyl-1- (((S)- tetrahydrofuran-2- yl)methyl)-1H- [1,2,4]triazolo[3,4- b]purine | 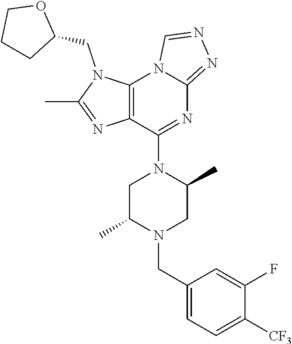 [SMILES: Cc1nc2c(N3C[C@@H](C)N(Cc4ccc(C(F)(F)F)c(F)c4)C[C@@H]3C)nc3nncn3c2n1C[C@@H]1CCCO1] | 3-Fluoro-4- (trifluoromethyl) benzaldehyde | LC-MS calculated for C₂₆H₃₁F₄N₈O (M + H)⁺: m/z = 547.3; found 547.4. |
| 76 | 4-((2S,5R)-4-(4- Chloro-3- fluorobenzyl)-2,5- dimethylpiperazin- 1-yl)-2-methyl-1- (((S)- tetrahydrofuran-2- yl)methyl)-1H- [1,2,4]triazolo[3,4- b]purine | 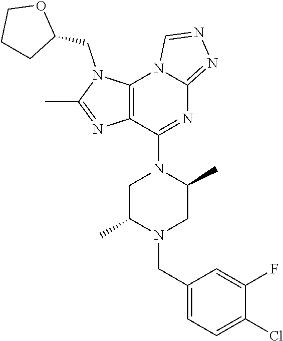 [SMILES: Cc1nc2c(N3C[C@@H](C)N(Cc4ccc(Cl)c(F)c4)C[C@@H]3C)nc3nncn3c2n1C[C@@H]1CCCO1] | 4-Chloro-3- fluorobenzaldehyde | LC-MS calculated for C₂₅H₃₁ClFN₈O (M + H)⁺: m/z = 513.2; found 513.2. |

### Example 77. 4-((2S,5R)-4-(4-Chloro-3-fluorobenzyl)-2,5-dimethylpiperazin-1-yl)-1-(((S)-tetrahydrofuran-2-yl)methyl)-1H-[1,2,4]triazolo[3,4-b]purine

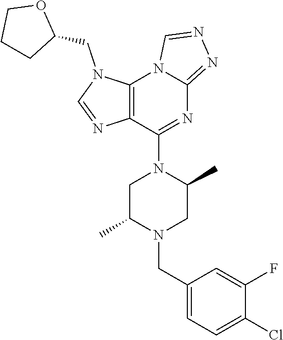 [SMILES: C[C@@H]1CN(c2nc3nncn3c3c2ncn3C[C@@H]2CCCO2)[C@@H](C)CN1Cc1ccc(Cl)c(F)c1]

The title compound was prepared according to the procedures described in Example 38, with 4-chloro-3-fluorobenzaldehyde replacing 2-fluoro-4-(trifluoromethyl)benzaldehyde. LC-MS calculated for C₂₄H₂₉ClFN₈O (M+H)⁺. m/z=499.2. found 499.2.

### Example 78. 4-((2S,5R)-4-((4-Chlorophenyl)(3,3-difluorocyclobutyl)methyl)-5-ethyl-2-methylpiperazin-1-yl)-2-methyl-1-(((S)-tetrahydrofuran-2-yl)methyl)-1H-[1,2,4]triazolo[3,4-b]purine

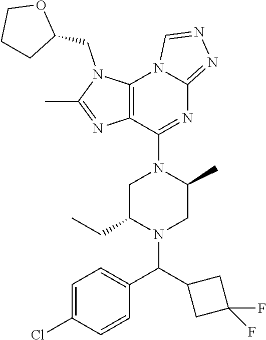 [SMILES: CC[C@@H]1CN(c2nc3nncn3c3c2nc(C)n3C[C@@H]2CCCO2)[C@@H](C)CN1C(c1ccc(Cl)cc1)C1CC(F)(F)C1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (2R,5S)-1-((4-Chlorophenyl)(3,3-difluorocyclobutyl)methyl)-2-ethyl-5-methylpiperazine hydrochloride (Intermediate 43) replacing (2R,5S)-1-((3,3-difluorocyclobutyl)(4-(trifluoromethyl)phenyl)methyl)-2,5-dimethylpiperazine hydrochloride in Step 1. LC-MS calculated for C₃₀H₃₈ClF₂N₈O (M+H)⁺: m/z=599.3. found 599.3.

### Example 79. 4-((2S,5R)-4-((4-Chloro-2,5-difluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine

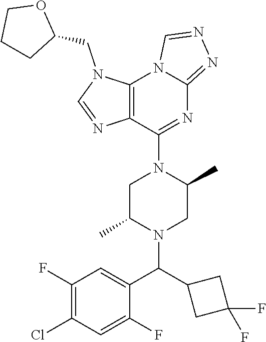 [SMILES: C[C@@H]1CN(c2nc3nncn3c3c2ncn3C[C@@H]2CCCO2)[C@@H](C)CN1C(c1cc(F)c(Cl)cc1F)C1CC(F)(F)C1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (2R,5S)-1-((4-chloro-2,5-difluorophenyl)(3,3-difluorocyclobutyl)methyl)-2,5-dimethylpiperazine hydrochloride (Intermediate 62) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₉H₃₄ClF₄N₈O (M+H)⁺: m/z=621.3. found 621.3.

### Example 80. 4-((2S,5R)-4-((4-Chloro-2,3-difluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine

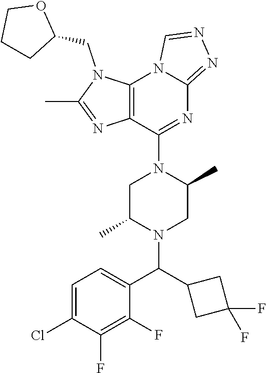 [SMILES: Cc1nc2c(N3C[C@@H](C)N(C(c4ccc(Cl)c(F)c4F)C4CC(F)(F)C4)C[C@@H]3C)nc3nncn3c2n1C[C@@H]1CCCO1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (2R,5S)-1-((4-chloro-2,3-difluorophenyl)(3,3-difluorocyclobutyl)methyl)-2,5-dimethylpiperazine hydrochloride (Intermediate 63) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₉H₃₄ClF₄N₈O (M+H)⁺. m/z=621.3. found 621.3.

### Example 81. 4-((1R,5S)-8-((3,3-Difluorocyclobutyl)(4-(trifluoromethyl)phenyl)methyl)-3,8-diazabicyclo[3.2.1]octan-3-yl)-2-methyl-1-(((S)-tetrahydrofuran-2-yl)methyl)-1H-[1,2,4]triazolo[3,4-b]purine

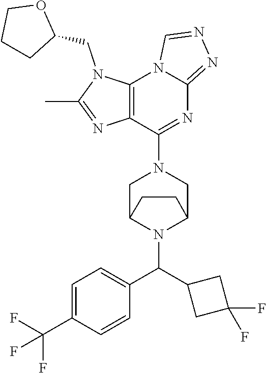 [SMILES: Cc1nc2c(N3CC4CCC(C3)N4C(c3ccc(C(F)(F)F)cc3)C3CC(F)(F)C3)nc3nncn3c2n1C[C@@H]1CCCO1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (1R,5S)-8-((3,3-difluorocyclobutyl)(4-(trifluoromethyl)phenyl)methyl)-3,8-diazabicyclo[3.2.1]octane hydrochloride (Intermediate 65) replacing (2R,5S)-1-((3,3-difluorocyclobutyl)(4-(trifluoromethyl)phenyl)methyl)-2,5-dimethylpiperazine hydrochloride in Step 1. LC-MS calculated for C₃₀H₃₄F₅N₈O (M+H)⁺: m/z=617.3. found 617.4. ¹H NMR (600 MHz, DMSO-d₆) (mixture of rotamers) δ 9.57 (s, 1H), 7.94-7.74 (m, 4H), 6.11-5.90 (m, 0.5H), 5.89-5.75 (m, 0.5H), 4.83-4.70 (m, 1.5H), 4.69-4.55 (m, 1.5H), 4.54-4.46 (m, 1H), 4.17-4.09 (m, 1.5H), 4.06-3.93 (m, 0.5H), 3.91-3.78 (m, 0.5H), 3.75-3.64 (m, 1.5H), 3.64-3.40 (m, 2.5H), 3.18-3.04 (m, 1.5H), 3.05-2.87 (m, 1H), 2.83-2.68 (m, 1H), 2.68-2.54 (m, 3H), 2.40-2.23 (m, 1H), 2.23-2.01 (m, 4H), 2.00-1.90 (m, 1H), 1.88-1.78 (m, 2H), 1.77-1.64 (m, 2H).

### Example 82. 4-((1R,5S)-8-(Bis(4-fluorophenyl)methyl)-3,8-diazabicyclo[3.2.1]octan-3-yl)-2-methyl-1-(((S)-tetrahydrofuran-2-yl)methyl)-1H-[1,2,4]triazolo[3,4-b]purine

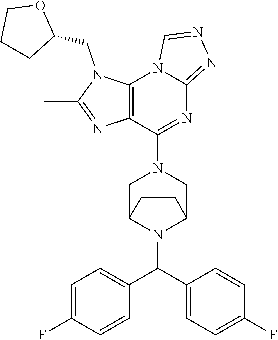 [SMILES: Cc1nc2c(N3CC4CCC(C3)N4C(c3ccc(F)cc3)c3ccc(F)cc3)nc3nncn3c2n1C[C@@H]1CCCO1]

The title compound was prepared according to the procedures described in Example 1, with (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine and (1R,5S)-8-(bis(4-fluorophenyl)methyl)-3,8-diazabicyclo[3.2.1]octane hydrochloride (Intermediate 66) replacing (2R,5S)-1-((3,3-difluorocyclobutyl)(4-(trifluoromethyl)phenyl)methyl)-2,5-dimethylpiperazine hydrochloride in Step 1. LC-MS calculated for C₃₁H₃₃F₂N₈O (M+H)⁺: m/z=571.3. found 571.3.

### Examples 83 and 84. 4-((2S,5R)-2,5-Dimethyl-4-((S)-2-methyl-1-(6-(trifluoromethyl)quinolin-2-yl)propyl)piperazin-1-yl)-1-(((S)-tetrahydrofuran-2-yl)methyl)-1H-[1,2,4]triazolo[3,4-b]purine and 4-((2S,5R)-2,5-dimethyl-4-((R)-2-methyl-1-(6-(trifluoromethyl)quinolin-2-yl)propyl)piperazin-1-yl)-1-(((S)-tetrahydrofuran-2-yl)methyl)-1H-[1,2,4]triazolo[3,4-b]purine

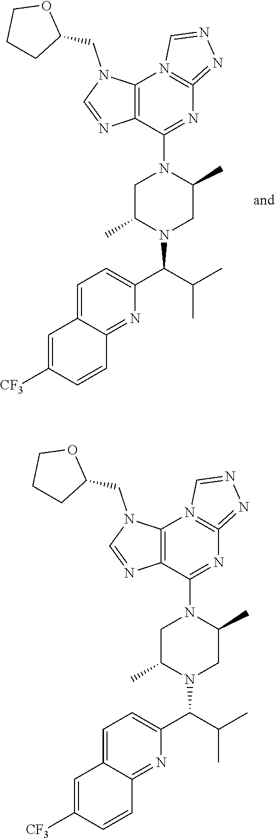

The title compounds were prepare according to the procedures described in Example 6, with 2-(1-((2R,5S)-2,5-dimethylpiperazin-1-yl)-2-methylpropyl)-6-(trifluoromethyl)quinoline dihydrochloride (Intermediate 61) replacing (2R,5S)-1-((4-chlorophenyl)((S)-2,2-difluorocyclopropyl)methyl)-2,5-dimethylpiperazine hydrochloride (Intermediate 16) in Step 1.

Example 83: Retention time on LC-MS tᵣ=1.13 min. LC-MS calculated for C₃₁H₃₇F₃N₉O (M+H)⁺: m/z=608.3. found 608.4.

Example 84: Retention time on LC-MS tᵣ=1.21 min. LC-MS calculated for C₃₁H₃₇F₃N₉O (M+H)⁺: m/z=608.3. found 608.4.

### Examples 85 and 86: 4-((2S,5R)-4-((S)-1-(4-Chlorophenyl)propyl)-2,5-dimethylpiperazin-1-yl)-2-methyl-1-(((S)-tetrahydrofuran-2-yl)methyl)-1H-[1,2,4]triazolo[3,4-b]purine and 4-((2S,5R)-4-((R)-1-(4-chlorophenyl)propyl)-2,5-dimethylpiperazin-1-yl)-2-methyl-1-(((S)-tetrahydrofuran-2-yl)methyl)-1H-[1,2,4]triazolo[3,4-b]purine

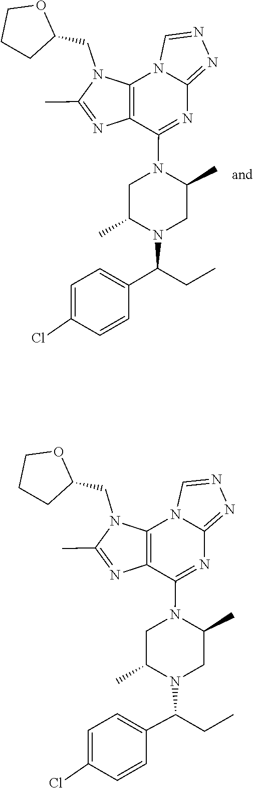

The title compounds were prepared according to the procedures outlined in Steps 2-4 for Example 7, with (2R,5S)-1-(1-(4-chlorophenyl)propyl)-2,5-dimethylpiperazine hydrochloride (Intermediate 55) replacing (2R,5S)-1-((4-chlorophenyl)((R)-2,2-difluorocyclopropyl)methyl)-2,5-dimethylpiperazine hydrochloride and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The crude reaction mixture was purified by prep-HPLC (Sunfire C18 column, eluting with a gradient of acetonitrile/water containing 0.1% TFA, at flow rate of 60 mL/min) to afford a diastereomeric mixture of the title compounds as TFA salts. The diastereomeric mixture was dissolved in CH₂Cl₂ (2 mL) and 1 M NaOH (5 mL) was added. The layers were separated and the aqueous layer was extracted with CH₂Cl₂ (5×2 mL). The combined organic layers were dried over MgSO₄ and concentrated in vacuo. The residue was purified by normal phase preparative chiral HPLC (Phenomenex LUX Amylose-2 column, 250×21.2 mm, 5 μm, eluting with a gradient of 25% EtOH in hexanes, at a flow rate of 20 mL/min) to afford the title compounds as separated diastereomers.

Example 85: Retention time on Phenomenex LUX Amylose-2 column column tᵣ=28 min., LC-MS calculated for C₂₇H₃₆ClN₈O (M+H)⁺: m/z=523.3. found 523.3.

Example 86: Retention time on Phenomenex LUX Amylose-2 column column tᵣ=32 min., LC-MS calculated for C₂₇H₃₆ClN₈O (M+H)⁺: m/z=523.3. found 523.3.

### Example 88. 4-((2S,5R)-4-(1-(4-Chloro-3-fluorophenyl)ethyl)-2,5-dimethylpiperazin-1-yl)-1-(((S)-tetrahydrofuran-2-yl)methyl)-1H-[1,2,4]triazolo[3,4-b]purine

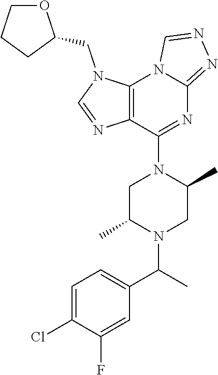 [SMILES: CC(c1ccc(Cl)c(F)c1)N1C[C@H](C)N(c2nc3nncn3c3c2ncn3C[C@@H]2CCCO2)C[C@H]1C]

The title compound was prepared according to the procedures described in Examples 41 and 42, with 1-(4-chloro-3-fluorophenyl)ethan-1-ol replacing 1-(4-(trifluoromethyl)phenyl)ethan-1-ol in Step 1, tert-butyl (2S,5R)-2,5-dimethylpiperazine-1-carboxylate replacing tert-butyl (2S,5R)-5-ethyl-2-methylpiperazine-1-carboxylate in Step 2, and (S)-2,6-dichloro-9-((tetrahydrofuran-2-yl)methyl)-9H-purine (Intermediate 1) replacing (S)-2,6-dichloro-8-methyl-9-((tetrahydrofuran-2-yl)methyl)-9H-purine (Intermediate 5) in Step 4. The title compound was purified by prep-HPLC (Sunfire C18 column, eluting with a gradient of acetonitrile/water containing 0.1% TFA, at flow rate of 60 mL/min) to afford a mixture of diastereomers as TFA salts. LC-MS calculated for C₂₅H₃₁ClFN₈O (M+H)⁺: m/z=513.2. found 513.2.

### Examples 89 and 90: 4-((2S,5R)-2,5-Dimethyl-4-((S)-1-(4-(trifluoromethyl)phenyl)propyl)piperazin-1-yl)-2-methyl-1-(((S)-tetrahydrofuran-2-yl)methyl)-1H-[1,2,4]triazolo[3,4-b]purine and 4-((2S,5R)-2,5-dimethyl-4-((R)-1-(4-(trifluoromethyl)phenyl)propyl)piperazin-1-yl)-2-methyl-1-(((S)-tetrahydrofuran-2-yl)methyl)-1H-[1,2,4]triazolo[3,4-b]purine

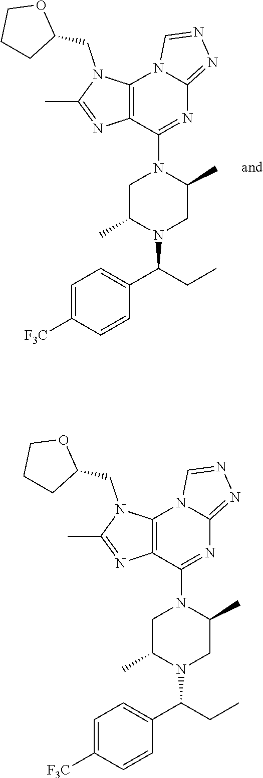 [SMILES: CC[C@@H](c1ccc(C(F)(F)F)cc1)N1C[C@H](C)N(c2nc3nncn3c3c2nc(C)n3C[C@@H]2CCCO2)C[C@H]1C ; CC[C@H](c1ccc(C(F)(F)F)cc1)N1C[C@H](C)N(c2nc3nncn3c3c2nc(C)n3C[C@@H]2CCCO2)C[C@H]1C]

The title compounds were prepared according to the procedures outlined in Steps 2-4 for Example 7, with (2R,5S)-1-(1-(4-(trifluoromethyl)phenyl)propyl)-2,5-dimethylpiperazine hydrochloride (Intermediate 57) replacing (2R,5S)-1-((4-chlorophenyl)((R)-2,2-difluorocyclopropyl)methyl)-2,5-dimethylpiperazine hydrochloride and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The crude reaction mixture was purified by prep-HPLC (Sunfire C18 column, eluting with a gradient of acetonitrile/water containing 0.1% TFA, at flow rate of 60 mL/min) to afford a diastereomeric mixture of the title compounds as TFA salts. The diastereomeric mixture was further purified by normal phase preparative chiral HPLC (Phenomenex LUX Amylose-2 column, 250×21.2 mm, 5 μm, eluting with a gradient of 20% EtOH in hexanes, at a flow rate of 20 mL/min) to afford the title compounds as separated diastereomers.

Example 89: Retention time on Phenomenex LUX Amylose-2 column tᵣ=34 min., LC-MS calculated for C₂₈H₃₆F₃N₈O (M+H)⁺: m/z=557.3. found 557.3.

Example 90: Retention time on Phenomenex LUX Amylose-2 column tᵣ=37 min., LC-MS calculated for C₂₈H₃₆F₃N₈O (M+H)⁺: m/z=557.3. found 557.3.

### Example A. In Vitro DGKα and DGKζ Inhibition Assays

The DGKα and DGKζ biochemical reactions were performed using His-tagged human recombinant enzymes (Signal Chem, DGKα, #D21-10BH; DGKζ, #D30-10H)) and DLG (Dilauroyl-sn-glycerol) lipid substrate (Signal Chem, #D430-59). ADP-Glo assay was performed using ADP-Glo™ kinase Assay kit (Promega, #V9104). The reactions were carried out in assay buffer containing 40 mM Tris, pH 7.5, 0.1% CHAPS, 0.1% Prionex, 40 mM NaCl, 5 mM MgCl₂, 1 mM CaCl₂, and 1 mM DTT. DGKα reactions contained 0.1 nM DGKα, 50 μM ATP, and 20 μM DLG. And DGKζ reactions contained 0.4 nM DGKζ, 30 μM ATP, and 20 μM DLG.

For compound inhibition studies, 40 nL test compound in DMSO was added to wells of white polystyrene plates in 384-well (Greiner, #784075) or 1536-well format (Greiner, #782075). Compounds were added with top concentration of 2 mM with 11 point, 3-fold dilution series. Enzyme solution (contains 2×DGK enzyme concentration in 1× assay buffer) was added to the plate in 2 μL/well volume, followed by 2 μL/well of substrate solution (contains 2× concentration of ATP and DLG substrate in 1× assay buffer). Plates were then centrifuged for 1 min at 1200 RPM and sealed or lidded. For 4 μL reaction volume, test compounds were therefore diluted 100× to final top concentration of 20 μM. After 90 minute incubation, reactions were quenched by addition of 2 μL/well Promega ADP-Glo Reagent, followed by centrifugation and lidding. After 60 min incubation, 2 μL/well Promega Kinase Detection Reagent was added, plates centrifuged, and incubated for 30 min. Plates were then read using Luminescence method on BMG PHERAstar FSX plate reader. Percent inhibition was calculated and IC50s were determined using 4-parameter fit in Genedata Screener. Labcyte Echo acoustic dispenser was used for compound addition, and Formulatrix Tempest liquid handler was used for all reagent dispenses.

TABLE A

| Example | DGKα IC50 (nM) | DGKζ IC50 (nM) |
|---|---|---|
| 1 | + | + |
| 2 | + | + |
| 3 | + | + |
| 4 | + | + |
| 5 | + | + |
| 6 | + | + |
| 7 | + | + |
| 8 | + | + |
| 9 | + | + |
| 10 | + | + |
| 11 | + | + |
| 12 | + | + |
| 13 | + | + |
| 14 | + | + |
| 15 | + | + |
| 16 | + | + |
| 17 | + | + |
| 18 | + | + |
| 19 | + | + |
| 20 | + | + |
| 21 | + | + |
| 22 | + | + |
| 23 | + | + |
| 24 | + | + |
| 25 | + | + |
| 26 | + | + |
| 27 | + | + |
| 28 | + | + |
| 29 | + | + |
| 30 | + | + |
| 31 | + | + |
| 32 | + | + |
| 33 | + | + |
| 34 | + | + |
| 35 | + | + |
| 36 | + | + |
| 37 | + | + |
| 38 | + | + |
| 39 | + | + |
| 40 | + | + |
| 41 | + | + |
| 42 | + | + |
| 43 | + | + |
| 44 | + | + |
| 45 | + | + |
| 46 | + | + |
| 47 | + | + |
| 48 | + | + |
| 49 | + | + |
| 50 | + | ++ |
| 51 | + | + |
| 52 | + | + |
| 53 | + | + |
| 54 | + | + |
| 55 | + | + |
| 56 | + | + |
| 57 | + | ++ |
| 58 | + | + |
| 59 | + | + |
| 60 | + | + |
| 61 | + | + |
| 62 | + | + |
| 63 | + | + |
| 64 | + | + |
| 65 | + | + |
| 66 | + | + |
| 67 | + | + |
| 68 | + | + |
| 69 | + | + |
| 70 | + | + |
| 71 | + | + |
| 72 | + | + |
| 73 | + | + |
| 74 | + | + |
| 75 | + | + |
| 76 | + | + |
| 77 | + | + |
| 78 | + | + |
| 79 | + | + |
| 80 | + | + |
| 81 | + | + |
| 82 | + | + |
| 83 | ++ | + |
| 84 | + | + |
| 85 | + | + |
| 86 | + | + |
| 88 | ++ | + |
| 89 | + | + |
| 90 | + | + |

+ refers to IC₅₀ of ≤20 nM
++ refers to IC₅₀ of >20 nM to ≤200 nM
+++ refers to IC₅₀ of >200 nM to ≤2000 nM
++++ refers to >2000 nM

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound, which is selected from:
4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(difluoromethyl)-3-fluorophenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(difluoromethyl)-2-fluorophenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(2-fluoro-4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-2-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(cyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3R)-1-(4-chlorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3S)-1-(4-chlorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(((S)-2,2-difluorocyclopropyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-(3-methyl-1-(4-(trifluoromethyl)phenyl)butyl)piperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3R)-1-(4-chloro-3-fluorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3S)-1-(4-chloro-3-fluorophenyl)-4,4,4-trifluoro-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(5-(trifluoromethyl)pyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-(bis(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((S)-(4-chlorophenyl)(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-(4-chlorophenyl)(5-chloropyridin-2-yl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(3-fluoro-4-methoxyphenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-(difluoromethyl)-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-1-(4-bromo-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-1-(4-bromo-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-1-(4-chloro-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-1-(4-chloro-2-fluorophenyl)-2-methylpropyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chloro-2-fluorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-bromophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-5-ethyl-2-methyl-4-(4-(trifluoromethyl)benzyl)piperazin-1-yl)-2-methyl-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-(4-(trifluoromethyl)benzyl)piperazin-1-yl)-2-methyl-1-(((S)-tetrahydrofuran-2-yl)methyl)-1H-[1,2,4]triazolo[3,4-b]purine;
4-((2S,5R)-2,5-dimethyl-4-(4-(trifluoromethyl)benzyl)piperazin-1-yl)-1-(((S)-tetrahydrofuran-2-yl)methyl)-1H-[1,2,4]triazolo[3,4-b]purine;
4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-4-(bis(4-fluorophenyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
1-((4-((2S,5R)-4-(bis(4-chlorophenyl)methyl)-2,5-dimethylpiperazin-1-yl)-1H-[1,2,4]triazolo[3,4-b]purin-1-yl)methyl)cyclopentan-1-ol;
1-((4-((2S,5R)-4-((4-chlorophenyl)(3,3-difluorocyclobutyl)methyl)-2,5-dimethylpiperazin-1-yl)-1H-[1,2,4]triazolo[3,4-b]purin-1-yl)methyl)cyclopentan-1-ol;
1-((4-((2S,5R)-4-((3,3-difluorocyclobutyl)(4-(trifluoromethyl)phenyl)methyl)-2,5-dimethylpiperazin-1-yl)-1H-[1,2,4]triazolo[3,4-b]purin-1-yl)methyl)cyclopentan-1-ol;
4-((2S,5R)-4-(4-chlorobenzyl)-2,5-dimethylpiperazin-1-yl)-1-(((S)-tetrahydrofuran-2-yl)methyl)-1H-[1,2,4]triazolo[3,4-b]purine;
4-((2S,5R)-4-(4-chloro-2-fluorobenzyl)-2,5-dimethylpiperazin-1-yl)-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-(1-(4-chlorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)(cyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-(4-chlorophenyl)((R)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-(4-chlorophenyl)((R)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-((S)-1-(4-(trifluoromethyl)phenyl)propyl)piperazin-1-yl)-1-(((S)-tetrahydrofuran-2-yl)methyl)-1H-[1,2,4]triazolo[3,4-b]purine;
4-((2S,5R)-2,5-dimethyl-4-((R)-1-(4-(trifluoromethyl)phenyl)propyl)piperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-2,5-dimethyl-4-((S)-2-methyl-1-(6-(trifluoromethyl)quinolin-2-yl)propyl)piperazin-1-yl)-2-methyl-1-(((S)-tetrahydrofuran-2-yl)methyl)-1H-[1,2,4]triazolo[3,4-b]purine;
4-((2S,5R)-2,5-dimethyl-4-((R)-2-methyl-1-(6-(trifluoromethyl)quinolin-2-yl)propyl)piperazin-1-yl)-2-methyl-1-(((S)-tetrahydrofuran-2-yl)methyl)-1H-[1,2,4]triazolo[3,4-b]purine;
4-((2S,5R)-4-((S)-1-(4-chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((R)-1-(4-chlorophenyl)-2-methylpropyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-2,5-dimethyl-4-(1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-2,5-dimethyl-4-(1-(4-(trifluoromethyl)phenyl)ethyl)piperazin-1-yl)-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-(4-chlorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-2-fluorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(3,4-dichlorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-2,6-difluorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-3-fluoro-5-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(3-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-3-fluorobenzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(4-chloro-3-fluorobenzyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-2,5-difluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-2,3-difluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((1R,5S)-8-((3,3-difluorocyclobutyl)(4-(trifluoromethyl)phenyl)methyl)-3, 8-diazabicyclo[3.2.1]octan-3-yl)-2-methyl-1-(((S)-tetrahydrofuran-2-yl)methyl)-1H-[1,2,4]triazolo[3,4-b]purine;
4-((1R,5S)-8-(bis(4-fluorophenyl)methyl)-3,8-diazabicyclo[3.2.1]octan-3-yl)-2-methyl-1-(((S)-tetrahydrofuran-2-yl)methyl)-1H-[1,2,4]triazolo[3,4-b]purine;
4-((2S,5R)-2,5-dimethyl-4-((S)-2-methyl-1-(6-(trifluoromethyl)quinolin-2-yl)propyl)piperazin-1-yl)-1-(((S)-tetrahydrofuran-2-yl)methyl)-1H-[1,2,4]triazolo[3,4-b]purine;
4-((2S,5R)-2,5-dimethyl-4-((R)-2-methyl-1-(6-(trifluoromethyl)quinolin-2-yl)propyl)piperazin-1-yl)-1-(((S)-tetrahydrofuran-2-yl)methyl)-1H-[1,2,4]triazolo[3,4-b]purine;
4-((2S,5R)-4-((S)-1-(4-chlorophenyl)propyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chloro-3-fluorophenyl)ethyl)-2,5-dimethylpiperazin-1-yl)-1-(((S)-tetrahydrofuran-2-yl)methyl)-1H-[1,2,4]triazolo[3,4-b]purine;
4-((2S,5R)-2,5-dimethyl-4-((S)-1-(4-(trifluoromethyl)phenyl)propyl)piperazin-1-yl)-2-methyl-1-(((S)-tetrahydrofuran-2-yl)methyl)-1H-[1,2,4]triazolo[3,4-b]purine; and
4-((2S,5R)-2,5-dimethyl-4-((R)-1-(4-(trifluoromethyl)phenyl)propyl)piperazin-1-yl)-2-methyl-1-(((S)-tetrahydrofuran-2-yl)methyl)-1H-[1,2,4]triazolo[3,4-b]purine
or a pharmaceutically acceptable salt thereof.

2. The compound of claim 1, or a pharmaceutically acceptable salt thereof, wherein the compound is deuterated.

3. A pharmaceutical composition, comprising a compound of claim 1, or a pharmaceutically acceptable salt thereof, and a pharmaceutically acceptable carrier.

4. A method of inhibiting an activity of a diacylglycerol kinase, comprising contacting the kinase with a compound of claim 1, or a pharmaceutically acceptable salt thereof.

5. A method of treating cancer in a patient in need thereof, the method comprising administering to the patient a therapeutically effective amount of a compound of claim 1, or a pharmaceutically acceptable salt thereof.

6. The method of claim 5, wherein the cancer is non-small cell lung cancer, bladder urothelial carcinoma, esophageal carcinoma, stomach adenocarcinoma, mesothelioma, liver hepatocellular carcinoma, diffuse large B cell lymphoma, kidney renal clear cell carcinoma, head and neck squamous cell carcinoma, cholangiocarcinoma, cervical squamous cell carcinoma, endocervical adenocarcinoma, and melanoma.

7. The method of claim 6, wherein the melanoma is metastatic melanoma.

8. The method of claim 1, wherein the cancer is a tumor.

9. The method of claim 8, wherein the tumor comprises high microsatellite instability (MSI-H), mismatch repair deficient (MMRd), high tumor mutational burden (TMB-H), or mismatch repair deficient (MMRd) and high tumor mutational burden (TMB-H).
